(19) United States
(12) Patent Application Publication
(10) Pub. No.: US 2023/0037234 A1
(43) Pub. Date: Feb. 2, 2023

(54) ENGINEERED CELLS FOR PRODUCTION OF CANNABINOIDS AND OTHER MALONYL-CoA-DERIVED PRODUCTS

(71) Applicant: Genomatica, Inc., San Diego, CA (US)

(72) Inventors: Jingyi Li, San Diego, CA (US); Pierre DeWals, San Diego, CA (US); Andreas Schirmer, San Diego, CA (US); Sankha Ghatak, San Diego, CA (US); David Ryan Georgianna, San Diego, CA (US)

(21) Appl. No.: 17/780,421

(22) PCT Filed: Nov. 25, 2020
(86) PCT No.: PCT/US2020/062308

§ 371 Date: May 26, 2022

Related U.S. Application Data
(60) Provisional application No. 62/941,551, filed on Nov. 27, 2019.
(60) Provisional application No. 63/044,736, filed on Jun. 26, 2020.

(51) Int. Cl. C12N 15/52; C12P 7/42; C12P 19/32; C12P 7/06

(52) U.S. Cl. CPC C12N 15/52; C12P 7/42; C12P 19/32; C12P 7/06; C12Y 203/01009; C12Y 404/01026

(57) ABSTRACT

The invention relates to engineered microorganisms (e.g., E. coli) and associated improvements for increasing the production cannabinoids (e.g. CBGA) or precursors or derivatives thereof.

### CROSS REFERENCE TO RELATED APPLICATIONS

This application claims the benefit of U.S. Provisional Patent Application Ser. No. 62/941,551 filed Nov. 27, 2019, and U.S. Provisional Patent Application Ser. No. 63/044,736 filed Jun. 26, 2020, both entitled ENGINEERED CELLS FOR PRODUCTION OF CANNABINOIDS AND OTHER MALONYL-CoA-DERIVED PRODUCTS, the disclosures of which are incorporated herein by reference. The entire content of the ASCII text file entitled “GNO0118WO_Sequence_Listing.txt” created on Nov. 25, 2020, having a size of 2323 kilobytes is incorporated herein by reference.

### FIELD OF THE INVENTION

The invention relates to engineered microorganisms and associated improvements for increasing the availability of malonyl-CoA inside the cell and for the production of cannabinoids and other malonyl-CoA-derived products or derivatives thereof.

### BACKGROUND OF THE INVENTION

The following description of the background of the invention is provided simply as an aid in understanding the invention and is not admitted to describe or constitute prior art to the invention.

Cannabinoids are prenylated isoprenoids found naturally in the plant Cannabis sativa. A Cannabis sativa plant may contain over a hundred different cannabinoids which may have different physiological effects. For example, the cannabinoid tetrahydrocannabinol (THC) is responsible for the well-known psychotropic effects of Cannabis extracts, whereas cannabidiol (CBD) lacks these effects but has been demonstrated to reduce inflammation. Recently, CBD and other cannabinoids have drawn a wider and significant scientific interest their potential to treat a wide array of disorders including insomnia, chronic pain, epilepsy, and post-traumatic stress disorder (Babson et al. (2017) Curr. Psychiatry Rep. 19:23; Romero-Sandoval et al. (2017) Curr. Rheumatol. Rep. 19:67; O'Connell et al. (2017) Epilepsy Behav. 70:341-348; Zir-Aviv et al. (2016) Behav. Pharmacol. 27:561-569). Further research into this class of compounds depends upon achieving higher levels of purity and producing greater quantities than ever before. However, purifying individual cannabinoid species from the Cannabis sativa plant is time-consuming and costly, and often results in low yields of many minor cannabinoid species which may be present as only a small fraction of the total cannabinoid in the plant. Thus, engineered cells are a useful alternative for the production of a specific cannabinoid or cannabinoid precursor, and may be used to increase the quantity, purity, and efficiency of cannabinoid production.

The cannabinoid pathway is complex, requiring the synthesis of several precursors necessary for the production of cannabigerolic acid (CBGA), the “mother cannabinoid”, or analogs thereof, from which other cannabinoids may be synthesized. Many of the CBGA precursors and other reactants in the cannabinoid synthetic pathways are limiting and/or are used in competing pathways in naturally-occurring and engineered host cells. Thus, it is desirable to develop engineered cells (e.g., microorganisms) that are modified for increased production of cannabinoid precursors and reactants, CBGA, and/or other cannabinoids or derivatives thereof.

### SUMMARY

In one aspect, the invention provides an engineered cell for producing a cannabinoid (or derivative thereof), wherein the cell comprises one or more of the following modifications:

(i) express an exogenous nucleic acid sequence encoding an olivetol synthase;
(ii) express an exogenous nucleic acid sequence encoding an olivetolic acid cyclase;
(iii) express an exogenous nucleic acid sequence encoding a prenyltransferase; and one or more of the following modifications:
(iv) express one or more exogenous nucleic acid sequences or overexpress one or more endogenous genes encoding a protein having an ABC transporter permease;
(v) express one or more exogenous nucleic acid sequences or overexpress one or more endogenous genes encoding a protein having an ABC transporter ATP-binding protein activity;
(vi) express one or more exogenous nucleic acids sequences or overexpress one or more endogenous genes that encodes a protein that is at least 60% identical to: the blc gene product of SEQ ID NO: 147, ybhG gene product of SEQ ID NO: 116, or the ydhC gene product of SEQ ID NO: 148, or EmrB/QacA subfamily drug resistance transporters, such as the pur8 protein, of one of SEQ ID NOs: 210-214;
(vii) express one or more exogenous nucleic acids sequences or overexpress one or more endogenous genes that encodes a protein that is at least 60% identical to the mlaD gene product of SEQ ID NO: 149, the mlaE gene product of SEQ ID NO: 150, or the mlaF gene product of SEQ ID NO: 151;
(viii) express one or more exogenous nucleic acid sequences or overexpress one or more endogenous genes encoding a protein having a siderophore receptor protein activity or overexpress one or more endogenous genes encoding a protein having a siderophore receptor protein activity;
(ix) a disruption of or downregulation in the expression of a regulator of expression of one or more endogenous genes encoding a protein having an ABC transporter permease activity, a protein having an ABC transporter ATP-binding protein activity, a blc gene, a ybhG protein, a ydhC protein, an EmrB/QacA subfamily drug resistance transporter, a mlaD protein, mlaE protein, mlaF protein, or a protein having a siderophore receptor protein activity;
(x) express an exogenous nucleic acid encoding a multi-domain protein having acetyl-CoA carboxylase activity (MD-ACC);
(xi) overexpress one or more endogenous genes encoding acetyl-CoA carboxyltransferase subunit α, biotin carboxyl carrier protein, biotin carboxylase, or acetyl-CoA carboxyltransferase subunit β, or express one or more exogenous genes encoding acetyl-CoA carboxyltransferase, biotin carboxyl carrier protein, or biotin carboxylase activities;
(xii) disruption of or downregulation in the expression of an endogenous gene encoding a protein having (acyl-carrier-protein)S-malonyltransferase activity, an endogenous gene encoding a protein having 3-hydroxypalmitoyl-(acyl-carrier-protein) dehydratase activity, or both;
(xiii) express an exogenous nucleic acid sequence or overexpress an endogenous gene encoding a protein having fatty acyl-CoA ligase activity, or both;
(xiv) disruption of or downregulation in the expression of at least one endogenous gene encoding a protein having acyl-CoA dehydrogenase activity or enoyl-CoA hydratase activity;
(xv) a disruption or downregulation in the expression of at least one endogenous gene encoding a protein having acyl-CoA esterase/thioesterase activity;
(xvi) disruption of or downregulation in the expression of at least one endogenous gene encoding a repressor of transcription of one or more genes required for fatty acid beta-oxidation or an upregulator of fatty acid biosynthesis in combination with disruption or downregulation of one or more endogenous genes encoding one or more proteins of fatty acid beta-oxidation pathway;
(xvii) express an exogenous nucleic acid sequence or overexpress an endogenous gene encoding a protein having prenol kinase activity, prenol diphosphokinase activity, isoprenol kinase activity, isoprenol diphosphokinase activity, dimethylallyl phosphate kinase activity, isopentenyl (di)phosphate kinase activity, or isopentenyl diphosphate isomerase activity;
(xviii) express an exogenous nucleic acid sequence or overexpress an endogenous gene encoding a protein having GPP synthase activity;
(xix) express one or more exogenous nucleic acid sequences or overexpressing one or more endogenous genes encoding one or more enzymes of MVA pathway, MEP pathway, or a non-MVA, non-MEP pathway;
(xx) express an exogenous nucleic acid sequence or overexpress an endogenous gene encoding a biotin-(acetyl-CoA carboxylase) ligase;
(xxi) overexpress an endogenous gene encoding a isopentenyl-diphosphate delta-isomerase or express an exogenous nucleic acid sequence encoding a isopentenyl-diphosphate delta-isomerase;
(xxii) overexpress an endogenous genes encoding a hydroxyethylthiazole kinase or express an exogenous nucleic acid sequence encoding a hydroxyethylthiazole kinase or both;
(xxiii) express an exogenous nucleic acid sequence encoding a Type III pantothenate kinase or overexpress an endogenous gene encoding a Type III pantothenate kinase;
(xxiv) a disruption of or downregulation in the expression of at least one endogenous gene encoding a phosphatase selected from the group consisting of ADP-sugar pyrophosphatase, dihydroneopterin triphosphate diphosphatase, pyrimidine deoxynucleotide diphosphatase, pyrimidine pyrophosphate phosphatase, and Nudix hydrolase; wherein the engineered cell produces a cannabinoid (or derivative thereof);
(xxv) express one or more exogenous nucleic acid sequences or overexpress one or more endogenous genes encoding a protein that is a resistance-nodulation/-cell division (RND) transporter:
(xxvi) express one or more exogenous nucleic acid sequences or overexpress one or more endogenous genes encoding a protein that is a prokaryotic small multidrug (SMR) transporter; and
(xxvii) express one or more exogenous nucleic acid sequences or overexpress one or more endogenous genes encoding a protein that is a member of the major facilitator superfamily (MFS). In some embodiments, the engineered cell comprises at least two, three, four, five, six, seven, eight, nine, ten or more of the modifications. In some embodiments, the engineered cell comprises at least one, two, three, four, five, six, seven, eight, or more heterologous genes. In some embodiments, the engineered cell comprises and/or expresses at least one, two, three, four, five, six, seven, eight, or more non-naturally occurring proteins. In some embodiments, the engineered cell comprises the deletion or disruption of one, two, three, four, five, six, seven, eight or more endogenous genes. In some embodiments, the cell is engineered to express one, two, three, four, five, six, seven, eight or more endogenous genes, wherein the native promoter(s) of the endogenous gene(s) is/are replaced with one or more promoters that increase expression of the gene(s) relative to the expression in a control cell under the control of the native promoter. In some embodiments, the cell is engineered to express one, two, three, four, five, six, seven, eight, or more exogenous genes or overexpress one, two, three, four, five, six, seven, eight, or more endogenous genes in which one or more exogenous or endogenous gene is a non-natural variant of the naturally occurring endogenous gene. In some embodiments, the non-natural variant of the exogenous or endogenous gene comprise one or more amino acid substitutions, insertions, or deletions as compared to the naturally occurring genes.

In some embodiments the engineered cell expresses (a) exogenous nucleic acid sequences encoding (a1) olivetol synthase, (a2) olivetolic acid cyclase, (a3) prenyltransferase, and (a4) one or more genes of a MVA pathway, MEP pathway, or a non-MVA, non-MEP pathway; and (b) one or more of the following: (b1) an exogenous nucleic acid encoding a multi-domain protein having acetyl-CoA carboxylase activity (MD-ACC), or overexpress one or more endogenous genes encoding acetyl-CoA carboxyltransferase subunit α, biotin carboxyl carrier protein, biotin carboxylase, or acetyl-CoA carboxyltransferase subunit β, or expresses one or more exogenous genes encoding acetyl-CoA carboxyltransferase, biotin carboxyl carrier protein, or biotin carboxylase activities, (b2) express an exogenous nucleic acid sequence or overexpress an endogenous gene encoding a protein having fatty acyl-CoA ligase activity, or both; disruption of or downregulation in the expression of at least one endogenous gene encoding a repressor of transcription of one or more genes required for fatty acid beta-oxidation or an upregulator of fatty acid biosynthesis in combination with disruption or downregulation of one or more endogenous genes encoding one or more proteins of fatty acid beta-oxidation pathway; a disruption or downregulation in the expression of at least one endogenous gene encoding a protein having 3-oxoacyl-[acyl-carrier-protein] synthase activity, an endogenous gene encoding a protein having enoyl-[acyl-carrier-protein] reductase activity, and (b3) expresses one or more exogenous nucleic acid sequences or overexpress one or more endogenous genes encoding a protein having an ABC transporter permease; expresses one or more exogenous nucleic acid sequences or overexpress one or more endogenous genes encoding a protein having an ABC transporter ATP-binding protein activity; expresses one or more exogenous nucleic acids sequences or overexpress one or more endogenous genes that encodes a protein that is at least 60% identical to: the blc gene product of SEQ ID NO: 147, ybhG gene product of SEQ ID NO: 116, or the ydhC gene product of SEQ ID NO: 148, or EmrB/QacA subfamily drug resistance transporters, such as the pur8 protein, of one of SEQ ID NOs: 210-214, or expresses one or more exogenous nucleic acids sequences or overexpress one or more endogenous genes that encodes a protein that is at least 60% identical to the mlaD gene product of SEQ ID NO: 149, the mlaE gene product of SEQ ID NO: 150, the mlaF gene product of SEQ ID NO: 151, or the RND family MdtABC.

In some embodiments the engineered cell expresses: (a1)-(a4) and (b1); (a1)-(a4) and (b2); (a1)-(a4) and (b3); (a1)-(a4), (b1) and (b2); (a1)-(a4), (b1) and (b3); (a1)-(a4), (b2) and (b3); (a1)-(a4) and (b1)-(b3). In some embodiments, (b1) is expression of acetyl-CoA carboxyltransferase (ACC), such as C. glutamicum or M. circinelloides ACC, or a protein having >60%, ≥65%, ≥70%, ≥75%, ≥80%, ≥85%, ≥90%, ≥95%, ≥97%, ≥99%, or 100% identity to said sequences. In some embodiments, (b2) is deletion, disruption, or reduced expression of one or more of fabA, fabB, fabD, fabF, fabG, fabH, fabL, fadE, fadD, fadI, fadM, fadL, and fadR. In some embodiments (b3) is expression of one or more of blc, ybhG, ydhC, mlaD, mlaE, mlaF, or MdtABC, or a protein having >60%, ≥65%, ≥70%, ≥75%, ≥80%, ≥85%, ≥90%, ≥95%, ≥97%, ≥99%, or 100% identity to said sequences.

In another aspect, the invention provides an engineered cell, wherein the cell is engineered to express an exogenous nucleic acid encoding a multi-domain protein having acetyl-CoA carboxylase (ACC) activity and wherein the engineered cell produces more cannabinoid or derivatives thereof or combinations thereof than is produced by a control cell substantially identical to the engineered cell with the exception that the control cell is not engineered to express the exogenous nucleic acid encoding the multi-domain protein having ACC activity.

In another aspect, the invention provides an engineered cell, wherein the cell is engineered to express:

(a) an exogenous nucleic acid encoding a multi-domain protein having acetyl-CoA carboxylase (MD-ACC) activity; and

(b) at least one of:
(i) an exogenous nucleic acid encoding a natural or non-natural olivetol synthase;
(ii) an exogenous nucleic acid encoding an olivetolic acid cyclase; and
(iii) an exogenous nucleic acid encoding a natural or non-natural prenyltransferase.

In another aspect, the invention provides an engineered cell, wherein the cell is engineered:

(a) to express an exogenous nucleic acid encoding a multi-domain protein having acetyl-CoA carboxylase (MD-ACC) activity; and
(b) to further comprise one or more of the following modifications:
(i) a disruption or downregulation in the expression of an endogenous gene encoding a protein having (acyl-carrier-protein)S-malonyltransferase activity, an endogenous gene encoding a protein having 3-hydroxypalmitoyl-(acyl-carrier-protein) dehydratase activity, or both;
(ii) express an exogenous nucleic acid sequence or overexpress an endogenous gene encoding a protein having fatty acyl-CoA ligase activity, or both;
(iii) a disruption or downregulation in the expression of at least one endogenous gene encoding a protein having acyl-CoA dehydrogenase activity or enoyl-CoA hydratase activity;
(iv) a disruption or downregulation in the expression of at least one endogenous gene encoding a protein having acyl-CoA esterase/thioesterase activity;
(v) express an exogenous nucleic acid sequence or overexpress an endogenous gene encoding a protein having prenol kinase activity, prenol diphosphokinase activity, isoprenol kinase activity, isoprenol diphosphokinase activity, dimethylallyl phosphate kinase activity, isopentenyl (di)phosphate kinase activity, or isopentenyl diphosphate isomerase activity express a heterologous nucleic acid sequence or overexpress an endogenous gene encoding a protein having GPP synthase activity, or both;
(vi) express one or more exogenous nucleic acid sequences or overexpress one or more endogenous genes encoding a protein having an ABC transporter permease activity, express one or more exogenous nucleic acid sequences or overexpress one or more endogenous genes encoding a protein having an ABC transporter ATP-binding protein activity,
(vii) express one or more exogenous genes encoding an ABC transporter permease or ABC transporter ATP-binding protein that is capable of effecting cannabinoid (or derivatives thereof) efflux from the cell, wherein the native promoter of at least one of the one or more endogenous genes is replaced with a constitutive promoter that increases expression of the genes relative to the expression in a control cell with the native promoter; and
(viii) a disruption or downregulation in the expression of an ybiH gene.

In some embodiments, one or more modifications of the engineered cell increases the availability of malonyl-CoA in the engineered cell as compared to a control cell substantially identical to the engineered cell with the exception that the control cell does not comprise one or more of such modifications. In some embodiments, one or more modifications of the engineered cell increases the production of a cannabinoid (or derivative thereof) as compared to a control cell substantially identical to the engineered cell with the exception that the control cell does not comprise one or more of such modifications. In some embodiments, one or more modifications of the engineered cell increases the efflux of cannabinoid (or derivatives thereof) from the engineered cell as compared to a control cell substantially identical to the engineered cell with the exception that the control cell does not comprise one or more of such modifications.

In some embodiments, the multi-domain protein having acetyl-CoA carboxylase activity (EC 6.4.1.2) is exogenous to an engineered cell. In some embodiments, the multi-domain protein having acetyl-CoA carboxylase activity (EC 6.4.1.2) is heterologous to an engineered cell. Optionally, the multi-domain protein is a fungal multi-domain protein having acetyl-CoA carboxylase (ACC) activity. The fungal multi-domain protein may be derived from Mucor spp, Rhizopus spp. Aspergillus spp., Saccharomyces spp., or Yarrowia spp. In some embodiments, the multi-domain protein has a sequence that is at least 60%, 65%, 70%, 75%, 80%, 85%, 90%, 95%, 97%, 99%, or 100% identical to at least 50, 100, 150, 200, 250, or more contiguous amino acids of any one of the sequences of SEQ ID NOs: 1-100 and 208-209 and has acetyl-CoA carboxylase activity (EC 6.4.1.2). In some embodiments, the multi-domain protein has a sequence that is at least 60%, 65%, 70%, 75%, 80%, 85%, 90%, 95%, 97%, 99%, or 100% identical to the full length of any one of the sequences of SEQ ID NOs: 1-100 and 208-209 and has acetyl-CoA carboxylase activity (EC 6.4.1.2).

In some embodiments, the multi-domain protein is encoded by a nucleic acid sequence that is at least 60%, 65%, 70%, 75%, 80%, 85%, 90%, 95%, 99% identical to SEQ ID NO: 101. Optionally, the multi-domain protein is encoded by a nucleic acid sequence that is identical or substantially identical to SEQ ID NO: 101

In some embodiments of any of the foregoing aspects, a cell is engineered for a modification that causes a disruption or downregulation in the expression of an endogenous gene encoding a protein having (acyl-carrier-protein)S-malonyltransferase activity, an endogenous gene encoding a protein having 3-hydroxypalmitoyl-(acyl-carrier-protein) dehydratase activity, or both.

Optionally, the protein having (acyl-carrier-protein)S-malonyltransferase activity has an enzymatic activity of EC 2.3.1.39. Optionally, the protein having the (acyl-carrier-protein)S-malonyltransferase activity has an amino acid sequence that is identical or substantially identical to SEQ ID NO: 102. The protein having the (acyl-carrier-protein)S-malonyltransferase activity may be encoded by the fabD gene.

Optionally, the protein having 3-hydroxypalmitoyl-(acyl-carrier-protein) dehydratase activity has an enzymatic activity of EC 4.2.1.59. Optionally, the protein having 3-hydroxypalmitoyl-(acyl-carrier-protein) dehydratase activity has an amino acid sequence that is identical or substantially identical to SEQ ID NO: 103. The protein having 3-hydroxypalmitoyl-(acyl-carrier-protein) dehydratase activity may be encoded by the fabZ gene.

In some embodiments, the of any of the foregoing aspects, a cell is engineered for a modification that causes a disruption or downregulation in the expression of at least one endogenous gene encoding a protein having 3-oxoacyl-[acyl-carrier-protein] synthase activity, an endogenous gene encoding a protein having enoyl-[acyl-carrier-protein] reductase activity, or both.

Optionally, the protein having 3-oxoacyl-[acyl-carrier-protein] synthase activity is identical or substantially identical to a protein selected from the group consisting of FabF (SEQ ID NO: 193), FabB (SEQ ID NO: 194), and FabH (SEQ ID NO: 195). Optionally, the protein having enoyl-[acyl-carrier-protein] reductase activity is FabI (SEQ ID NO: 196).

In some embodiments of any of the foregoing aspects, an engineered cell having a disruption or downregulation in the expression of the endogenous gene encoding a protein having (acyl-carrier-protein)S-malonyltransferase activity, the endogenous gene encoding a protein having 3-hydroxypalmitoyl-(acyl-carrier-protein) dehydratase activity, or both has more malonyl-CoA available than in a by a control cell that is substantially identical to the engineered cell without the modifications.

In some embodiments of any of the foregoing aspects, the cell is engineered to express an exogenous nucleic acid sequence or overexpress an endogenous gene encoding a protein having fatty acyl-CoA ligase activity. Optionally, the protein having fatty acyl-CoA ligase activity has an enzymatic activity of EC 6.2.1.3. The exogenous nucleic acid sequence or the endogenous gene may be a fadD gene, or a variant thereof. In some embodiments, the fadD variant may be a non-naturally occurring variant. In some embodiments, the fadD variant comprises one or more amino acid substitutions as compared to the wild-type protein. Optionally, the exogenous nucleic acid sequence or the endogenous gene encodes a protein may be identical or substantially identical to SEQ ID NO: 104. In some embodiments, the cell makes more hexanoyl-CoA available than a control cell substantially identical to the engineered cell with the exception that the control cell is not engineered to express the exogenous nucleic acid sequence or overexpress the endogenous gene encoding a protein having fatty acyl-CoA ligase activity.

In some embodiments, in any of the foregoing aspects, a cell is engineered for a modification that causes a disruption or downregulation in the expression of at least one endogenous gene encoding a protein having acyl-CoA dehydrogenase activity or enoyl-CoA hydratase activity. Optionally, the protein having acyl-CoA dehydrogenase activity has an enzymatic activity of EC 1.3.8.1. The gene encoding the protein having acyl-CoA dehydrogenase activity encodes a protein having an amino acid sequence that may be identical or substantially identical to SEQ ID NO: 105. Optionally, the gene encoding a protein having acyl-CoA dehydrogenase activity is a fadE gene.

Optionally, the protein having enoyl-CoA hydratase activity has an enzymatic activity of EC 4.2.1.17. The gene encoding a protein having enoyl-CoA hydratase activity may encode a protein having an amino acid sequence that may be identical or substantially identical to SEQ ID NO: 106.

Optionally, the gene encoding a protein having enoyl-CoA hydratase activity is a fadB gene.

Optionally, a cell is engineered for a modification that causes a disruption or downregulation in the expression of an endogenous gene encoding a protein having acyl-CoA dehydrogenase activity and an endogenous gene encoding a protein having enoyl-CoA hydratase activity. The cell may be engineered for a modification that causes a disruption or downregulation in the expression of a fadB gene and a fadE gene.

In some embodiments of any of the foregoing aspects, a cell is engineered for a modification that causes a disruption or downregulation in the expression of at least one endogenous gene encoding a protein having acyl-CoA esterase/thioesterase activity. Optionally, the protein having acyl-CoA esterase/thioesterase activity has an enzymatic activity of EC 3.1.2.20. The gene encoding a protein having acyl-CoA esterase/thioesterase activity may encode a protein having an amino acid sequence that may be identical or substantially identical to any one of SEQ ID NOs: 107-109 and 197-198. For example, the gene encoding a protein having acyl-CoA esterase/thioesterase activity may be a tesB gene, a yciA gene, a ybgC gene, a tesA gene, ydiI, or a fadM gene. The cell may make more hexanoyl-CoA available than a control cell that is substantially identical to the engineered cell with the exception that the control cell is not engineered to cause a disruption or downregulation in the expression of at least one endogenous gene encoding a protein having acyl-CoA esterase/thioesterase activity.

In some embodiments of any of the foregoing aspects, a cell is engineered to:

(a) express an exogenous nucleic acid sequence or overexpress an endogenous gene encoding a protein having isopentenyl phosphate kinase activity,

(b) express an exogenous nucleic acid sequence or overexpress an endogenous gene encoding a protein having GPP synthase activity, or

(c) both.

Optionally, the protein having isopentenyl phosphate kinase activity has an enzymatic activity of EC 2.7.4.26. The exogenous nucleic acid sequence or the endogenous gene that encodes a protein having isopentenyl phosphate kinase activity may be an IPK gene. The exogenous nucleic acid sequence or the endogenous gene encodes a protein having isopentenyl phosphate kinase activity encodes a protein may be identical or substantially identical to SEQ ID NO: 110.

Optionally, the protein having GPP synthase activity has an enzymatic activity of EC 2.5.1.-. The exogenous nucleic acid sequence or the endogenous gene that encodes a protein having GPP synthase activity may be a GPP synthase (EC 2.5.1.1), an IspA gene or an IdsA gene. The exogenous nucleic acid sequence or the endogenous gene encodes a protein having GPP synthase activity may encode a protein that is identical or substantially identical to SEQ ID NO: 111 or SEQ ID NO: 112.

Optionally, the cell makes more GPP available than a control cell substantially identical to the engineered cell with the exception that the control cell is not so engineered to:

express a heterologous nucleic acid sequence or overexpress an endogenous gene encoding a protein having isopentenyl phosphate kinase (IPK) activity,

express a heterologous nucleic acid sequence or overexpress an endogenous gene encoding a protein having GPP synthase activity, or both.

In some embodiments of any of the foregoing aspects, the cell is engineered to

(a) express one or more exogenous nucleic acid sequences or overexpress one or more endogenous genes encoding a protein having an ABC transporter permease activity,

(b) express one or more exogenous nucleic acid sequences or overexpress one or more endogenous genes encoding a protein having an ABC transporter ATP-binding protein activity,

(c) express one or more exogenous nucleic acids sequences or overexpress one or more endogenous genes that encodes a protein that is identical or substantially identical to the blc gene product (SEQ ID NO: 147) the ydhC gene product (SEQ ID NO: 148), or EmrB/QacA subfamily drug resistance transporters, such as the pur8 protein, of one of SEQ ID NOs: 210-214;

(d) express one or more exogenous nucleic acids sequences or overexpress one or more endogenous genes encodes a protein that is identical or substantially identical to the mlaD gene product (SEQ ID NO: 149), the mlaE gene product (SEQ ID NO: 150), or the mlaF gene product (SEQ ID NO: 151), or

(e) any combination of (a)-(d).

Optionally, the protein having ABC transporter permease activity has an enzymatic activity of EC 7.6.2.2. At least one of the heterologous nucleic acid sequences or the endogenous genes may be selected from the group consisting of a ybhS gene, a ybhF gene, a ybhR gene, and a ybhG gene. The ybhS gene may encode a protein that is identical or substantially identical to SEQ ID NO: 113. The ybhF gene may encode a protein that is identical or substantially identical to SEQ ID NO: 114. The ybhR gene may encode a protein that is identical or substantially identical to SEQ ID NO: 115. The ybhG gene may encode a protein that is identical or substantially identical to SEQ ID NO: 116. The protein having ABC transporter permease activity may be identical or substantially identical to the protein encoded by UniProt protein sequence Q8XYF0 (RS_RS09125; SEQ ID NO: 190) or UniProt protein sequence Q8XYE9 (RS_RS09130; SEQ ID NO: 191). Optionally, the cell effluxes more cannabinoid (or derivatives thereof) than is effluxed by a control cell substantially identical to the engineered cell with the exception that the control cell is not so engineered. In some embodiments, the cannabinoid is cannabigerolic acid (CBGA), tetrahydrocannabivarin (THCV), tetrahydrocannabivarinic acid (THCVA), cannabidivarin (CBDV), cannabidivarinic acid (CBDVA), cannabinol (CBN), cannabinolic acid (CBNA), cannabidiol (CBD), cannabidiolic acid (CBDA), cannabichromene (CBC), cannabichromenic acid (CBCA), cannabigerivarin (CBGV), cannabigerivarinic acid (CBGVA), cannabigerol (CBG), Cannabichromevarin (CBCV), Cannabichromevarinic acid (CBCVA), tetrahydrocannabinol (THC), tetrahydrocannabinolic acid (THCA), analogs, or derivatives thereof, or combinations thereof.

In some embodiments, the of any of the foregoing aspects, the cell is engineered to express one or more endogenous gene encoding an ABC transporter permease or ABC transporter ATP-binding protein that is capable of effecting cannabinoid (or derivatives thereof) efflux from the cell, wherein the native promoter of at least one of the one or more endogenous genes is replaced with a promoter that increases expression of the genes relative to a the expression in a control cell with the native promoter. Optionally, the promoter is heterologous. The one or more of the endogenous genes may be selected from a ybhS gene, a ybhF gene, a ybhR gene, and a ybhG gene.

In some embodiments of any of the foregoing aspects, the cell is engineered for a modification that causes a disruption or downregulation in the expression of an ybiH gene. Optionally, the ybiH gene encodes a protein having an amino acid sequence that is identical or substantially identical to SEQ ID NO: 117. The disruption or downregulation in the expression of the ybiH gene may cause the cell to overexpress at least one endogenous genes encoding an ABC transporter permease or ABC transporter ATP-binding protein that is capable of effecting cannabinoid (or derivatives thereof) efflux from the cell relative to the expression of the at least one endogenous genes expressed by a control cell substantially identical to the engineered cell with the exception that the control cell is not engineered to cause a disruption or downregulation in the expression of the ybiH gene. The at least one endogenous gene encoding an ABC transporter permease may be selected from the group consisting of a ybhS gene, a ybhR gene, and a ybhG gene for the endogenous gene encoding the ABC transporter ATP-binding protein is a ybhF gene. In some embodiments, the cannabinoid is CBGA, THCV, THCVA, CBDV, CBDVA, CBN, CBNA, CBD, CBDA, CBC, CBCA, CBGV, CBGVA, CBG, CBCV, CBCVA, THC, THCA, analogs, or derivatives thereof, or combinations thereof.

In some embodiments, the protein having siderophore receptor protein is identical or substantially identical to the protein encoded by UniProt protein sequence Q8XYF1 (SEQ ID NO: 192).

In some embodiments, the protein having a repressor of transcription of one or more genes required for fatty acid beta-oxidation or an upregulator of fatty acid biosynthesis has an amino acid sequence that is identical or substantially identical to SEQ ID NO: 199. In some embodiments, that protein is encoded by the fadR gene. In some embodiments, the expression of fadR is attenuated or the fadR gene is deleted. In some embodiments, the engineered cell comprising an attenuated fadR expression or deleted fadR has more alkanoyl-CoA available as compared to a control cell that is substantially identical to the engineered cell without such attenuation of fadR expression or deletion of fadR. In some embodiments, the engineered cell increases the availability of alkanoyl-CoA as compared to a control cell that is substantially identical to the engineered cell with the exception that the control cell does not comprise one or more of such modifications.

In some embodiments, the Type III pantothenate kinase has an amino acid sequence that is identical or substantially identical to SEQ ID NO: 200. In some embodiments, the gene encoding the Type III pantothenate kinase is the coaX gene. In some embodiments, the engineered cell increases the availability of alkanoyl-CoA, acetyl-CoA, or malonyl-CoA, as compared to a control cell that is substantially identical to the engineered cell with the exception that the control cell does not comprise one or more of such modifications.

In some embodiments of any of the foregoing aspects and embodiments, the cell is engineered to express an exogenous nucleic acid sequence or overexpress an endogenous gene encoding a protein having biotin ligase activity. Optionally, the protein having biotin ligase activity has an enzymatic activity of EC:6.3.4.15. In some embodiments, the gene is a BirA gene or substantially identical to a BirA gene and/or encodes a protein that is substantially identical to the protein encoded by the BirA gene.

In another aspect, the invention provides an engineered cell, wherein the cell is engineered to

(a) express one or more exogenous nucleic acid sequences or overexpress one or more endogenous genes encoding a protein having an ABC transporter permease activity,

(b) express one or more exogenous nucleic acid sequences or overexpress one or more endogenous genes encoding a protein having an ABC transporter ATP-binding protein activity,

(c) express one or more exogenous nucleic acids sequences or overexpress one or more endogenous genes that encodes a protein that is at least 90% identical to: the blc gene product (SEQ ID NO: 147), the ydhC gene product (SEQ ID NO: 148), or EmrB/QacA subfamily drug resistance transporters, such as the pur8 protein, of one of SEQ ID NOs: 210-214;

(d) express one or more exogenous nucleic acids sequences or overexpress one or more endogenous genes encodes a protein that is at least 90% identical to the mlaD gene product (SEQ ID NO: 149), the mlaE gene product (SEQ ID NO: 150), or the mlaF gene product (SEQ ID NO: 151), or

(e) any combination of (a)-(d),

wherein the engineered cell effluxes more cannabinoid (or derivative thereof) than is effluxed by a control cell.

In another aspect, the invention provides an engineered cell, wherein the cell is engineered to express:

(a) at least one of:

(i) express one or more exogenous nucleic acid sequences or overexpress one or more endogenous genes encoding a protein having an ABC transporter permease activity,
(ii) express one or more exogenous nucleic acid sequences or overexpress one or more endogenous genes encoding a protein having an ABC transporter ATP-binding protein activity,
(iii) express one or more exogenous nucleic acids sequences or overexpress one or more endogenous genes that encodes a protein that is at least 90% identical to: the blc gene product (SEQ ID NO: 147), the ydhC gene product (SEQ ID NO: 148), or EmrB/QacA subfamily drug resistance transporters, such as the pur8 protein, of one of SEQ ID NOs: 210-214;
(iv) express one or more exogenous nucleic acids sequences or overexpress one or more endogenous genes encodes a protein that is at least 90% identical to the mlaD gene product (SEQ ID NO: 149), the mlaE gene product (SEQ ID NO: 150), or the mlaF gene product (SEQ ID NO: 151), or
(v) any combination of (i)-(iv), and

(b) at least one of:

(vi) an exogenous nucleic acid encoding a natural or non-natural olivetol synthase;
(vii) an exogenous nucleic acid encoding an olivetolic acid cyclase; and
(viii) an exogenous nucleic acid encoding a natural or non-natural prenyltransferase.

In some embodiments of these aspects, the protein having ABC transporter permease activity has an enzymatic activity of EC 7.6.2.2. The heterologous nucleic acid sequences or the endogenous genes may be selected from the group consisting of a ybhS gene, a ybhF gene, a ybhR gene, and a ybhG gene. The ybhS gene may encode a protein that is identical or substantially identical to SEQ ID NO: 113. The ybhF gene may encode a protein that is identical or substantially identical to SEQ ID NO: 114. The ybhR gene may encode a protein that is identical or substantially identical to SEQ ID NO: 115. The ybhG gene may encode a protein that is identical or substantially identical to SEQ ID NO: 116. The protein having ABC transporter permease activity may be identical or substantially identical to the protein encoded by UniProt protein sequence Q8XYF0 (SEQ ID NO: 190) or UniProt protein sequence Q8XYE9 (SEQ ID NO: 191). Other ABC-type transporters include the following genes from E. coli: msbA (UniProt P60752; SEQ ID NO: 215), macAB, (UniProt P75830, P75831; SEQ ID NO: 216), mdlAB (UniProt P77265, P0AAG5; SEQ ID NO: 217), yadGH (UniProt P36879, P0AFN6; SEQ ID NO: 218), ybbAP (UniProt P0A9T8, P77504; SEQ ID NO: 219), yddA (UniProt P31826; SEQ ID NO: 220), yojI (UniProt P33941; SEQ ID NO: 221), and yhhJ (UniProt P0AGH1; SEQ ID NO: 222).

In another aspect, the invention provides an engineered cell expressing one or more endogenous gene encoding an ABC transporter permease or ABC transporter ATP-binding protein that is capable of affecting cannabinoid (or derivatives thereof) efflux from the cell, wherein the native promoter of at least one of the one or more endogenous genes is replaced with a constitutive promoter that increases expression of the genes relative to a the expression in a control cell with the native promoter. Optionally, the constitutive promoter is heterologous. One or more of the endogenous genes may be selected from a ybhS gene, a ybhF gene, a ybhR gene, a ybhG gene, a ydhC gene, a blc gene, a pur8 gene, a mlaD gene, a mlaE gene, and a mlaF gene. In some embodiments, the cannabinoid is CBGA, THCV, THCVA, CBDV, CBDVA, CBN, CBNA, CBD, CBDA, CBC, CBCA, CBGV, CBGVA, CBG, CBCV, CBCVA, THC, THCA, analogs, or derivatives thereof, or combinations thereof.

In another aspect, the invention provides an engineered cell, wherein the cell is engineered for a modification that causes a disruption or downregulation in the expression of an ybiH gene. The ybiH gene may encode a protein having an amino acid sequence that is identical or substantially identical to SEQ ID NO: 117. The disruption or downregulation in the expression of the ybiH gene may cause the cell to overexpress at least one endogenous genes encoding an ABC transporter permease or ABC transporter ATP-binding protein that is capable of effecting cannabinoid (or derivatives thereof)efflux from the cell relative to the expression of the at least one endogenous genes expressed by a control cell substantially identical to the engineered cell with the exception that the control cell is not engineered to cause a disruption or downregulation in the expression of the ybiH gene. The at least one endogenous gene encoding an ABC transporter permease may be selected from the group consisting of a ybhS gene, a ybhR gene, and a ybhG gene. The endogenous gene encoding the ABC transporter ATP-binding protein may be a ybhF gene. In some embodiments, the cell effluxes more CBGA, THCV, THCVA, CBDV, CBDVA, CBN, CBNA, CBD, CBDA, CBC, CBCA, CBGV, CBGVA, CBG, CBCV, CBCVA, THC, THCA, analogs, or derivatives thereof, or combinations thereof than is effluxed by a control cell. In some embodiments, the cell effluxes more CBGA.

In some embodiments of any of the foregoing aspects, the exogenous nucleic acid sequence is a heterologous nucleic acid sequence.

In some embodiments of any of the foregoing aspects, the engineered cell produces more cannabigerol (CBGA) than is produced by a control cell substantially identical to the engineered cell. In some embodiments, the engineered cell produces CBGA, THCV, THCVA, CBDV, CBDVA, CBN, CBNA, CBD, CBDA, CBC, CBCA, CBGV, CBGVA, CBG, CBCV, CBCVA, THC, THCA, analogs, or derivatives thereof, or combinations thereof.

In some embodiments of any of the foregoing aspects, the cell is selected from the group consisting of bacteria, fungi, yeast, cyanobacteria, and algae, including, for example, E. coli.

In another aspect, the invention provides a method for making more malonyl-CoA available as a metabolic intermediate in a microbial production pathway of the product, the method comprising

(a) combining one or more carbon sources, a cell of any one of the foregoing aspects or embodiments, and a microorganism cell culture media to produce a cell culture; and

(b) incubating the cell culture produced in step (a) under conditions that produce the product.

In some embodiments, the product is olivetolic acid or analogs thereof, a cannabinoid, and/or derivatives of a cannabinoid. In some embodiments, the cannabinoid is CBGA, THCV, THCVA, CBDV, CBDVA, CBN, CBNA, CBD, CBDA, CBC, CBCA, CBGV, CBGVA, CBG, CBCV, CBCVA, THC, THCA, analogs, or derivatives thereof, or combinations thereof.

In some embodiments, the method further comprises a step of isolating or purifying the cannabinoid from other material. The step of isolating or purifying may comprise one or more of liquid-liquid extraction, pervaporation, evaporation, filtration, membrane filtration (including reverse osmosis, nanofiltration, ultrafiltration, and microfiltration), membrane filtration with diafiltration, membrane separation, reverse osmosis, electrodialysis, distillation, extractive distillation, reactive distillation, azeotropic distillation, crystallization and recrystallization, centrifugation, extractive filtration, ion exchange chromatography, size exclusion chromatography, adsorption chromatography, carbon adsorption, hydrogenation, solvent extraction, and ultrafiltration.

In another aspect, the invention provides a cannabinoid produced by the method of any one of the foregoing aspects or embodiments.

In another aspect, the invention provides a composition comprising a cannabinoid, its analogs or derivatives and a cell culture media or component thereof, wherein the cannabinoid, its analogs or derivatives is present at a concentration of at least 5% (w/v). The CBGA may be present at a concentration of 5%-90% (w/v), including 5%-20% (w/v). In some embodiments, the cannabinoid is CBGA, THCV, THCVA, CBDV, CBDVA, CBN, CBNA, CBD, CBDA, CBC, CBCA, CBGV, CBGVA, CBG, CBCV, CBCVA, THC, THCA, analogs, or derivatives thereof, or combinations thereof.

In another aspect, the invention provides a method for making a composition comprising a cannabinoid (or derivatives thereof), the method comprising:

(a) producing a cannabinoid (or derivatives thereof) according to the method of any one of the foregoing aspects or embodiments;

(b) separating the cells from the cell culture to produce a substantially cell-free culture media; and

(c) producing a cannabinoid (or derivatives thereof) concentrate from the culture media, wherein the cannabinoid is present in a higher concentration in the cannabinoid concentrate than in the cell-free culture media.

Optionally, the cannabinoid concentrate comprises a cannabinoid concentration of at least 5%, 10%, 15%, 20%, 25%, 30%, 35%, 40%, 45%, 50%, 55%, 60%, 65%, 70%, 75%, 80%, 85%, 90% (w/v).

In another aspect, the invention provides a method for making a composition comprising cannabinoid (or derivatives thereof), the method comprising:

(a) producing cannabinoid (or derivatives thereof) according to the method of any one of the foregoing aspects or embodiments;

(b) separating the cells from the cell culture to produce a substantially cell-free culture media; and

(c) producing cannabinoid (or derivatives thereof) concentrate from the culture media, wherein the cannabinoid (or derivatives thereof) is present in a higher concentration in the cannabinoid (or derivatives thereof) concentrate than in the cell-free culture media.

Optionally, the cannabinoid (or derivatives thereof) concentrate comprises cannabinoid (or derivatives thereof) concentration of at least 10% (w/v). In some embodiments, the cannabinoid (or derivatives thereof) concentrate comprises cannabinoid (or derivatives thereof) concentration of at least 5% (w/v).

In some embodiments, the cannabinoid is present in a concentration of at least 5%, 10%, 15%, 20%, 25%, 30%, 35%, 40%, 45%, 50%, or more on a weight:volume (w/v) basis or about 5-50%, 5-40%, 5-30%, 5-20%, 10-50%, 10-40%, 10-30%, or 10-20%.

In some embodiments, the molar ratio of cannabinoid to its analogs or derivatives is about 100:0, 99.9:0.1, 99.5:0.5, 99:1, 98:2, 97.5:2.5, 97:3, 95:5, 90:10, 85:15, 80:20, 75:25, 70:30, 65:35, 60:40, 55:45, 50:50, 45:55, 40:60, 35:65, 30:70, 25:75, 20:80, 15:85, 10:90, 5:95, 2.5:97.5, 2:98, 1:99, 0.5:95, 0.1:99.9, 0.01:99.99.

In some embodiments, the composition comprising cannabinoids, analogs, or derivatives thereof is a liquid, an engineered cell fermentation broth or cell culture medium, a cell free fermentation broth, or an engineered cell lysate. In some embodiments, the engineered cell, engineered cell extract, or engineered cell culture medium comprises cannabinoid (or derivatives thereof) at a concentration of no more than about 90% to about 0.0001%, no more than about 20% to about 0.001%, no more than about 10% to about 0.01% by weight of the engineered cell, engineered cell extract, or engineered cell culture medium.

In some embodiments, the cannabinoids including cannabinoids derivatives thereof are essentially free of pesticides, heavy metals, other plant derived materials, or antibiotics. In some embodiments, the cannabinoids, and/or derivatives or analogs thereof are at least about 25%, 30%, 35%, 40%, 45%, 50%, 55%, 60%, 65%, 70%, 75%, 80%, 85%, 90%, 95%, 96%, 97%, 98%, 99%, 99.5%, 99.8% or more pure.

As used herein, the term “cannabinoid” refers to a class of compounds that include both naturally occurring and non-naturally occurring compounds, characterized and uncharacterized, thus the term “cannabinoid” as used herein encompasses cannabinoid derivatives—i.e., derivatives of naturally-occurring or known cannabinoids. Exemplary cannabinoids include cannabidiol (CBD), cannabidiolic acid (CBDA), cannabigerol (CBG), cannabigerolic acid (CBGA), cannabichromene (CBC), cannabichromenic acid (CBCA), Δ⁹-tetrahydrocannabinol 25 (THC), Δ⁹-tetrahydrocannabinoic acid (Δ⁹-THCA), cannabinol (CBN), and cannabinolic acid (CBNA). Additional cannabinoids are provided in the disclosure hereinabove. Also included are derivatives of known, naturally-occurring cannabinoids and cannabinoids disclosed herein, which can be any derivatives, and include but are not limited to derivatives having alkyl chain lengths that are longer or shorter than C6. In some embodiments, cannabinoid derivatives include non-naturally occurring cannabinoids.

Cannabinoids may include, but are not limited to, cannabichromene (CBC) type (e.g. cannabichromenic acid), cannabigerol (CBG) type (e.g. cannabigerolic acid), cannabidiol (CBD) type (e.g. cannabidiolic acid), Δ⁹-trans-tetrahydrocannabinol (Δ⁹-THC) type (e.g. Δ⁹-tetrahydrocannabinolic acid), Δ⁸-trans-tetrahydrocannabinol (Δ⁸-THC) type, cannabicyclol (CBL) type, cannabielsoin (CBE) type, cannabinol (CBN) type, cannabinodiol (CBND) type, cannabitriol (CBT) type, cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic acid (CBCA), cannabichromene (CBC), cannabichromevarinic acid (CBCVA), cannabichromevarin (CBCV), cannabidiolic acid (CBDA), cannabidiol (CBD), cannabidiol monomethylether (CBDM), cannabidiol-C4 (CBD-C4), cannabidivarinic acid (CBDVA), cannabidivarin (CBDV), cannabidiorcol (CBD-C1), Δ⁹-tetrahydrocannabinolic acid A (THCA-A), Δ⁹-tetrahydrocannabinolic acid B (THCA-B), Δ⁹-tetrahydrocannabinol (THC), Δ⁹-tetrahydrocannabinolic acid-C4 (THCA-C4), Δ⁹-tetrahydrocannabinol-C4 (THC-C4), Δ⁹-tetrahydrocannabivarinic acid (THCVA), Δ⁹-tetrahydrocannabivarin (THCV), Δ⁹-tetrahydrocannabiorcolic acid (THCA-C1), Δ⁹-tetrahydrocannabiorcol (THC-C1), iso-tetrahydrocannabivarin, Δ8-tetrahydrocannabinolic acid (Δ⁸-THCA), Δ⁸-tetrahydrocannabinol (Δ⁸-THC), cannabicyclolic acid (CBLA), cannabicyclol (CBL), cannabicyclovarin (CBLV), cannabielsoic acid A (CBEA-A), cannabielsoic acid B (CBEA-B), cannabielsoin (CBE), cannabielsoinic acid, cannabicitranic acid, cannabinolic acid (CBNA), cannabinol (CBN), cannabinol methylether (CBNM), cannabinol-C4, (CBN-C4), cannabivarin (CBV), cannabinol-C2 (CNB-C2), cannabiorcol (CBN-C1), cannabinodiol (CBND), cannabinodivarin (CBVD), cannabitriol (CBT), 10-ethyoxy-9-hydroxy-delta-6a-tetrahydrocannabinol, 8,9-dihydroxyl-delta-6a-tetrahydrocannabinol, cannabitriolvarin (CBTVE), dehydrocannabifuran (DCBF), cannabifuran (CBF), cannabichromanon (CBCN), cannabicitran (CBT), 10-oxo-delta-6a-tetrahydrocannabinol (OTHC), Δ⁹-cis-tetrahydrocannabinol (cis-THC), 3,4,5,6-tetrahydro-7-hydroxy-alpha-alpha-2-trimethyl-9-n-propyl-2,6-methano-2H-1-benzoxocin-5-methanol (OH-iso-HHCV), cannabiripsol (CBR), and trihydroxy-Δ⁹-tetrahydrocannabinol (triOH-THC).

Abbreviated terms may be used to designate cannabinoids and related molecules. For example, the term “CBGA” refers to cannabigerolic acid, “OA” refers to olivetolic acid; “CBG” refers to cannabigerol; “CBDA” refers to cannabidiolic acid; “CBD” refers to cannabidiol; “THC” refers to Δ9-tetrahydrocannabinol (Δ⁹-THC); “Δ⁸-THC” refers to Δ⁸-tetrahydrocannabinol; “THCA” refers to Δ⁹-tetrahydrocannabinolic acid (Δ⁹-THCA); “Δ⁸-THCA” refers to Δ⁸-tetrahydrocannabinolic acid; “CBCA” refers to cannabichromenic acid; “CBC” refers to cannabichromene; “CBN” refers to cannabinol; “CBDN” refers to cannabinodiol; “CBNA” refers to cannabinolic acid; “CBV” refers to cannabivarin; “CBVA” refers to cannabivarinic acid; “THCV” refers to Δ⁹-tetrahydrocannabivarin (Δ⁹-THCV); “Δ⁸-THCV” refers to Δ⁸-tetrahydrocannabivarin; “THCVA” refers to Δ⁹-tetrahydrocannabivarinic acid (Δ⁹-THCV); “Δ⁸-THCVA” refers to Δ⁸-tetrahydrocannabivarinic acid; “CBGV” refers to cannabigerovarin; “CBGVA” refers to cannabigerovarinic acid; “CBCV” refers to cannabichromevarin; “CBCVA” refers to cannabichromevarinic acid; “CBDV” refers to cannabidivarin; and “CBDVA” refers to cannabidivarinic acid.

The following description of the invention extensively refers to the synthesis of CBGA. It is understood that CBGA and its analogs and derivatives may be produced by altering the metabolic precursors in a manner that predictably alters the final product. Such alterations include, for example, altering the aliphatic chain length in fatty acid-containing precursors.

By “a protein having 3-oxoacyl-[acyl-carrier-protein] synthase activity” is meant a protein having the enzymatic activity of EC. 2.3.1.179 (e.g., encoded by FabF), EC 2.3.1.41 (e.g., encoded by FabB), and EC 2.3.1.80 (e.g., encoded by FabH).

By “protein having enoyl-[acyl-carrier-protein] reductase activity” is meant a protein having the enzymatic activity of EC 1.3.1.9 (e.g., encoded by FabI).

### BRIEF DESCRIPTION OF THE DRAWINGS

FIG. 1 is a schematic diagram illustrating the CBGA synthetic and related metabolic pathways.

FIG. 2 is a series of bar graphs showing the effect of the addition of OLA and prenol on the expression of various CBGA transporters.

FIG. 3A is a series of line graphs showing the temporal expression of mlaD, mlaE, and mlaF following the addition of OLA and prenol to the culture medium.

FIG. 3B is a series of line graphs characterizing the bacterial culture following the addition of OLA and prenol to the culture medium.

FIG. 4 shows the CGBA production of E. coli strains engineered with different geranyl diphosphate synthase genes.

FIG. 5 is a schematic diagram illustrating exemplary mevalonate pathway (MVA) and non-mevalonate pathway (MEP). The abbreviations are DXS: 1-Deoxy-D-xylulose 5-phosphate synthase; DXR: 1-Deoxy-D-xylulose 5-phosphate reductoisomerase; CMS: 2-C-methyl-D-erythritol 4-phosphate cytidylyltransferase; CMK: 4-diphosphocytidyl-2-C-methyl-D-erythritol kinase; MECS: 2-C-methyl-D-erythritol 2,4-cyclodiphosphate synthase; HDS: 4-Hydroxy-3-methyl-but-2-enyl pyrophosphate synthase; HDR: 4-Hydroxy-3-methyl-but-2-enyl pyrophosphate reductase; DMAP: Dimethylallyl pyrophosphate; AACT: acetoacetyl-CoA thiolase; HMGS: HMG-CoA synthase; HMGR: HMG-CoA reductase; MVK: mevalonate-3-kinase; PMK: Phosphomevalonate kinase; MVD: mevalonate-5-pyrophosphate decarboxylase; and IDI: isopentenyl pyrophosphate isomerase.

FIG. 6 is a schematic diagram illustrating a non-MEP, non-MVA pathway resulting in GPP synthesis from prenol.

FIG. 7 is a schematic diagram illustrating a non-MVA, non-MEP pathway resulting in GPP synthesis from isoprenol.

FIG. 8A is bar graph showing the effect of deletion of E. coli fadR gene on the production of OLA in an E. coli strain comprising OLS, OAC, prenyltransferase, thiM, fadD, Mucor circinelloides acc, deletion of E. coli fadE and fadR genes. FIG. 8B is a bar graph showing the effect of E. coli fadE gene on the production of OLA in an E. coli strain comprising fadD, and E. coli accABCD under an IPTG-inducible T7 promoter, OLS, OAC, and a deletion of fadE gene.

FIG. 9 is a bar graph showing the effect of deletion of E. coli nudB gene on the production of CBGA in an in an E. coli strain comprising OLS, OAC, GPP pathway genes (thiM, IPK, idi, and idsA), and deletion of nudB gene.

FIG. 10 is a bar graph showing the dephosphorylation of IPP to 3-methyl-3-butanol by various Nudix proteins.

FIG. 11 is a bar graph showing the effect of downregulation of fabD on the total OLA pathway flux in E. coli strains comprising ACC, fabD, OLS, and OAC. Strain 13883 is the parental control with wild type fabD and the various other strains having genotype as the parental strain 13883 with the exception that the strains have the RBS sequence modified to lower its protein expression (FabD60, FabD24, FabD41, FabD46, FabD22, FabD12, FabD28, FabD30, FabD5, FabD1, FabD23, FabD13).

FIG. 12 is a graph showing the proteomic analysis of effect of FabD ribosomal binding site (RBS) variation on the expression of FabD.

### DETAILED DESCRIPTION OF THE DISCLOSURE

The present invention provides engineered cells that make available more malonyl-CoA, one or more fatty acyl-CoAs, geranyldiphosphate (GPP), and/or produce one or more cannabinoids (or its derivatives), and methods for using and culturing the same. The engineered cells may have one or multiple modifications, including, without limitation, the downregulation, disruption, or deletion of one or more endogenous genes, the upregulation of one or more endogenous genes, and the introduction of one or more exogenous/heterologous genes, and combinations thereof.

The term “non-naturally occurring”, when used in reference to an organism (e.g., microbial) or host cell is intended to mean that the organism or host cell has at least one genetic alteration not normally found in a naturally occurring organism of the referenced species that is the result of human intervention. Naturally-occurring organisms can be referred to as “wild-type” such as wild type strains of the referenced species.

A genetic alteration that makes an organism or cell non-natural can include, for example, modifications introducing expressible nucleic acids encoding metabolic polypeptides, other nucleic acid additions, nucleic acid deletions and/or other functional disruption of the organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon.

A host cell, organism, or microorganism engineered to express or overexpress a gene, a nucleic acid, nucleic acid sequence, or nucleic acid molecule, or to overexpress an enzyme or polypeptide has been genetically engineered through recombinant DNA technology to include a gene or nucleic acid sequence that it does not naturally include that encodes the enzyme or polypeptide or to express an endogenous gene at a level that exceeds its level of expression in a non-altered cell. As non-limiting examples, a host cell, organism, or microorganism engineered to express or overexpress a gene, a nucleic acid, nucleic acid sequence, or nucleic acid molecule, or to overexpress an enzyme or polypeptide can have any modifications that affect a coding sequence of a gene, the position of a gene on a chromosome or episome, or regulatory elements associated with a gene. Overexpression of a gene can also be by increasing the copy number of a gene in the cell or organism. Similarly, a host cell, organism, or microorganism engineered to under-express (or to have reduced expression of) a gene, nucleic acid, nucleic acid sequence, or nucleic acid molecule, or to under-express an enzyme or polypeptide can have any modifications that affect a coding sequence of a gene, the position of a gene on a chromosome or episome, or regulatory elements associated with a gene. Specifically included are gene disruptions, which include any insertions, deletions, or sequence mutations into or of the gene or a portion of the gene that affect its expression or the activity of the encoded polypeptide. Gene disruptions include “knockout” mutations that eliminate expression of the gene. Modifications to under-express a gene also include modifications to regulatory regions of the gene that can reduce its expression

The term “exogenous” is intended to mean that the referenced molecule or the referenced activity is introduced into the host microbial organism. The molecule can be introduced, for example, by introduction of an encoding nucleic acid into the host genetic material such as by integration into a host chromosome or as non-chromosomal genetic material that may be introduced on a vehicle such as a plasmid. Therefore, the term as it is used in reference to expression of an encoding nucleic acid refers to introduction of the encoding nucleic acid in an expressible form into the microbial organism. When used in reference to a biosynthetic activity, the term refers to an activity that is introduced into the host reference organism. The source can be, for example, a homologous or heterologous encoding nucleic acid that expresses the referenced activity following introduction into the host microbial organism. Therefore, the term “endogenous” refers to a referenced molecule or activity that is naturally present in the host. Similarly, the term when used in reference to expression of an encoding nucleic acid refers to expression of an encoding nucleic acid contained within the microbial organism. The term “heterologous” refers to a molecule or activity derived from a source other than the referenced species whereas “homologous” refers to a molecule or activity derived from the host microbial 5 organism/species. Accordingly, exogenous expression of an encoding nucleic acid can utilize either or both of a heterologous or homologous encoding nucleic acid.

When used to refer to a genetic regulatory element, such as a promoter, operably linked to a gene, the term “homologous” refers to a regulatory element that is naturally operably linked to the referenced gene. In contrast, a “heterologous” regulatory element is not naturally found operably linked to the referenced gene, regardless of whether the regulatory element is naturally found in the host species.

It is understood that when more than one exogenous nucleic acid is included in a microbial organism, the more than one exogenous nucleic acid(s) refers to the referenced encoding nucleic acid or biosynthetic activity, as discussed above. It is further understood, as disclosed herein, that more than one exogenous nucleic acid(s) can be introduced into the host microbial organism on separate nucleic acid molecules, on polycistronic nucleic acid molecules, or a combination thereof, and still be considered as more than one exogenous nucleic acid/nucleic acid sequence. For example, as disclosed herein a microbial organism can be engineered to express at least two, three, four, five, six, seven, eight, nine, ten or more exogenous nucleic acids encoding a desired pathway enzyme or protein. In the case where two or more exogenous nucleic acids encoding a desired activity are introduced into a host microbial organism, it is understood that the two or more exogenous nucleic acids can be introduced as a single nucleic acid, for example, on a single plasmid, on separate plasmids, can be integrated into the host chromosome at a single site or multiple sites, and still be considered as two or more exogenous nucleic acids. Similarly, it is understood that more than two exogenous nucleic acids can be introduced into a host organism in any desired combination, for example, on a single plasmid, on separate plasmids, can be integrated into the host chromosome at a single site or multiple sites, and still be considered as two or more exogenous nucleic acids, for example three exogenous nucleic acids. Thus, the number of referenced exogenous nucleic acids or biosynthetic activities refers to the number of encoding nucleic acids or the number of biosynthetic activities, not the number of separate nucleic acids introduced into the host organism.

By “exogenous nucleic acid sequence” is meant a nucleic acid that is not naturally-occurring within the cell (e.g., a host cell) or organism. Exogenous nucleic acid sequence may be derived from or identical to a naturally-occurring nucleic acid sequence or it may be a heterologous nucleic acid sequence. For example, a duplication of a naturally-occurring gene is considered to be an exogenous nucleic acid sequence. In some embodiments, the exogenous nucleic acid sequence may be a heterologous nucleic acid sequence.

Genes or nucleic acid sequences can be introduced stably or transiently into a host cell using techniques well known in the art including, but not limited to, conjugation, electroporation, chemical transformation, transduction, transfection, and ultrasound transformation. Optionally, for exogenous expression in E. coli or other prokaryotic cells, some nucleic acid sequences in the genes or cDNAs of eukaryotic nucleic acids can encode targeting signals such as an N-terminal mitochondrial or other targeting signal, which can be removed before transformation into prokaryotic host cells, if desired. For example, removal of a mitochondrial leader sequence led to increased expression in E. coli (Hoffmeister et al., J. Biol. Chem. 280:4329-4338 (2005)). For exogenous expression in yeast or other eukaryotic cells, genes can be expressed in the cytosol without the addition of leader sequence, or can be targeted to mitochondrion or other organelles, or targeted for secretion, by the addition of a suitable targeting sequence such as a mitochondrial targeting or secretion signal suitable for the host cells. Thus, it is understood that appropriate modifications to a nucleic acid sequence to remove or include a targeting sequence can be incorporated into an exogenous nucleic acid sequence to impart desirable properties. Furthermore, genes can be subjected to codon optimization with techniques well known in the art to achieve optimized expression of the proteins.

The percent identity (% identity) between two sequences is determined when sequences are aligned for maximum homology, and not including gaps or truncations as set forth in the BLAST parameters. Exemplary parameters for determining relatedness of two or more amino acid sequences using the BLAST algorithm, for example, can be as provided in BLASTP using the following parameters: Matrix: 0 BLOSUM62; gap open: 11; gap extension: 1; x_dropoff: 50; expect: 10.0; wordsize: 3; filter: on. Nucleic acid sequence alignments can be performed using BLASTN and the following parameters: Match: 1; mismatch: −2; gap open: 5; gap extension: 2; x_dropoff: 50; expect: 10.0; wordsize: 11; filter: off. Those skilled in the art will know what modifications can be made to the above parameters to either increase or decrease the stringency of the comparison, for example, for determining the relatedness of two or more sequences. Additional sequences added to a polypeptide sequence, such as but not limited to immunodetection tags, purification tags, localization sequences (presence or absence), etc., do not affect the % identity.

Algorithms well known to those skilled in the art, such as Align, BLAST, Clustal W and others compare and determine a raw sequence similarity or identity, and also determine the presence or significance of gaps in the sequence which can be assigned a weight or score. Such algorithms also are known in the art and are similarly applicable for determining nucleotide or amino acid sequence similarity or identity, and can be useful in identifying orthologs of genes of interest. Parameters for sufficient similarity to determine relatedness are computed based on well-known methods for calculating statistical similarity, or the chance of finding a similar match in a random polypeptide, and the significance of the match determined. A computer comparison of two or more sequences can, if desired, also be optimized visually by those skilled in the art. Related gene products or proteins can be expected to have a high similarity, for example, 45% to 100% sequence identity. Proteins that are unrelated can have an identity which is essentially the same as would be expected to occur by chance if a database of sufficient size is scanned (about 5%).

For example, alignment can be performed using the Needleman-Wunsch algorithm (Needleman, S. & Wunsch, C. A general method applicable to the search for similarities in the amino acid sequence of two proteins J. Mol. Biol, 1970, 48, 443-453) implemented through the BALIGN tool (http://balign.sourceforge.net/). Default parameters are used for the alignment and BLOSUM62 was used as the scoring matrix. In some cases, it can be useful to use the Basic Local Alignment Search Tool (BLAST) algorithm to understand the sequence identity between an amino acid motif in a template sequence and a target sequence. Therefore, in preferred modes of practice, BLAST is used to identify or understand the identity of a shorter stretch of amino acids (e.g. a sequence motif) between a template and a target protein. BLAST finds similar sequences using a heuristic method that approximates the Smith-Waterman algorithm by locating short matches between the two sequences. The (BLAST) algorithm can identify library sequences that resemble the query sequence above a certain threshold.

A homolog is a gene or genes that are related by vertical descent and are responsible for substantially the same or identical functions in different organisms. Genes are related by vertical descent when, for example, they share sequence similarity of sufficient amount to indicate they are homologous or related by evolution from a common ancestor. Genes that are orthologous can encode proteins with sequence similarity of about 45% to 100% amino acid sequence identity, and more preferably about 60% to 100% amino acid sequence identity. Genes can also be considered orthologs if they share three-dimensional structure but not necessarily sequence similarity, of a sufficient amount to indicate that they have evolved from a common ancestor to the extent that the primary sequence similarity is not identifiable. Paralogs are genes related by duplication within a genome, and can evolve new functions, even if these are related to the original one.

An amino acid position (or simply, amino acid) “corresponding to” an amino acid position in another polypeptide sequence is the position that is aligned with the referenced amino acid position when the polypeptides are aligned for maximum homology, for example, as determined by BLAST which allows for gaps in sequence homology within protein sequences to align related sequences and domains. Alternatively, in some instances, when polypeptide sequences are aligned for maximum homology, a corresponding amino acid may be the nearest amino acid to the identified amino acid that is within the same amino acid biochemical grouping—i.e., the nearest acidic amino acid, the nearest basic amino acid, the nearest aromatic amino acid, etc. to the identified amino acid.

By “substantially identical,” with reference to a nucleic acid sequence (e.g., a gene, RNA, or cDNA) or amino acid sequence (e.g., a protein or polypeptide) is meant one that has at least 50%, at least 55%, at least 60%, at least 65%, at least 70%, at least 75%, at least 80%, at least 85%, at least 90%, at least 95%, at least 96%, at least 97% at least 98%, or at least 99% nucleotide or amino acid identity, respectively, to a reference sequence.

In some embodiments, the acyl-CoA substrate has the following structure:

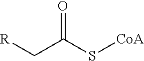

wherein R is a branched or linear alkyl side chain optionally comprising one or more functional and/or reactive groups as disclosed herein (i.e., an acyl-CoA compound derivative). In some embodiments, functional groups may include, but are not limited to, azido, halo (e.g., chloride, bromide, iodide, fluorine), alkynyl, alkenyl, methoxy, alkoxy, acetyl, amino, carboxyl, carbonyl, oxo, ester, hydroxyl, thio, cyano, aryl, heteroaryl, cycloalkyl, cycloalkenyl, cycloalkylalkenyl, cycloalkylalkynyl, cycloalkenylalkyl, cycloalkenylalkenyl, cycloalkenylalkynyl, heterocyclylalkenyl, heterocyclylalkynyl, heteroarylalkenyl, heteroarylalkynyl, arylalkenyl, arylalkynyl, heterocyclyl, spirocyclyl, heterospirocyclyl, thioalkyl, sulfone, sulfonyl, sulfoxide, amido, alkylamino, dialkylamino, acylamino, alkylarylamino, diarylamino, N-oxide, imide, enamine, imine, oxime, hydrazone, nitrile, aralkyl, cycloalkylalkyl, haloalkyl, heterocyclylalkyl, heteroarylalkyl, nitro, thioxo, and the like.

In some embodiments, the reactive groups may include, but are not necessarily limited to, azide, carboxyl, carbonyl, amine, (e.g., alkyl amine (e.g., lower alkyl amine), aryl amine), halide, ester (e.g., alkyl ester (e.g., lower alkyl ester, benzyl ester), aryl ester, substituted aryl ester), cyano, thioester, thioether, sulfonyl halide, alcohol, thiol, succinimidyl ester, isothiocyanate, iodoacetamide, maleimide, hydrazine, alkynyl, alkenyl, and the like. A reactive group may facilitate covalent attachment of a molecule of interest. Functional and reactive groups may be optionally substituted with one or more additional functional or reactive groups.

In some embodiments, the acyl-CoA substrate is selected from the group consisting of acetyl-CoA, propionyl-CoA, butyryl-CoA, valeryl-CoA, hexanoyl-CoA, heptanoyl-CoA, octanoyl-CoA, nonanoyl-CoA, and decanoyl-CoA. In some embodiments, the acyl chain may be C₂-C₆, C₂-C₈, C₂-C₁₀, C₂-C₂₀, C₆-C₁₀, C₆-C₂₀, or C₁₀-C₂₀.

CBGA Synthetic Pathway

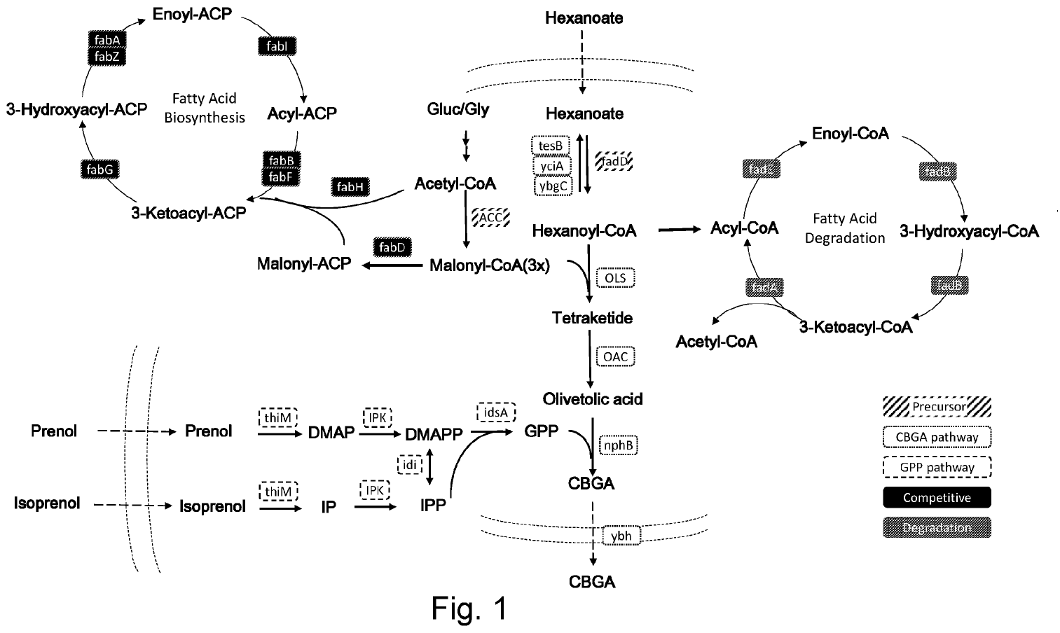

FIG. 1 is a schematic diagram illustrating the relevant synthetic pathways that may be engineered to improve the production of CBGA and its analogs and precursors. In relevant part, CBGA (or its analog) is produced from GPP and olivetolic acid (OA) (or its analog) by prenyltransferase. Cannabinoids (e.g., CBGA) or derivatives may be then secreted by the cell via a variety of known and putative cannabinoid transporters including ybhS, yhF, ybhR, and ybhG. The present inventions are based on engineered cells that have higher levels of available GPP and OA (and OA analogs or derivatives) for increased production of cannabinoids (e.g., CBGA) or derivatives and/or increased expression of cannabinoid transporters to effect increased efflux of cannabinoids (e.g., CBGA) or derivatives from the cell.

(A) Olivetolic Acid Production

As illustrated in FIG. 1, intracellular acyl carboxylic acid (e.g., hexanoate) is converted to acyl-CoA (e.g., Hex-CoA) by a fatty acyl-CoA ligase (e.g., fadD). One molecule of Hex-CoA (or other acyl-CoA) is combined with three molecules of malonyl-CoA (Mal-CoA) by olivetol synthase (OLS, also called 3,5,7-trioxododecanoyl-CoA synthase and tetraketide synthase, EC 2.3.1.206) or its variants to form a tetraketide (e.g., 3,5,7-trioxododecanoyl-CoA or 3,5,7-trioxododecanoic acid) which is subsequently converted to OA (or analogs thereof) by olivetolic acid cyclase (OAC, EC 4.4.1.26) or its variants. Although these metabolic pathways are illustrated with reference to certain precursors and intermediates, it is understood that analogs may be substituted in essentially the same reactions. For example, it is understood that hexanoate analogs including other carboxylic acids may be used in place of hexanoate. Suitable substitutions include, for example and without limitation, acetic acid, propionic acid, butyric acid, valeric acid (pentanoic acid), heptanoic acid, caprylic acid (octanoic acid), and generally any C₂-C₂₀ carboxylic acid. Similarly, it is understood that Hex-CoA is exemplary of the class of acyl-CoAs described above.

It is therefore desirable to increase the cell's production and availability of the precursors Mal-CoA and/or Hex-CoA (or other acyl-CoA) which often are limiting in the production of OA (or OA analogs). This may be achieved in two ways: by increasing the precursor production and by limiting precursor metabolism through competing (non-OA-producing) pathways. Engineered cells of the present invention comprise non-naturally-occurring modification that take advantage of either or both of these strategies in order to increase the production of OA (or OA analogs) and ultimately CBGA (or its associated analog). Methods for each of these is described in detail below.

It also is desirable to increase the production of OA (or OA analogs) directly, particularly in engineered cells in which the OA precursors (or OA precursor analogs), Mal-CoA and/or acyl-CoA, are produced in excess. Thus, in some embodiments, the cells are engineered to express an exogenous (e.g., a heterologous) or over-express an exogenous or endogenous OLS and/or OAC. In some embodiments, the OLS and OAC are non-naturally occurring OLS and OAC, respectively. In some embodiments, the OLS and/or OAC comprise one or more amino acid substitutions. In some embodiments, one or more amino acid substitutions of OLS and/or OAC increases the activity of the enzyme as compared to their naturally occurring counterpart.

Non-natural OAC variants are also described in commonly assigned International Application No. PCT/US2020/036310, filed Jun. 5, 2020 (Noble, et al.), the disclosure of which is incorporated herein by reference.

Olivetol synthase (OLS) belongs to plant type III polyketide synthases (PKS) which are a group of condensing enzymes that catalyze the initial key reactions in the biosynthesis of a myriad of secondary metabolites. All the plant type III polyketide synthases that have been characterized are homodimeric proteins. Each monomer of the dimeric protein contains its own active site and catalyzes the sequential condensation of starter CoA molecule and one acyl unit from malonyl-CoA, independently. Each condensation step is associated with one decarboxylation step. Olivetol synthases are classified as EC:2.3.1.206 under the Enzyme Commision nomenclature. Olivetol synthases have structural similarities with plant type III PKS enzymes. The OLS enzyme comprises conserved Cys157-His 297-Asn 330 catalytic triad, and the ‘gatekeeper’ Phe 208 corresponding to the amino acid positions of SEQ ID NO: 122. These amino acid residues are conserved for all other OLS homologs corresponding to SEQ ID Nos: 123-131. In some embodiments, the OLS has amino acid sequence that is substantially identical to any one of SEQ ID NOs: 122-131. In some embodiments, the amino acid sequence of the non-natural olivetol synthase has one or more amino acid variations at position(s) selected from the group consisting of: 125, 126, 185, 187, 190, 204, 209, 210, 211, 249, 250, 257, 259, 331, and 332 corresponding to the amino acid sequence of SEQ ID NO:122.

Although the positions recited herein are with reference to the corresponding amino acid sequence of SEQ ID NO:122, it is expressly contemplated that the amino acid sequence of the non-natural olivetol synthase can have one or more amino acid variations at equivalent positions (variant positions) corresponding to the homologs of SEQ ID NO: 122, e.g., SEQ ID NOs: 123-131. SEQ ID NOs 122-131 align very well and therefore identification of variant positions in any of SEQ ID NOs: 123-131 that correspond to variant positions in SEQ ID NO:122 can readily be understood.

In some embodiments, the amino acid substitutions designed to increase OA production by OLS are shown below. The amino acid positions of OLS corresponds to SEQ ID NO: 122. It is expressly contemplated that the amino acid sequence of the non-natural OLS can have one or more amino acid variations at equivalent positions corresponding to the homologs of SEQ ID NO: 122, e.g., SEQ ID Nos 123-131 (Table 1).

TABLE 1
Amino Acid Substitutions

| Position | Substitution |
|---|---|
| A125 | G, S, T, C, Y, H, N, Q, D, E, K, R |
| S126 | G, A |
| D185 | G, A, S, P, C, T, N |
| M187 | G, A, S, P, C, T, D, N, E, Q, H, V, L, I, K, R |
| L190 | G, A, S, P, C, T, D, N, E, Q, H, V, M, I, K, R |
| G204 | A, C, P, V, L, I, M, F, W |
| G209 | A, C, P, V |
| D210 | A, C, P, V |
| G211 | A, C, P, V |
| G249 | A, C, P, V, L, I, M, F, W, S, T, Y, H, N, Q, D, |
| | E, K, R |
| G250 | A, C, P, V, L, I, M, F, W, S, T, Y, H, N, Q, D, |
| | E, K, R |
| L257 | V, M, I, K, R, F, Y, W, S, T, C, H, N, Q, D, E |
| F259 | G, A, C, P, V, L, I, M, Y, W, S, T, Y, H, N, Q, |
| | D, E, K, R |
| M331 | G, A, S, P, C, T, D, N, E, Q, H, V, L, I, K, R |
| S332 | G, A |

For example, in some embodiments, in a non-natural OLS of the disclosure based on any one of SEQ ID NOs: 122-126, there can be an amino acid variant selected from G, S, T, C, Y, H, N, Q, D, E, K, or R at position 125, which replaces the wild type A. However, the corresponding position in SEQ ID NO: 128 is shifted +11, which corresponds to position 136. Since the wild type amino acid at position 136 in SEQ ID NO: 128 is already T, the amino acid variant can be selected from G, S, C, Y, H, N, Q, D, E, K, and R (i.e., excluding the wild-type T as a possibility) for position 136 to create a non-natural OLS. In embodiments wherein a single amino acid variant is prescribed at a certain amino acid position, but the prescribed substitution is already present as a wild type amino acid at that position, then another variant amino acid position is looked to so the non-natural OLS can be based on a non-wild type, prescribed variant, amino acid.

In some embodiments, the non-naturally-occurring OLS contains one or more of the following mutations relative to the corresponding to the amino acid positions of SEQ ID NO:122: A125G, A125S, A125T, A125C, A125Y, A125H, A125N, A125Q, A125D, A125E, A125K, A125R, S126G, S126A, D185G, D185G, D185A, D185S, D185P, D185C, D185T, D185N, M187G, M187A, M187S, M187P, M187C, M187T, M187D, M187N, M187E, M187Q, M187H, M187H, M187V, M187L, M187I, M187K, M187R, L190G, L190A, L190S, L190P, L190C, L190T, L190D, L190N, L190E, L190Q, L190H, L190V, L190M, L190I, L190K, L190R, G204A, G204C, G204P, G204V, G204L, G204I, G204M, G204F, G204W, G204S, G204T, G204Y, G204H, G204N, G204Q, G204D, G204E, G204K, G204R, G209A, G209C, G209P, G209V, G209L, G209I, G209M, G209F, G209W, G209S, G209T, G209Y, G209H, G209N, G209Q, G209D, G209E, G209K, G209R, D210A, D210C, D210P, D210V, D210L, D210I, D210M, D210F, D210W, D210S, D210T, D210Y, D210H, D210N, D210Q, D210E, D210K, D210R, G211A, G211C, G211P, G211V, G211L, G211I, G211M, G211F, G211W, G211S, G211T, G211Y, G211H, G211N, G211Q, G211D, G211E, G211K, G211R, G249A, G249C, G249P, G249V, G249L, G249I, G249M, G249F, G249W, G249S, G249T, G249Y, G249H, G249N, G249Q, G249D, G249E, G249K, G249R, G249S, G249T, G249Y, G250A, G250C, G250P, G250V, G250L, G250I, G250M, G250F, G250W, G250S, G250T, G250Y, G250H, G250N, G250Q, G250D, G250E, G250K, G250R, L257V, L257M, L257I, L257K, L257R, L257F, L257Y, L257W, L257S, L257T, L257C, L257H, L257N, L257Q, L257D, L257E, F259G, F259A, F259C, F259P, F259V, F259L, F259I, F259M, F259Y, F259W, F259S, F259T, F259Y, F259H, F259N, F259Q, F259D, F259E, F259K, F259R, M331G, M331A, M331S, M331P, M331C, M331T, M331D, M331N, M331E, M331Q, M331H, M331V, M331L, M331I, M331K, M331R, S332G, and S332A. It is understood that the foregoing mutations, while identified in the context of SEQ ID NO.: 122 may be made to the corresponding amino acids in an OLS of SEQ ID NOs: 123-131.

Non-natural OLS variants are also described in commonly assigned International Application No. PCT/US2020/028766, filed Apr. 17, 2020 (Noble, et al.), the disclosure of which is incorporated herein by reference.

In some embodiments, non-natural OLS with one or more variant amino acids as described herein are enzymatically capable of at least about 1.2, 1.5, 2, 3, 4, 5, 6, 7, 8, 9, 10, 12, 15, 20, or greater rate of formation of OA and/or olivetol from malonyl-CoA and hexanoyl-CoA in the presence of excess OAC enzyme, as compared to the wild type OLS.

In some embodiments, the OAC is present in molar excess of OLS. In some embodiments, the molar ratio of OLS to OAC is about 1:1.1, 1:1.2, 1:1.5, 1:1.8, 1:2, 1:3, 1:4, 1:5, 1:10, 1:20, 1:25, 1:50, 1:75, 1:100, 1:125, 1:150, 1:200, 1:250, 1:300, 1:350, 1:400, 1:450, 1:500, 1:1000, 1:1250, 1:1500, 1:2000, 1:2500, 1:5000, 1:7500, 1:10,000, or more. In some embodiments, the catalytic rate of OAC is greater than OLS. In some embodiments, the ratio of the catalytic rate of OLS to OAC is about 1:1.1, 1:1.2, 1:1.5, 1:1.8, 1:2, 1:3, 1:4, 1:5, 1:10, 1:20, 1:25, 1:50, 1:75, 1:100, 1:125, 1:150, 1:200, 1:250, 1:300, 1:350, 1:400, 1:450, 1:500, 1:1000, 1:1250, 1:1500, 1:2000, 1:2500, 1:5000, 1:7500, 1:10,000, or more.

For example, in the presence of excess OAC the increase in rate of formation of olivetolic acid from malonyl-CoA and hexanoyl-CoA, as compared to the wild olivetol synthase, can be in the range of about 1.2 times to about 300 times, about 1.5 times to about 200 times, or about 2 times to about 30 times as determined in an in vitro enzymatic reaction using purified olivetol synthase variant.

(i) Mal-CoA Availability

Availability of the Mal-CoA levels inside the cell may be increased by increasing the functional ACC activity within the cell. In some aspects, engineered cells of the present invention express higher ACC activity compared to host cells. The elevated ACC activity may be achieved using one or more of the following strategies/modifications: (i) overexpress one or more (i.e., one, two, three, four, or more) endogenous ACC subunit proteins (e.g., under the control of a heterologous promoter); (ii) expressing one or more (i.e., one, two, three, four, or more) exogenous (e.g., heterologous) ACC subunit proteins; (iii) expressing one or more multi-domain proteins having ACC activity (e.g., that are identical or substantially identical to the fungal ACC genes described herein); and (iv) expressing one or more non-naturally-occurring proteins that have an activity associated with one of the ACC subunit proteins or the activity associated with a multi-domain ACC protein. Each of these strategies/modifications is described in more detail below.

Acetyl-CoA Carboxylase (ACC; EC 6.4.1.2) catalyzes the committed step in fatty acid biosynthesis in carboxylating acetyl-CoA to produce malonyl-CoA. Malonyl-CoA also is a key substrate for cannabinoid formation, where three malonyl-CoA molecules are successively condensed with an acyl-CoA molecule (e.g., hexanoyl-CoA) to form a tetraketide (e.g., 3,5,7-trioxododecanoyl-CoA or 3,5,7-trioxododecanoic acid). In prokaryotes (e.g., E. coli) and the chloroplasts of plants and algae, ACC typically is a multi-subunit enzyme. The typical prokaryotic (E. coli) ACC has four polypeptide subunits: accA (EC 2.1.3.15), accB (EC 6.4.1.2), accC (EC 6.3.4.14), and accD (EC 2.1.3.15) that assemble into a complex having three functional subunits: a tetramer made up of two homodimers of the biotin carboxylase subunit (BC, P_417722, encoded by accC, in E. coli), a heterotetramer of two of each of the accA and accD subunits (P_414727 and P_416819) forming the carboxytransferase (CT), and the biotin carboxylase carrier protein (BCCP, encoded by accB, P_417721 in E. coli) (see Broussard et al. (2013) Biochemistry 14:3346-3357). In prokaryotes, biotin protein ligase (BPL, encoded by BirA in E. coli) covalently attaches biotin to the carboxylase subunit; this function is performed by a holocarboxylase synthetase in eukaryotes. Thus, one or more of the endogenous ACC subunit proteins may be overexpressed using an exogenous (e.g., a heterologous) promoter. Alternatively, a cell may be engineered to express one or more the ACC subunit proteins from a different species under the control of an endogenous promoter or an exogenous (e.g., heterologous) promoter. Alternatively, an expression system (e.g., vector) may be constructed in which one, two, three, or all four of the ACC subunit proteins are expressed as a single polypeptide joined directly or through a polypeptide linker. For example, if all four ACC subunit proteins were linked and expressed as a single protein, the result would mimic the fungal multi-domain proteins described below.

Fungal ACC genes encode large single chain polypeptides having multiple active domains which, together, have ACC activity. The multi-domain proteins, including the fungal ACC genes, generally have three conserved catalytic domains contained within the protein. The catalytic domains, arranged in N-to-C order, include the biotin carboxylase (BC) domain, the biotin-carboxyl carrier protein (BCCP) domain, and the carboxyltransferase (CT) domain.

The BC domain contains an ATP binding site which, in many instances, has an amino acid sequence that is identical or substantially identical to:
G(F/Y)P(V/L)MIKASEGGGGKGIR(M/K/Q)(V/A) (SEQ ID NO: 118)

The BCCP domain contains a biotin binding site which, in many instances, has an amino acid sequence that is identical or substantially identical to: EVMKM (SEQ ID NO: 119). Additionally, the BCCP domain frequently contains two proline residues located about 26 and 34 residues upstream from the biotin binding site. These proline residues may for a hinge region.

The CT domain contains binding sites for carboxybiotin and acetyl-CoA. These binding sites generally conform to the structures found in other carboxylase enzymes. For example, a carboxybiotin binding site may be identical or substantially identical to:
GR(X)₆NDIT(X)₂IGSFG(X)₂ED(X)₇E(L/Y)AR(X)₂GIRR(IN)YL(A/S)ANSGARI
(SEQ ID NO: 120).
   The acetyl-CoA binding site may identical or substantially identical to:
A(R/K)(T/G)VVVGRARLGGIP(M/L/V)GV(I/V)(S/A/G)
(SEQ ID NO: 121).

The fungal ACC genes may be under the control of a promoter and regulatory sequences from the host cell, the fungal cell from which the ACC gene is derived, or another heterologous promoter. The engineered cells expressing the fungal ACC gene(s) may retain endogenous expression of the native ACC gene(s) and subunits, may be engineered to overexpress the native ACC gene(s) and subunits, or may have expression of one or more of the native ACC gene(s) and subunits reduced or eliminated (knocked out). Thus, in some embodiments, one or more nucleic acid sequences encoding a fungal ACC or derivative thereof is overexpressed in a host cell.

Suitable fungal ACC enzymes include the ACC from Mucor circinelloides f. circinelloides strain 1006PhL (SEQ ID NO: 1) and other fungal proteins having substantial amino acid sequence identity and similar enzymatic activity. One nucleic acid sequence encoding the Mucor circinelloides f. circinelloides strain 1006PhL ACC protein is provided at SEQ ID NO: 101. Table 2 provides examples of suitable fungal ACC enzymes that may be used in the engineered cells provided herein. Considered for use in the engineered cells provided herein are single domain protein ACC homologs of other species, and nucleic acids encoding the same, as well as variants of naturally-occurring single domain protein ACCs having at least 50%, at least 55%, at least 60%, at least 65%, at least 70%, at least 75%, at least 80%, at least 85%, at least 90%, at least 95%, at least 96%, at least 97% at least 98%, or at least 99% amino acid identity to SEQ ID NO: 1 and that have the enzymatic activity of an ACC.

TABLE 2
Fungal Acetyl-CoA Carboxylases

| | GenBank | SEQ ID |
|---|---|---|
| Species | Accession No. | NO: |
| Mucor circinelloides f. circinelloides | EPB82652.1 | 1 |
| 1006PhL | | |
| Amylomyces rouxii | ABQ28729.1 | 2 |
| Mucor circinelloides f. lusitanicus CBS | OAD07937.1 | 3 |
| 277.49 | | |
| Mucor ambiguus | GAN08941.1 | 4 |
| Parasitella parasitica | CEP09288.1 | 5 |
| Rhizopus stolonifer | RCI05537.1 | 6 |
| Choanephora cucurbitarum | OBZ91864.1 | 7 |
| Rhizopus delemar RA 99-880 | EIE80272.1 | 8 |
| Rhizopus microsporus ATCC 52813 | XP_023470433.1 | 9 |
| Rhizopus microsporus | ORE23244.1 | 10 |
| Rhizopus microspores | CEG63811.1 | 11 |
| Rhizopus azygosporus | RCH96152.1 | 12 |
| Rhizopus microspores | CEI99071.1 | 13 |
| Absidia repens | ORZ25473.1 | 14 |
| Absidia glauca | SAL97607.1 | 15 |
| Phycomyces blakesleeanus NRRL 1555(-) | XP_018296012.1 | 16 |
| Hesseltinella vesiculosa | ORX57605.1 | 17 |
| Rhizopus stolonifera | EPB82652.2 | 18 |
| Lichtheimia ramose | ABQ28729.2 | 19 |
| Lichtheimia ramose | OAD07937.2 | 20 |
| Lichtheimia corymbifera JMRC:FSU:9682 | GAN08941.2 | 21 |
| Absidia glauca | CEP09288.2 | 22 |
| Syncephalastrum racemosum | RCI05537.2 | 23 |
| Mucor circinelloides f. circinelloides | OBZ91864.2 | 24 |
| 1006PhL | | |
| Mucor circinelloides f. lusitanicus CBS | EIE80272.2 | 25 |
| 277.49 | | |
| Mucor ambiguous | XP_023470433.2 | 26 |
| Lichtheimia corymbifera JMRC:FSU:9682 | ORE23244.2 | 27 |
| Bifiguratus adelaidae | CEG63811.2 | 28 |
| Endogone sp. FLAS-F59071 | RCH96152.2 | 29 |
| Glomus cerebriforme | CEI99071.2 | 30 |
| Jimgerdemannia flammicorona | ORZ25473.2 | 31 |
| Lobosporangium transversal | SAL97607.2 | 32 |
| Rhizophagus irregularis | XP_018296012.2 | 33 |
| Rhizophagus irregularis | ORX57605.2 | 34 |
| Rhizophagus irregularis | EPB82652.3 | 35 |
| Rhizophagus irregularis | ABQ28729.3 | 36 |
| Mortierella elongata AG-77 | OAD07937.3 | 37 |
| Mortierella verticillata NRRL 6337 | GAN08941.3 | 38 |
| Diversispora epigaea | CEP09288.3 | 39 |
| Rhizophagus irregularis | RCI05537.3 | 40 |
| Rhizophagus clarus | OBZ91864.3 | 41 |
| Basidiobolus meristosporus CBS 931.73 | EIE80272.3 | 42 |
| Gigaspora rosea | XP_023470433.3 | 43 |
| Rhizophagus irregularis | ORE23244.3 | 44 |
| Rhizophagus diaphanus [Rhizophagus sp. | CEG63811.3 | 45 |
| MUCL 43196] | | |
| Basidiobolus meristosporus CBS 931.73 | RCH96152.3 | 46 |
| Saitoella complicata NRRL Y-17804 | CEI9907L3 | 47 |
| Saitoella complicata NRRL Y-17804 | ORZ25473.3 | 48 |
| Basidiobolus meristosporus CBS 931.73 | SAL97607.3 | 49 |
| Coleophoma cylindrospora | XP_018296012.3 | 50 |
| Dactylellina haptotyla CBS 200.50 | ORX57605.3 | 51 |
| Pyronema omphalodes CBS 100304 | EPB82652.4 | 52 |
| Aspergillus fischeri NRRL 181 | ABQ28729.4 | 53 |
| Coleophoma crateriformis | OAD07937.4 | 54 |
| Aspergillus turcosus | GAN08941.4 | 55 |
| Aspergillus lentulus | CEP09288.4 | 56 |
| Byssochlamys spectabilis | RCI05537.4 | 57 |
| Aspergillus fumigatus A1163 | OBZ91864.4 | 58 |
| Aspergillus udagawae | EIE80272.4 | 59 |
| Aspergillus thermomutatus | XP_023470433.4 | 60 |
| Aspergillus fumigatus Af293 | ORE23244.4 | 61 |
| Botrytis tulipae | CEG63811.4 | 62 |
| Morchella conica CCBAS932 | RCH96152.4 | 63 |
| Botrytis cinerea B05.10 | CEI99071.4 | 64 |
| Aspergillus fumigatus var. RP-2014 | ORZ25473.4 | 65 |
| Botrytis galanthina | SAL97607.4 | 66 |
| Botrytis elliptica | XP_018296012.4 | 67 |
| Byssochlamys spectabilis No. 5 | ORX57605.4 | 68 |
| Xylona heveae TC161 | EPB82652.5 | 69 |
| Botrytis cinerea BcDW1 | ABQ28729.5 | 70 |
| Phialocephala scopiformis | OAD07937.5 | 71 |
| Sclerotinia sclerotiorum 1980 UF-70 | GAN08941.5 | 72 |
| Amorphotheca resinae ATCC 22711 | CEP09288.5 | 73 |
| Aspergillus oryzae RIB40 | RCI05537.5 | 74 |
| Aspergillus parasiticus SU-1 | OBZ91864.5 | 75 |
| Phialocephala subalpine | EIE80272.5 | 76 |
| Rutstroemia sp. NJR-2017a WRK4 | XP_023470433.5 | 77 |
| Rutstroemia sp. NJR-2017a BVV2 | ORE23244.5 | 78 |
| Aspergillus costaricaensis CBS 115574 | CEG63811.5 | 79 |
| Pezoloma ericae | RCH96152.5 | 80 |
| Aspergillus brasiliensis CBS 101740 | CEI99071.5 | 81 |
| Aspergillus vadensis CBS 113365 | ORZ25473.5 | 82 |
| Aspergillus heteromorphus CBS 117.55 | SAL97607.5 | 83 |
| Aspergillus piperis CBS 112811 | XP_018296012.5 | 84 |
| Rutstroemia sp. NJR-2017a BBW | ORX57605.5 | 85 |
| Drechslerella stenobrocha 248 | EPB82652.6 | 86 |
| Botrytis hyacinthi | ABQ28729.6 | 87 |
| Aspergillus bombycis | OAD07937.6 | 88 |
| Glonium stellatum | GAN08941.6 | 89 |
| Botryotinia convolute | CEP09288.6 | 90 |
| Aspergillus clavatus NRRL 1 | RCI05537.6 | 91 |
| Aspergillus neoniger CBS 115656 | OBZ91864.6 | 92 |
| Erysiphe pulchra | EIE80272.6 | 93 |
| Penicillium chrysogenum | XP_023470433.6 | 94 |
| Penicillium rubens Wisconsin 54-1255 | ORE23244.6 | 95 |
| Botrytis paeoniae | CEG63811.6 | 96 |
| Glarea lozoyensis ATCC 20868 | RCH96152.6 | 97 |
| Aspergillus eucalypticola CBS 122712 | CEI99071.6 | 98 |
| Pseudogymnoascus sp. VKM F-4515 | ORZ25473.6 | 99 |
| (FW-2607) | | |
| Aspergillus niger CBS 513.88 | SAL97607.6 | 100 |
| Saccharomyces cerevisiae | AAA20073 | 208 |
| Yarrowia lipolytica | VBB85319 | 209 |

In some embodiments, increasing the malonyl CoA availability inside the engineered cell improves the ratio of Olivetolic acid/PDAL as compared a control cell with identical genotype exception that the control cell does not have increased malonyl-CoA availability inside the cell.

(ii) Mal-CoA Degradation

Mal-CoA is a common intracellular precursor for a wide array of biomolecules and processes. In order to increase Mal-CoA availability for the OA synthetic pathway, and ultimately CBGA production, it may be desirable to inhibit (i.e., reduce or completely block) competing Mal-CoA metabolic pathways. As illustrated in FIG. 1, fatty acid biosynthesis utilizes a significant amount of intracellular Mal-CoA in a competitive pathway that does not result in cannabinoid production. Thus, limiting fatty acid biosynthesis can increase the Mal-CoA supply available for cannabinoid biosynthesis. In some embodiments, the engineered cell has one or more genes of a fatty acid biosynthetic pathway downregulated, deleted, or disrupted. The following discussion illustrates certain principles of the invention and fatty acid biosynthesis genes that may be disrupted however, these examples should not be taken as limiting. The invention includes the disruption or downregulation of any one or more genes known to be involved in any fatty acid biosynthetic pathway that consumes Mal-CoA.

In some embodiments, the engineered cells have a downregulation, deletion, or disruption of the malonyl-CoA-ACP transacylase gene (FabD; EC 2.3.1.39) which catalyzes the transfer of a the malonyl moiety from Mal-CoA to an acyl carrier protein (ACP) with the concomitant release of the free CoA. This is the first step in the fatty acid biosynthetic pathway and represents a significant source of Mal-CoA consumption. In an E. coli host cell, for example, the FabD gene and protein are homologs to or have sequences that are substantially identical to those of the E. coli K-12 MG1655 FabD and provided at SEQ ID NO: 102 (see, for example, GenBank Accession No. NP 415610.1).

In some embodiments, the engineered cells have a downregulation, deletion, or disruption of the 3-hydroxyacyl-ACP dehydratase (FabZ; EC 4.2.1.59) which catalyzes the dehydration of 3-hydroxyacyl-ACP to enoyl-ACP. This is an intermediate step in the fatty acid biosynthetic pathway. Disruption of FabZ alone or in combination with disruption of FabD reduces or eliminates Mal-CoA consumption by this pathway. In an E. coli host cell, for example, the FabZ gene and protein are homologs to or have sequences that are substantially identical to those of the E. coli K-12 MG1655 FabZ and provided at SEQ ID NO: 103 (see, for example, GenBank Accession No. NP 414722.1).

Host cells that are engineered for increased malonyl-CoA supply also can include genetic modifications such as, but not limited to, downregulation, including disruption, of genes encoding enzymes that may reduce the supply of acetyl-CoA and/or malonyl-CoA available for OA or cannabinoid production, such as but not limited to alcohol dehydrogenase, lactate dehydrogenase, phosphate acetyl transferase, acetate kinase, succinate dehydrogenase, or citrate synthase. Downregulation of one or more genes encoding fatty acid biosynthesis enzymes (e.g., in prokaryotic hosts FabH, FabB, FabF, FabG, FabA, and/or FabI).

(iii) Acyl-CoA Production

As shown in FIG. 1, acyl-CoA (e.g., Hex-CoA) is condensed with three molecules of Mal-CoA by OLS (or another Type III PKS) to generate a tetraketide (e.g., 3,5,7-trioxododecanoyl-CoA or 3,5,7-trioxododecanoic acid) which is subsequently converted to OA (or an analog thereof). In some embodiments, in E. coli, the fatty acid CoA ligase, fadD (EC 6.2.1.3), catalyzes the esterification of fatty acids into metabolically active CoA thioesters. In particular, fadD or a variant of fadD is responsible for catalyzing the reaction between hexanoic acid and CoA to produce Hex-CoA. Thus, the host cells may be engineered to overexpress endogenous fadD or a variant of fadD or another enzyme in the EC 6.2.1.3 class, or express or overexpress a heterologous fadD or EC 6.2.1.3 class enzyme. In some embodiments, the variant of fadD is a non-naturally occurring variant of fadD. In some embodiments, the fadD variant comprises one or more amino acid substitutions. One example of E. coli fadD protein, obtained from E. coli K-12 MG1656, is found at NCBI Accession No. NP 416319.1 and provided as SEQ ID NO: 104. Also included within the invention are naturally-occurring and non-naturally-occurring homologs of the fadD protein based on the same of different species from the host cell, and nucleic acids encoding the same. Such variant proteins have at least 50%, at least 55%, at least 60%, at least 65%, at least 70%, at least 75%, at least 80%, at least 85%, at least 90%, at least 95%, at least 96%, at least 97% at least 98%, or at least 99% amino acid identity to SEQ ID NO: 104 and that have the enzymatic activity of EC 6.2.1.3. Engineered cells which express a heterologous fadD or EC 6.2.1.3 class enzyme may retain endogenous expression of the native fadD gene, may be engineered to overexpress the native fadD gene, or may have expression of the native fadD gene reduced or eliminated (knocked out).

(iv) Acyl-CoA Degradation

Acyl-CoA (e.g., Hex-CoA) is a common intracellular precursor for a wide array of biomolecules and processes. In order to increase acyl-CoA (e.g., Hex-CoA) availability for the OA synthetic pathway, and ultimately cannabinoid production, it may be desirable to inhibit (i.e., reduce or completely block) competing acyl-CoA (e.g., Hex-CoA) metabolic pathways. In some embodiments, the engineered cells have a downregulation, deletion, or disruption of one or more of the thioesterase genes that degrade Hex-CoA. In E. coli, such genes include, for example, TesB (NP 414986.1; acyl-CoA thioesterase II; EC 3.1.2.20; SEQ ID NO: 107), YciA (NP 415769.1; acyl-CoA thioesterase; EC 3.1.2.20; SEQ ID Ns: 108), and YbgC (NP 415264.1; acyl-CoA esterase/thioesterase; SEQ ID NO: 109), ydiI, tesA, and fadM. It is understood that endogenous homologs and other endogenous enzymes that have substantially the same catalytic activity may be downregulated, deleted, or disrupted and that the precise identity of these homologs depends upon the specific host cell type.

As illustrated in FIG. 1, certain fatty acid degradation reactions utilize a significant amount of intracellular acyl-CoA (e.g., Hex-CoA) pathway in a competitive pathway that does not result in cannabinoid production. Thus, limiting these competing fatty acid degradation reactions can increase the acyl-CoA (e.g., Hex-CoA) supply available for cannabinoid biosynthesis. In some embodiments, the engineered cell has one or more genes of a fatty acid degradation pathway downregulated, deleted, or disrupted. In E. coli, for example, the fadE gene (NP_414756.2; acyl-CoA dehydrogenase; EC 1.3.8.1; SEQ ID NO: 105) can be downregulated, disrupted, or deleted. It is understood that endogenous homologs and other endogenous enzymes that have substantially the same catalytic activity may be downregulated, deleted, or disrupted and that the precise identity of these homologs depends upon the specific host cell type.

Alternatively, or in addition to the downregulation, disruption, or deletion offadE, the engineered cell may have downregulation, disruption, or deletion in another gene associated with fatty acid degradation. In E. coli another suitable gene is fadB (NP_418288; enoyl-CoA hydratase; EC 4.2.1.17; SEQ ID NO: 106) which catalyzes the formation of 3-oxoacyl-CoA from enoyl-CoA via 3-hydroxyacyl-CoA. It is understood that endogenous homologs and other endogenous enzymes that have substantially the same catalytic activity may be downregulated, deleted, or disrupted and that the precise identity of these homologs depends upon the specific host cell type. In some embodiments, E. coli fadA gene can be downregulated, disrupted or deleted.

GPP Synthetic Pathway

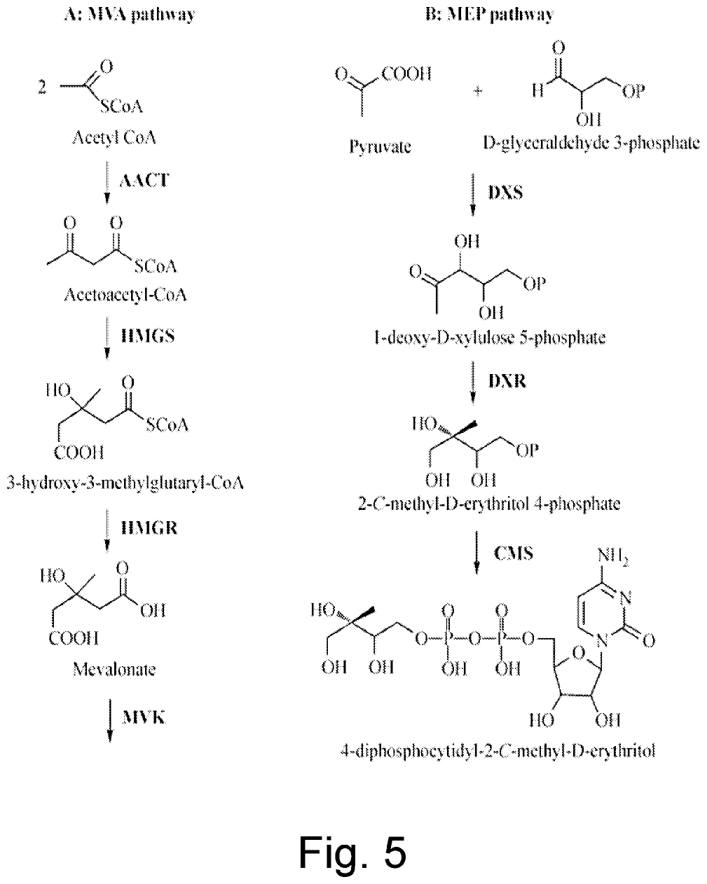

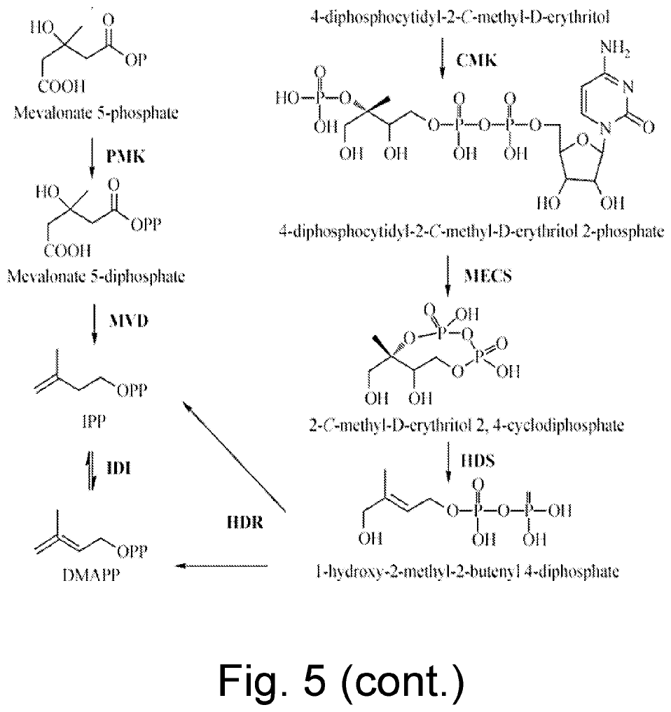

GPP and its precursors may be produced from several pathways within a host cells including the mevalonate pathway (MVA) or methylerythritol-4-phosphate (MEP) pathway (also known as the deoxyxylulose-5-phosphate pathway), which produce isopentenyl pyrophosphate (IPP) and dimethylallyl pyrophosphate (DMAPP), which are converted to geranyl pyrophosphate (GPP) using geranyl pyrophosphate synthase. A prenyltransferase converts OLA or its analogs or derivatives and GPP to a cannabinoid (e.g., CBGA, the common precursor to cannabinoids). Exemplary MVA and MEP pathways are shown in FIG. 5. The result of both the MVA pathway and the MEP pathway are the GPP precursors IPP and DMAPP which, as described in more detail below, may be isomerized through the action of, for example, the idi gene product, and combined, for example, through the action of the idsA gene product. Expression of an exogenous (e.g., heterologous) or overexpression of an endogenous gene that encodes any one or more of the enzymes in the MVA and/or MEP pathways increases the production of GPP and, ultimately, that of CBGA (or its analogs).

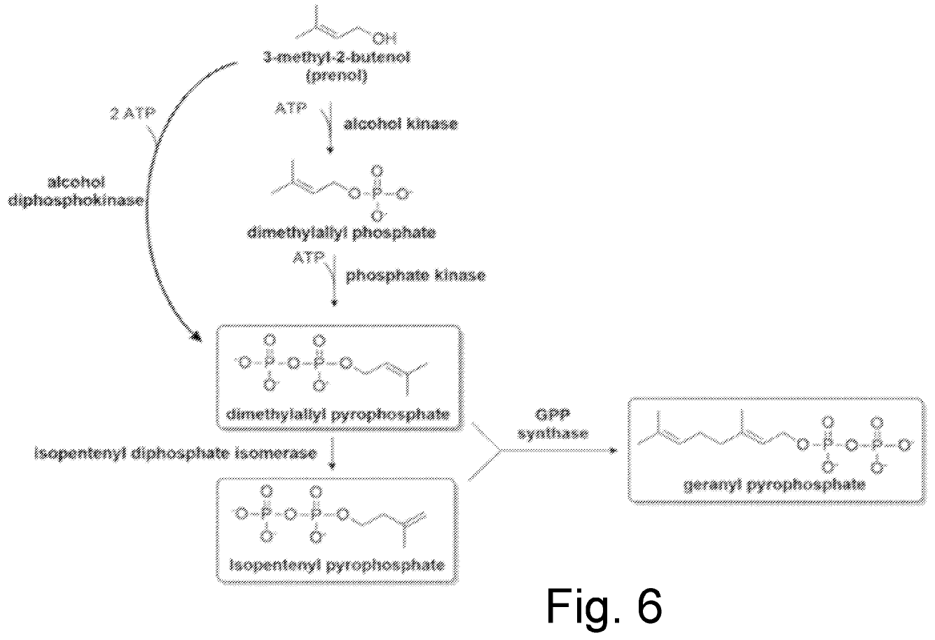

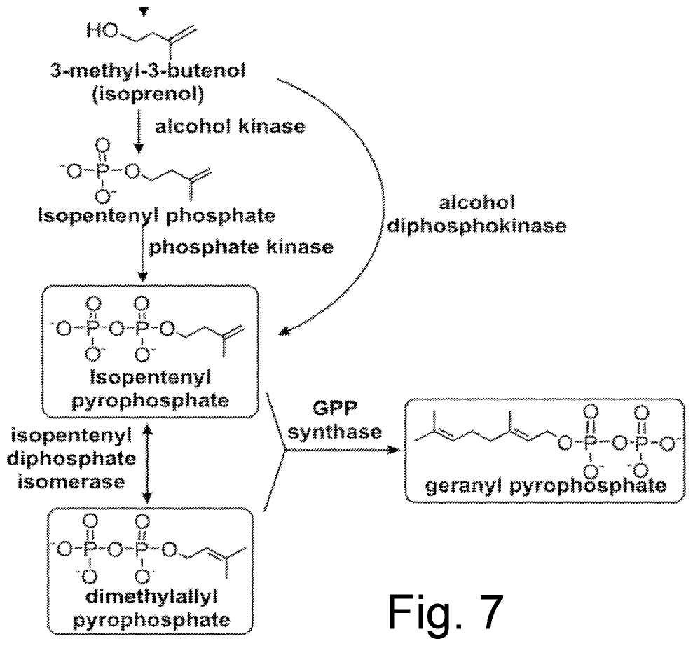

FIG. 1 illustrates two alternative GPP synthetic pathways from prenol and isoprenol. In the non-MVA pathway prenol is phosphorylated to dimethylallyl phosphate (DMAP) and then to dimethylallyl pyrophosphate (DMAPP) or directly to DMAPP. Phosphorylation of DMAP to DMAPP is catalyzed by isopentenyl phosphate kinase (IPK). Phosphorylation of prenol to DMAPP is catalyzed by prenol diphosphokinase. DMAPP and isopentenyl pyrophosphate (IPP) may be isomerized. GPP is synthesized from DMAPP and IPP in a reaction catalyzed by a GPP synthase. The non-MVA, non-MEP pathways are illustrated with additional detail in FIG. 6 and FIG. 7. Expression of an exogenous (e.g., heterologous) or overexpression of an endogenous gene that encodes any one or more of the enzymes in the non-MVA, non-MEP pathways increases the production of GPP and, ultimately, that of CBGA (or its analogs).

In the non-MEP, non-MVA pathways, isoprenol is phosphorylated to isopentenyl phosphate (IP) and then to to IPP or directly to isopentenyl pyrophosphate (IPP). Similarly, prenol is phosphorylated to dimethyl allyl pyrophosphate which can be converted to IPP. The non-MVA, non-MEP pathway has a common synthetic conclusion in that IPP is isomerized to DMAPP and/or combined with DMAPP to yield GPP. Expression of an exogenous (e.g., heterologous) or overexpression of an endogenous gene that encodes any one or more of the enzymes in the non-MEP pathway increases the production of GPP and, ultimately, that of CBGA.

In some embodiments, the host cells may be engineered to overexpress endogenous IPK or another enzyme in the EC 2.7.4.26 class, or express or overexpress a heterologous IPK or EC 2.7.4.26 class enzyme. One example of IPK, obtained from Methanothermobacter thermautotrophicus, is found at NCBI Accession No. WP_010875687.1 which is provided as SEQ ID NO: 110. Also included within the invention are naturally-occurring and non-naturally-occurring homologs of the IPK protein based on the same of different species from the host cell, and nucleic acids encoding the same. Such variant proteins have at least 50%, at least 55%, at least 60%, at least 65%, at least 70%, at least 75%, at least 80%, at least 85%, at least 90%, at least 95%, at least 96%, at least 97% at least 98%, or at least 99% amino acid identity to SEQ ID NO: 110 and that have the enzymatic activity of EC 2.7.4.26. Engineered cells which express a heterologous IPK or EC 2.7.4.26 class enzyme may retain endogenous expression of the native IPK gene, may be engineered to overexpress the native IPK gene, or may have expression of the native IPK gene reduced or eliminated (knocked out). Other enzymes that have a similar activity to IPK may be substituted for or used in addition to the IPK described herein. One class of such enzymes includes the choline kinases (EC 2.7.1.32).

In other embodiments, the host cells may be engineered to overexpress endogenous GPP synthase or another enzyme in the EC 2.5.1.- (e.g., EC 2.5.1.1) class, or express or overexpress a heterologous GPP synthase or EC 2.5.1.-class enzyme. Suitable GPP synthases for these embodiments include, for example, E. coli IspA (NP_414955, SEQ ID NO:111) and C. glutamicum IdsA (WP 011014931.1, SEQ ID NO:112). Also included within the invention are naturally-occurring and non-naturally-occurring homologs of GPP synthase based on the same of different species from the host cell, and nucleic acids encoding the same. Such variant proteins have at least 50%, at least 55%, at least 60%, at least 65%, at least 70%, at least 75%, at least 80%, at least 85%, at least 90%, at least 95%, at least 96%, at least 97% at least 98%, or at least 99% amino acid identity to SEQ ID NO: 111 and that have the enzymatic activity of EC 2.5.1.-. Engineered cells which express a heterologous GPP synthase or EC 2.5.1.-class enzyme may retain endogenous expression of the native GPP synthase gene, may be engineered to overexpress the native GPP synthase gene, or may have expression of the native GPP synthase gene reduced or eliminated (knocked out). Other GPP synthases that may be expressed or overexpressed in the engineered cells of the present invention include those provided in Table 3 and GPP synthases that are substantially identical (i.e., at least 95% identical) to the GPP synthases of Table 3. Other enzymes that have a similar activity to GPP synthase may be substituted for or used in addition to the GPP synthase described herein including, for example, the farnesyl pyrophosphate synthases (EC 2.5.1.10; “FPP synthases”) and the geranylgeranyl pyrophosphate synthases (EC 2.5.1.1; “GGPP synthases”).

TABLE 3
GPP Synthase Enzymes

| | Gen Bank | SEQ ID |
|---|---|---|
| Species | Accession No. | NO: |
| Corynebacterium crudilactis | WP_074025495.1 | 152 |
| Corynebacterium glutamicum | WP_096457048.1 | 153 |
| Corynebacterium deserti | WP_053545301.1 | 154 |
| Corynebacterium callunae | WP_015651699.1 | 155 |
| Corynebacterium efficiens | WP_006768068.1 | 156 |
| Corynebacterium sp. Marseille-P2417 | WP_080794061.1 | 157 |
| Corynebacterium humireducens | WP_040086238.1 | 158 |
| Corynebacterium halotolerans | WP_015401326.1 | 159 |
| Corynebacterium marinum | WP_042621772.1 | 160 |
| Corynebacterium singulare | WP_042531577.1 | 161 |
| Corynebacterium minutissimum | WP_115022907.1 | 162 |
| Corynebacterium pollutisoli | WP_143337494.1 | 163 |
| Corynebacterium lubricantis | WP_018297093.1 | 164 |
| Corynebacterium spheniscorum | WP_092284621.1 | 165 |
| Corynebacterium doosanense | WP_018020857.1 | 166 |
| Corynebacterium flavescens | WP_075731219.1 | 167 |
| Corynebacterium aurimucosum | WP_143334899.1 | 168 |
| Corynebacterium ammoniagenes | WP_003845210.1 | 169 |
| Corynebacterium keflrresidentii | WP_086587718.1 | 170 |
| Corynebacterium camporealensis | WP_035105251.1 | 171 |
| Corynebacterium tuberculostearicum | WP_005328932.1 | 172 |
| Corynebacteri um pseudogenitalium | WP_005324491.1 | 173 |
| Corynebacterium testudinoris | WP_083985528.1 | 174 |
| Corynebacterium stationis | WP_066793135.1 | 175 |
| Corynebacterium sp. J010B-136 | WP_105324112.1 | 176 |
| Corynebacterium sp. CCUG 69366 | WP_123047545.1 | 177 |
| Corynebacterium sp. KPL1818 | WP_023030480.1 | 178 |
| Corynebacterium accolens | WP_005283903.1 | 179 |
| Corynebacterium segmentosum | WP_126319428.1 | 180 |
| Corynebacterium macginleyi | WP_121911356.1 | 181 |
| Pseudomonas aeruginosa | SQG59150.1 | 182 |
| Streptococcus thermophilus | VDG63248.1 | 183 |
| Nocardia vermiculata | WP_084473733.1 | 184 |
| Rhodococcus sp. 1168 | WP_088945631.1 | 185 |
| Clostridium paraputrifleum | WP_113570111.1 | 186 |
| Nocardia cyriacigeorgica | WP_036535265.1 | 187 |
| Nocardia concava | WP_040806894.1 | 188 |
| Rhodococcus yunnanensis | WP_072806331.1 | 189 |

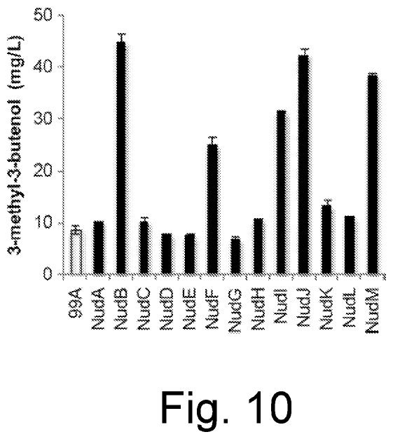

A complementary modification that may be introduced into the engineered cells in order to increase GPP production is the deletion, disruption, or downregulation of one or more Nudix hydrolases (EC 3.6.1.19). These enzymes act on a high diversity of substrates having a general structure consisting of a nucleoside diphosphate (“NDP”) linked to another moiety (“X”). The Nudix hydrolyases catalyze the hydrolysis of a pyrophosphate bond in a nucleoside diphosphate linked to some other moiety, “x”. The general hydrolase reaction cleaves NDP-X to NMP and P-X. Additionally, some Nudix proteins have been shown to dephosphorylate IPP, DMAPP, and GPP. For example, NudA, NudB, NudC, NudD, NudE, NudF, NudG, NudH, Nudl, NudJ, NudK, NudL, NudM dephosphorylate GPP, IPP, and/or dimethyl allyl pyrophosphate at varying degrees. FIG. 10 shows the dephosphorylation of IPP to 3-methyl-3-butanol by various Nudix proteins. In particular, the downregulation or disruption of one or more Nudix hydrolases in the engineered cell blocks a significant degradation pathway for several important metabolites including, for example, isopentyl pyrophosphate (IPP), dimethylallyl pyrophosphate (DMAPP), and geranyl pyrophosphate (GPP).

CBGA Production

CBGA is formed by the following reaction:

geranyl diphosphate+2,4-dihydroxy-6-pentylbenzoate [symbol] diphosphate+CBGA which is catalyzed by a geranyl-pyrophosphate-olivetolic acid geranyltransferase (EC 2.5.1.102). The enzyme carrying out the above reaction in C. sativa is a transmembrane prenyltransferase belonging to the UbiA superfamily of membrane proteins. See for example CsPT1 as described in WO2011017798A1 and CsPT4, as described in WO2018200888 and WO20190300888. However, the above reaction has also been reported to be carried out by a different family of enzymes. In particular, aromatic prenyltransferases that are soluble, non-transmembrane, and have a 10-stranded antiparallel β-barrel consisting of 5 repeated αββα motifs, can catalyze the transfer of isoprenoid chains to aromatic rings. For example, Yang, Y., et al. (Biochemistry 51:2606-180, 2012) reports that NphB, a Streptomyces-derived, soluble enzyme, catalyzes the attachment of a 10-carbon geranyl group to aromatic substrates; originally identified in the biosynthetic pathway of the antioxidant naphterpin. Yang notes the reaction mechanism of the prenylation step has been characterized as a S(N)1 type dissociative mechanism with a weakly stable carbocation intermediate. NphB catalyzes the prenyl transfer between GPP and 1,6 dihydroxynaphthalene (1,6-DHN) and yields three products with the geranyl moiety attaching to different carbon atoms of 1,6-DHN. The major product 5 geranyl DHN and minor product 2-geranyl DHN were characterized with a product ratio of 10:1. However, Kumano, et al. (Bioorg. Med. Chem, 16, 8117-8126 (2008)), reports rates and regioselectivity measurements for NphB-catalyzed geranylation of olivetol, with mixed regioselectivity at 2- and 4-OL ring positions, and rates of 0.0026 mol 2-geranyl-OL/min/mol NphB and 0.0016 mol 4-geranyl-OL/min/mol NphB, which are extremely slow.

Thus, the cells of the present invention may be engineered to overexpress a naturally-occurring prenyltransferase (e.g., nphB) or express or overexpress an exogenous (e.g., heterologous) prenyltransferase such that the production of a cannabinoid (e.g., CBGA) (or derivatives thereof) is increased relative to the host cells. Generally, the disclosure provides non-natural prenyltransferases that can form prenylated alkylbenzenediols or prenylated dihydroxyalkylbenzenoic acids from a substrate comprising a hydrophobic portion such as geranyl pyrophosphate, and alkylbenzenediols or dihydroxyalkylbenzenoic acids, respectively, at increased enzymatic rates as compared to wild-type versions of the enzymes. For example, the disclosure provides non-natural prenyltransferases that are enzymatically capable of greater rates of formation of 3-geranyl-olivetolate (3-GOLA; cannabigerolic acid; CBGA) from geranyl pyrophosphate and olivetolic acid, and/or that are enzymatically capable of greater rates of formation of cannabigerorcinic acid (CBGOA) from orsellinic acid (OSA) and geranyl diphosphate (GPP). Further, non-natural prenyltransferases of the disclosure with these increased enzymatic rates also demonstrate regioselectivity towards desired products, for example, the variants are capable of regioselectivity (e.g., about 90% or greater, about 95% or greater) to 2-prenylated, 5-alkylbenzene-1,3-diol or 3-prenylated, 2,4-dihydroxy 6-alkylbenzenoic acid from geranyl pyrophosphate and a 5-alkylbenzene-1,3-diol, or a 2,4-dihydroxy 6-alkylbenzenoic acid.

Non-natural prenyltransferase variants of the disclosure include those based on previously identified non-natural prenyltransferase variants already demonstrating improved enzymatic activity and desired regioselectivity over the wild type prenyltransferase. In particular, a non-natural prenyltransferase triple variant (Seq1C) used for generation of further variants is described in commonly assigned International Application No. PCT/US2019/021448 (filed Mar. 8, 2019; Noble, M.), wherein the triple Seq1C variant is based on SEQ ID NO:1 having Q159S, S212H, and Y286V variant amino acids. Enzyme activity of the Seq1C triple variant was shown to be >300-fold greater for conversion of OLA to CBGA, and >100-fold greater for conversion of OSA to CBGOA over the wild type prenyltransferase enzyme (SEQ ID NO: 1).

In some embodiments, the prenyltransferase comprises the amino acid sequence set forth in any one of SEQ ID NOs: 132-146 or that is substantially identical to SEQ ID NOs: 132-146, provided that the enzyme exhibits prenyltransferase activity. SEQ ID NO: 132 provides the amino acid sequence of the prenyltransferase of Streptomyces antibioticus AQJ23_40425 and is used as the reference sequence for numbering the amino acids when referring to various homologs and/or mutations. (Also see, International Application No. PCT/US2019/021448; hereby incorporated by reference in its entirety.) In some embodiments, the non-naturally-occurring prenyltransferase comprises one or more (e.g., two, three, four, five, six, seven, eight, or more) of the following mutations, with reference to SEQ ID NO: 132, or at the corresponding amino acid locations in SEQ ID NOs: 133-146: 5S, 17T, 25V, 38G, 45I; 45T; 45S, 49T; 51T, 51C, 51D, 51E, 51F, 51G, 51H, 51I, 51K, 51L, 51M, 51N, 51P, 51Q, 51R, 51S, 51T, 51V, 51W, 51Y; 62A; 78V; 80S; 104E; 106G; 110D, 110G; 116N, 116Q; 116A, 116D; 119W; 121V, 121A, 121L, 121K, 121H, 121W; 124K; 124L; 124S; 159S, 159A, 159C, 159D, 159E, 159F, 159G, 159H, 159I, 159K, 159L, 159M, 159P, 159R, 159T, 159V, 159W, 159Y; 160L; 160V; 160I; 164E; 171D; 172V; 173D; 173K; 173P; 173Q; 173E; 173F; 175W, 175E, 175Y; 203L, 203M; 207G; 211M 212F, 212R, 212A, 212C, 212D, 212D, 212E, 212G, 212K, 212L, 212M, 212P, 212Q, 212V, 212T, 212V, 212W, 212Y, 212H; 212N; 214A; 217F; 224A; 225E; 226E, 226Q; 228N, 228S; 230S; 232K; 232N; 232R; 232S; 232T; 241A; 257A; 267A; 267W, 267P; 268Y; 269E, 269F; 272V; 281L; 286A, 286C, 286D, 286E, 286F, 286G, 286G, 286H, 286H, 286I, 286K, 286L, 286M, 286N, 286P, 286Q, 286R, 286T, 286V; 286W; 288H; 290A; 2901; 290M; 290S; 292A, 292F, 292N; 293A, 293W, 293C, 293D, 293E, 293F, 293G, 293G, 293H, 293I, 293K, 293L, 293M, 293N, 293P, 293R, 293S, 293T, 293V, 293W; 294K; 294H; 296I; 296K; 296M; 296Q; 300I; 300P; 300Y; and 303V. Preferably, those non-naturally-occurring prenyltransferases are substantially identical to one or more of SEQ ID NOs: 132-146.

Optional additional mutations relative to SEQ ID NO: 132, or at the corresponding amino acid locations in SEQ ID NOs: 133-146, include: 47S, 47N, 47G; 121L; 161R, 161H, 161S; 175H, 175K, 175R; 211H; 211N; 214H; 230S; 268Y; 269N; 284S; 285Y; 286F, 286L, 286M, 286P, 286T, 286V, 286I, 286A; 288V, 288I; 296I; 293H, 293M, 293F, 293W, 293C, 293C, 293A, 293S, 293V, 293D, 293Y, 293E, 293I, and 293T.

In some embodiments, the non-naturally-occurring prenyltransferase comprises the following mutations relative to SEQ ID NO: 132, or at the corresponding amino acid locations in SEQ ID NOs: 133-146: (i) 45I, (ii) 159S, and (iii) 286V; (i) 45T, (ii) 159S, and (iii) 286V; (i) 121V, (ii) 159S, and (iii) 286V; (i) 124K, (ii) 159S, and (iii) 286V; (i) 124L, (ii) 159S, and (iii) 286V; (i) 159S, (ii) 160L, and (iii) 286V; (i) 159S, (ii) 160L, and (iii) 286V; (i) 159S, (ii) 160S, and (iii) 286V; (i) 159S, (ii) 173D, and (iii) 286V; (i) 159S, (ii) 173K, and (iii) 286V; (i) 159S, (ii) 173P, and (iii) 286V; (i) 159S, (ii) 173Q, and (iii) 286V; (i) 159S, (ii) 173Y, and (iii) 286V; (i) 159S, (ii) 212H, and (iii) 286V; (i) 159S, (ii) 230S, and (iii) 286V; (i) 159S, (ii) 267P, and (iii) 286V; (i) 159S, (ii) 286V, and (iii) 293H; (i) 159S, (ii) 286V, and (iii) 294K; (i) 159S, (ii) 286V, and (iii) 296K; (i) 159S, (ii) 286V, and (iii) 296L; (i) 159S, (ii) 286V, and (iii) 296M; (i) 159S, (ii) 286V, and (iii) 296Q; (i) 159S, (ii) 286V, and (iii) 296M; (i) 159S, (ii) 286V, and (iii) 300F; and (i) 159S, (ii) 286V, and (iii) 300Y.

In some embodiments, the non-naturally-occurring prenyltransferase comprises the following mutations relative to SEQ ID NO: 132, or at the corresponding amino acid locations in SEQ ID NOs: 133-146: (i) 45I, (ii) 159S, (iii) 212H, and (iv) 286V; (i) V45T, (ii) 159S, (iii) 212H, and (iv) 286V; (i) 121V, (ii) 159S, (iii) 212H, and (iv) 286V; (i) 124K, (ii) 159S, (iii) 212H, and (iv) 286V; (i) 124L, (ii) 159S, (iii) 212H, and (iv) 286V; (i) 159S, (ii) 160L, (iii) 212H, and (iv) 286V; (i) 159S, (ii) 160L, (iii) 212H, and (iv) 286V; (i) 159S, (ii) 160S, (iii) 212H, and (iv) 286V; (i) 159S, (ii) 173D, (iii) 212H, and (iv) 286V; (i) 159S, (ii) 173K, (iii) 212H, and (iv) 286V; (i) 159S, (ii) 173P, (iii) 212H, and (iv) 286V; (i) 159S, (ii) 173Q, (iii) 212H, and (iv) 286V; (i) 159S, (ii) 173Y, (iii) 212H, and (iv) 286V; (i) 159S, (ii) 212H, (iii) 213V, and (iv) 286V; (i) 159S, (ii) 212H, (iii) 230S, and (iv) 286V; (i) 159S, (ii) 212H, (iii) 267P, and (iv) 286V; (i) 159S, (ii) 212H, (iii) 286V, and (iv) 293H; (i) 159S, (ii) 212H, (iii) 286V, and (iv) 294K; (i) 159S, (ii) 212H, (iii) 286V, and (iv) 296K; (i) 159S, (ii) 212H, (iii) 286V, and (iv) 296L; (i) 159S, (ii) 212H, (iii) 286V, and (iv) 296M; (i) 159S, (ii) 212H, (iii) 286V, and (iv) 296Q; (i) 159S, (ii) 212H, (iii) 286V, and (iv) 296M; (i) 159S, (ii) 212H, (iii) 286V, and (iv) 300F; and (i) 159S, (ii) 212H, (iii) 286V, and (iv) 300Y.

In some embodiments, non-natural prenyltransferases with one or more variant amino acids as describe herein are enzymatically capable of a greater rate of formation of cannabigerolic acid from geranyl pyrophosphate and olivetolic acid, as compared to the wild type prenyltransferase. Variants were also identified that displayed very high activity on the order of about 300 fold or greater rate of formation of cannabigerolic acid from geranyl pyrophosphate and olivetolic acid, as compared to the wild type prenyltransferase. For example, the increase in rate of formation of cannabigerolic acid from geranyl pyrophosphate and olivetolic acid, as compared to the wild type prenyltransferase, can be in the range of about 1.5× to about 750×, about 5× to about 750×, or about 10× to about 750× as determined in an in vitro enzymatic reaction using purified prenyltransferase variant.

Using a purified prenyltransferase preparation the rate of formation of CBGA can be determined. The rate can be expressed in terms of μM CBGA/min/μM enzyme. Reaction conditions can be as follows: 50 mM HEPES, pH 7.5 buffer containing 1 mM geranyl pyrophosphate (Sigma-Aldrich) and 1 mM olivetolic acid (Santa Cruz Biotechnology) and 5 mM magnesium chloride. Reactions are initiated by addition of purified prenyltransferase and then incubated for a measured period of 0.5 to 2 hours, quenched with acetonitrile to a final concentration of 65%, then centrifuged to pellet denatured protein. Supernatants are transferred to 96-well plates for GCMS analysis of CBGA (3-GOLA) and 5-GOLA.

In embodiments, the prenyltransferase variants provide a rate of formation of CBGA of greater than 0.005 μM CBGA/min/μM enzyme, greater than about 0.010 μM CBGA/min/μM enzyme, greater than about 0.020 μM CBGA/min/μM enzyme, greater than about 0.050 μM CBGA/min/μM enzyme, greater than about 0.100 μM CBGA/min/μM enzyme, greater than about 0.250 μM CBGA/min/μM enzyme, greater than about 0.500 μM CBGA/min/μM enzyme, such as in the range of about 0.005 μM or 0.010 μM to about 1.250 μM CBGA/min/μM enzyme, or in the range of about 0.020 μM to about 1.0 μM CBGA/min/μM enzyme.

Improving Cannabinoid or Derivatives Efflux

The efficiency of the cannabinoid (or derivatives thereof) synthetic pathway may be improved by increasing the cannabinoid (or derivatives thereof) efflux from the engineered cell so that the various synthetic reactions do not become product-limited. By continually and efficiently depleting the reaction products (i.e., cannabinoid or derivatives thereof), all reactions are driven in the forward direction and reactants/precursors are less likely to be diverted to alternate (non-cannabinoid) pathways.

In some embodiments, the cannabinoid is CBGA, THCV, THCVA, CBDV, CBDVA, CBN, CBNA, CBD, CBDA, CBC, CBCA, CBGV, CBGVA, CBG, CBCV, CBCVA, THC, THCA, analogs, or derivatives thereof, or combinations thereof.

It has been discovered that cannabinoid (or derivatives thereof) efflux may be increased by overexpressing one or more genes of the endogenous ybh operon and/or the entire operon itself, or expressing one or more exogenous (e.g., a heterologous) ybh genes or operon. The ybh operon consists of ybiH, ybhG, ybhF, ybhS, and ybhR. All of these genes encode putative integral membrane or membrane transport proteins except ybiH, which is a transcriptional regulator. The predicted gene products comprise the subunits of an ATP-binding cassette (ABC) superfamily membrane transporter and membrane fusion protein, indicating that this operon encodes the components of a transport complex spanning the inner and outer membranes of E. coli.

In some embodiments, the host cells may be engineered to overexpress one or more endogenous cannabinoid (or derivatives thereof) transporters and accessory proteins, or express or overexpress one or more heterologous CBGA transporters and accessory proteins. Examples of cannabinoid (or derivatives thereof) transporters obtained from E. coli K-12 MG1656 include ybhS (NP_415314.1; multidrug ABC transporter permease; EC 7.6.2.2; SEQ ID NO: 113), ybhF (NP_415315.2; multidrug ABC transporter ATP-binding protein; EC 7.6.2.2; SEQ ID NO: 114), ybhR (NP_415313.1; EC 7.6.2.2; multidrug ABC transporter permease; SEQ ID NO: 115), and ybhG (NP_415316.1; UPF0194 membrane protein; EC 7.6.2.2; SEQ ID NO: 116). Other proteins involved in cannabinoid (or derivatives thereof) efflux that may be overexpressed and/or expressed exogenously in engineered cells include blc (NP_418573; SEQ ID NO: 147), ydhC (YP_025306; SEQ ID NO: 148), mlaD (NP_417660.1; SEQ ID NO: 149), mlaE (NP_417661.1; SEQ ID NO: 150), and mlaf (NP_417662.1; SEQ ID NO: 151), and EmrB/QacA subfamily drug resistance transporters, such as pur8 proteins, including SEQ ID NO: 210 (A0A0F7N8B6); SEQ ID NO: 211 (A0A0F7NJP6); and SEQ ID NO: 212 (A0A0F7N8B6); and SEQ ID NO: 213 (2775244747), and SEQ ID NO: 214 (2515835837). The following identifiers/accessions numbers are provided SEQ ID NO: 210 (AA: A0A0F7N8B6; NA: 2654644352, Ga0081730_111627); SEQ ID NO: 211 (AA:A0A0F7NJP6; NA: 2654644361, Ga0081730_111636); SEQ ID NO: 212 (AA: A0A0F7N8B6; NA: 2561472617, T413DRAFT_02996), SEQ ID NO: 213 (NA: 2775244747, Ga0198854_112262), and SEQ ID NO: 214 (NA: 2515835837, B100DRAFT_06500). Other ABC-type transporters include the following genes from E. coli: msbA (UniProt P60752; SEQ ID NO: 215), macAB, (UniProt P75830, P75831; SEQ ID NO: 216), mdlAB (UniProt P77265, P0AAG5; SEQ ID NO: 217), yadGH (UniProt P36879, P0AFN6; SEQ ID NO: 218), ybbAP (UniProt P0A9T8, P77504; SEQ ID NO: 219), yddA (UniProt P31826; SEQ ID NO: 220), yojI (UniProt P33941; SEQ ID NO: 221), and yhhJ (UniProt P0AGH1; SEQ ID NO: 222). ABC-type transporters in E. coli are discussed in Moussatova et al. (Biochim Biophys Acta 1778:1757-71, 2008).

In some embodiments, the host cell is engineered to express one or more exogenous nucleic acid sequences or overexpress one or more endogenous genes encoding a protein having a resistance-nodulation-cell division (RND) transporter. As discussed Anes et al. (Frontiers Microbiol, 6:587, 2015), and with reference to Nikaido, H. (Adv. Enzymol. Relat. Areas Mol. Biol. 77,1-60, 2011) resistance-nodulation-cell division transporters operate as part of a tripartite system composed of the RND pump located in the inner membrane, a periplasmic adaptor protein from the MFP family and an OMP belonging to the outer membrane factor (OMF) family located in the outer membrane. Exemplary RND-type transporters include the following genes from E. coli: acrAB (Uniprot P0AE06, P31224; SEQ ID NO: 223). acrEF (Uniprot P24180, P24181; SEQ ID NO: 224), mdtABC (Uniprot P76397, P76398, P76399; SEQ ID NO: 225), mdtEF (Uniprot P37636, P37637; SEQ ID NO: 226), emrAB (Uniprot P27303, P0AEJ0; SEQ ID NO: 227), and tolC (Uniprot P02930; SEQ ID NO: 228).

In some embodiments, the host cell is engineered to express one or more exogenous nucleic acid sequences or overexpress one or more endogenous genes encoding a protein having a prokaryotic small multidrug (SMR) transporter. As discussed in Jack, D. L., et al. (Eur. J. Biochem. 268, 3620-3639 (2001)) SMR family pumps are prokaryotic transport systems consisting of homo-oligomeric or heterooligomeric structures, with subunits of 100-120 aminoacyl residues in length and that span the membrane as a helices four times. An exemplary SMR transporter includes the following gene from E. coli: emrE (UniProt P23895; SEQ ID NO: 229).

In some embodiments, the host cell is engineered to express one or more exogenous nucleic acid sequences or overexpress one or more endogenous genes encoding a protein that is a member of the major facilitator superfamily (DIES). As described in Yan, (Ann. Rev. Biophys. 44:257, 2015) MPS proteins transport a broad spectrum of ions and solutes across membranes via facilitated diffusion, symport, or antiport. Exemplary MFS-type transporters include the following genes from E. coli: mdtM (Uniprot P39386; SEQ ID NO: 230) and mdfA (Uniprot P0AEY8; SEQ ID NO: 231).

In some embodiments the engineered cell expresses (a) exogenous nucleic acid sequences encoding (a1) olivetol synthase, (a2) olivetolic acid cyclase, (a3) prenyltransferase, and (a4) one or more genes of a MVA pathway, MEP pathway, or a non-MVA, non-MEP pathway; and (b) one or more of the following: (b1) a multi-domain acetyl-CoA carboxylase (MD-ACC), overexpress acetyl-CoA carboxyltransferase subunit α, biotin carboxyl carrier protein, biotin carboxylase, and/or acetyl-CoA carboxyltransferase subunit β, or expresses acetyl-CoA carboxyltransferase, biotin carboxyl carrier protein, and/or biotin carboxylase, (b2) a fatty acyl-CoA ligase, disruption or downregulation of fatty acid beta-oxidation repressor or fatty acid biosynthesis upregulator, 3-oxoacyl-[acyl-carrier-protein] synthase, enoyl-[acyl-carrier-protein] reductase, and (b3) ABC transporter permease; ABC transporter ATP-binding protein; a protein with at least 60% identity to: the blc gene product of SEQ ID NO: 147, ybhG gene product of SEQ ID NO: 116, or the ydhC gene product of SEQ ID NO: 148, or EmrB/QacA subfamily drug resistance transporters, such as the pur8 protein, of one of SEQ ID NOs: 210-214, or expresses one or more exogenous nucleic acids sequences or overexpress one or more endogenous genes that encodes a protein that is at least 60% identical to the mlaD gene product of SEQ ID NO: 149, the mlaE gene product of SEQ ID NO: 150, the mlaF gene product of SEQ ID NO: 151, or the RND family MdtABC.

In some embodiments the engineered cell expresses: (a1)-(a4) and (b1); (a1)-(a4) and (b2); (a1)-(a4) and (b3); (a1)-(a4), (b1) and (b2); (a1)-(a4), (b1) and (b3); (a1)-(a4), (b2) and (b3); (a1)-(a4) and (b1)-(b3). In some embodiments, (b1) is expression of acetyl-CoA carboxyltransferase (ACC), such as C. glutamicum or M. circinelloides ACC, or a protein having >60%, ≥65%, ≥70%, ≥75%, ≥80%, ≥85%, ≥90%, ≥95%, ≥97%, ≥99%, or 100% identity to said sequences. In some embodiments, (b2) is deletion, disruption, or reduced expression of one or more of fabA, fabB, fabD, fabF, fabG, fabH, fabL, fadE, fadD, fadI, fadM, fadL, and fadR. In some embodiments (b3) is expression of one or more of blc, ybhG, ydhC, mlaD, mlaE, mlaF, or MdtABC.

Also included within the invention are naturally-occurring and non-naturally-occurring homologs of cannabinoid (or derivatives thereof) transporters based on the same of different species from the host cell, and nucleic acids encoding the same. Such variant proteins have at least 50%, at least 55%, at least 60%, at least 65%, at least 70%, at least 75%, at least 80%, at least 85%, at least 90%, at least 95%, at least 96%, at least 97% at least 98%, or at least 99% amino acid identity to any of the foregoing. Engineered cells which express a heterologous cannabinoid (or derivatives thereof) transporter, accessory protein, or EC 7.6.2.2 class enzyme/protein may retain endogenous expression of the native cannabinoid (or derivatives thereof) transporter, accessory protein, or EC 7.6.2.2 class enzyme/protein, may be engineered to overexpress the native cannabinoid (or derivatives thereof) transporter, accessory protein, or EC 7.6.2.2 class enzyme/protein, or may have expression of the native cannabinoid (or derivatives thereof) transporter gene, accessory protein gene, or EC 7.6.2.2 class enzyme/protein gene reduced or eliminated (knocked out).

The transcriptional regulator ybiH (cecR) (NP_415317.1; SEQ ID NO: 117), isolated from E. coli K-12 MG1656, is an HTH-type transcriptional dual regulator down-regulates the expression of certain endogenous cannabinoid (or derivatives thereof) transporters and accessory proteins. Thus, in some embodiments, host cells are engineered to have ybiH downregulated, deleted, or disrupted in order to increase the expression of one or more endogenous cannabinoid (or derivatives thereof) transporters or accessory proteins, and/or to increase the expression of one or more heterologous cannabinoid (or derivatives thereof) transporters or accessory proteins that are regulated under the same or similar promoters. Although ybiH is identified in E. coli K-12 MG1656, it is understood that endogenous homologs and other endogenous transcriptional regulators that have substantially the same activity may be downregulated, deleted, or disrupted and that the precise identity of these homologs depends upon the specific host cell type.

Host Cells

A host cell as provided herein can be a prokaryotic cell or a eukaryotic cell. Eukaryotic cells may be microbial eukaryotic cells, such as, for example, fungal cells or microalgal cells. Further, a eukaryotic cell engineered to produce at least one cannabinoid can be a cell or cell line derived from a multicellular eukaryote, such as but not limited to an alga, moss, or higher plant. Prokaryotic cells that can be engineered as provided herein include bacterial cells, archaebacterial cells, and cyanobacterial cells.

In some embodiments, a host cell is a microorganism such as a bacterium, filamentous fungus, or yeast. Host can be selected based on their ability to take up and utilize particular carbon sources, nitrogen sources, or precursor molecules or may be engineered to take up and utilize molecules that may be added to the culture medium.

Nonlimiting examples of suitable microbial hosts for the bio-production of a cannabinoid include, but are not limited to, any Gram negative organisms, more particularly a member of the family Enterobacteriaceae, such as E. coli, or Oligotropha carboxidovorans, or a Pseudomononas sp.; any Gram positive microorganism, for example Bacillus subtilis, Lactobaccilus sp. or Lactococcus sp.; a yeast, for example Saccharomyces cerevisiae, Pichia pastoris or Pichia stipitis; and other groups or microbial species. More particularly, suitable microbial hosts for the bio-production of cannabinoids generally include, but are not limited to, members of the genera Clostridium, Zymomonas, Escherichia, Salmonella, Rhodococcus, Pseudomonas, Bacillus, Lactobacillus, Enterococcus, Alcaligenes, Klebsiella, Paenibacillus, Arthrobacter, Corynebacterium, Brevibacterium, Pichia, Candida, Hansenula, and Saccharomyces. Hosts that may be particularly of interest include: Oligotropha carboxidovorans (such as strain OM5), Escherichia coli, Alcaligenes eutrophus (Cupriavidus necator), Bacillus licheniformis, Paenibacillus macerans, Rhodococcus erythropolis, Pseudomonas putida, Lactobacillus plantarum, Enterococcus faecium, Enterococcus gallinarium, Enterococcus faecalis, Bacillus subtilis and Saccharomyces cerevisiae.

A variety of microorganism may be suitable for the production of cannabinoids in cell culture. Such organisms include both prokaryotic and eukaryotic organisms including, but not limited to, bacteria, including archaea and eubacteria, and eukaryotes, including yeast, plant, insect, animal, and mammal, including human. Exemplary species are reported in U.S. application Ser. No. 13/975,678 (filed Aug. 26, 2013), which is incorporated herein by reference, and include, for example, Escherichia coli, Saccharomyces cerevisiae, Saccharomyces kluyveri, Candida boidinii, Clostridium kluyveri, Clostridium acetobutylicum, Clostridium beijerinckii, Clostridium saccharoperbutylacetonicum, Clostridium perfringens, Clostridium difficile, Clostridium botulinum, Clostridium tyrobutyricum, Clostridium tetanomorphum, Clostridium tetani, Clostridium propionicum, Clostridium aminobutyricum, Clostridium sub terminale, Clostridium sticklandii, Ralstonia eutropha, Mycobacterium bovis, Mycobacterium tuberculosis, Porphyromonas gingivalis, Arabidopsis thaliana, Thermus thermophilus, Pseudomonas species, including Pseudomonas aeruginosa, Pseudomonas putida, Pseudomonas stutzeri, Pseudomonas fluorescens, Homo sapiens, Oryctolagus cuniculus, Rhodobacter spaeroides, Thermoanaerobacter brockii, Metallosphaera sedula, Leuconostoc mesenteroides, Chloroflexus aurantiacus, Roseiflexus castenholzii, Erythrobacter, Simmondsia chinensis, Acinetobacter species, including Acinetobacter calcoaceticus and Acinetobacter baylyi, Porphyromonas gingivalis, Sulfolobus tokodaii, Sulfolobus solfataricus, Sulfolobus acidocaldarius, Bacillus subtilis, Bacillus cereus, Bacillus megaterium, Bacillus brevis, Bacillus pumilus, Rattus norvegicus, Klebsiella pneumonia, Klebsiella oxytoca, Euglena gracilis, Treponema denticola, Moorella thermoacetica, Thermotoga maritima, Halobacterium salinarum, Geobacillus stearothermophilus, Aeropyrum pernix, Sus scrofa, Caenorhabditis elegans, Corynebacterium glutamicum, Acidaminococcus fermentans, Lactococcus lactis, Lactobacillus plantarum, Streptococcus thermophilus, Enterobacter aerogenes, Candida, Aspergillus terreus, Pedicoccus pentosaceus, Zymomonas mobilus, Acetobacter pasteurians, Kluyveromyces lactis, Eubacterium barkeri, Bacteroides capillosus, Anaerotruncus colihominis, Natranaerobius thermophilusm, Campylobacter jejuni, Haemophilus influenzae, Serratia marcescens, Citrobacter amalonaticus, Myxococcus xanthus, Fusobacterium nuleatum, Penicillium chrysogenum, marine gamma proteobacterium, butyrate producing bacterium, Nocardia iowensis, Nocardia farcinica, Streptomyces griseus, Schizosaccharomyces pombe, Geobacillus thermoglucosidasius, Salmonella typhimurium, Vibrio cholera, Heliobacter pylori, Nicotiana tabacum, Oryza sativa, Haloferax mediterranei, Agrobacterium tumefaciens, Achromobacter denitrificans, Fusobacterium nucleatum, Streptomyces clavuligenus, Acinetobacter baumanii, Mus musculus, Lachancea kluyveri, Trichomonas vaginalis, Trypanosoma brucei, Pseudomonas stutzeri, Bradyrhizobium japonicum, Mesorhizobium loti, Bos taurus, Nicotiana glutinosa, Vibrio vulnificus, Selenomonas ruminantium, Vibrio parahaemolyticus, Archaeoglobus fulgidus, Haloarcula marismortui, Pyrobaculum aerophilum, Mycobacterium smegmatis MC2 155, Mycobacterium avium subsp. paratuberculosis K-10, Mycobacterium marinum M, Tsukamurella paurometabola DSM 20162, Cyanobium PCC7001, Dictyostelium discoideum AX4, as well as other exemplary species disclosed herein or available as source organisms for corresponding genes.

In certain embodiments, suitable organisms include Acinetobacter baumannii Naval-82, Acinetobacter sp. ADP1, Acinetobacter sp. strain M-1, Actinobacillus succinogenes 130Z, Allochromatium vinosum DSM 180, Amycolatopsis methanolica, Arabidopsis thaliana, Atopobium parvulum DSM 20469, Azotobacter vinelandii DJ, Bacillus alcalophilus ATCC 27647, Bacillus azotoformans LMG 9581, Bacillus coagulans 36D1, Bacillus megaterium, Bacillus methanolicus MGA3, Bacillus methanolicus PB1, Bacillus methanolicus PB-1, Bacillus selenitireducens MLS10, Bacillus smithii, Bacillus subtilis, Burkholderia cenocepacia, Burkholderia cepacia, Burkholderia multivorans, Burkholderia pyrrocinia, Burkholderia stabilis, Burkholderia thailandensis E264, Burkholderiales bacterium Joshi 001, Butyrate producing bacterium L2-50, Campylobacter jejuni, Candida albicans, Candida boidinii, Candida methylica, Carboxydothermus hydrogenoformans, Carboxydothermus hydrogenoformans Z-2901, Caulobacter sp. AP07, Chloroflexus aggregans DSM 9485, Chloroflexus aurantiacus J-10-fl Citrobacter freundii, Citrobacter koseri ATCC BAA-895, Citrobacter youngae, Clostridium, Clostridium acetobutylicum, Clostridium acetobutylicum ATCC 824, Clostridium acidurici, Clostridium aminobutyricum, Clostridium asparagiforme DSM 15981, Clostridium beijerinckii, Clostridium beijerinckii NCIMB 8052, Clostridium bolteae ATCC BAA-613, Clostridium carboxidivorans P7, Clostridium cellulovorans 743B, Clostridium difficile, Clostridium hiranonis DSM 13275, Clostridium hylemonae DSM 15053, Clostridium kluyveri, Clostridium kluyveri DSM 555, Clostridium ljungdahli, Clostridium ljungdahlii DSM 13528, Clostridium methylpentosum DSM 5476, Clostridium pasteurianum, Clostridium pasteurianum DSM 525, Clostridium perfringens, Clostridium perfringens ATCC 13124, Clostridium perfringens str. 13, Clostridium phytofermentans ISDg, Clostridium saccharobutylicum, Clostridium saccharoperbutylacetonicum, Clostridium saccharoperbutylacetonicum N1-4, Clostridium tetani, Corynebacterium glutamicum ATCC 14067, Corynebacterium glutamicum R, Corynebacterium sp. U-96, Corynebacterium variabile, Cupriavidus necator N-1, Cyanobium PCC7001, Desulfatibacillum alkenivorans AK-01, Desulfitobacterium hafniense, Desulfitobacterium metallireducens DSM 15288, Desulfotomaculum reducens MI-1, Desulfovibrio africanus str. Walvis Bay, Desulfovibrio fructosovorans JJ, Desulfovibrio vulgaris str. Hildenborough, Desulfovibrio vulgaris str. Miyazaki F, Dictyostelium discoideum AX4, Escherichia coli, Escherichia coli K-12, Escherichia coli K-12 MG1655, Eubacterium hallii DSM 3353, Flavobacterium frigoris, Fusobacterium nucleatum subsp. polymorphum ATCC 10953, Geobacillus sp. Y4.1MC1, Geobacillus themodenitrificans NG80-2, Geobacter bemidfiensis Bem, Geobacter sulfurreducens, Geobacter sulfurreducens PCA, Geobacillus stearothermophilus DSM 2334, Haemophilus influenzae, Helicobacter pylori, Homo sapiens, Hydrogenobacter thermophilus, Hydrogenobacter thermophilus TK-6, Hyphomicrobium denitrificans ATCC 51888, Hyphomicrobium zavarzinii, Klebsiella pneumoniae, Klebsiella pneumoniae subsp. pneumoniae MGH 78578, Lactobacillus brevis ATCC 367, Leuconostoc mesenteroides, Lysinibacillus fusiformis, Lysinibacillus sphaericus, Mesorhizobiurn loti MAFF303099, Metallosphaera sedula, Methanosarcina acetivorans, Methanosarcina acetivorans C2A, Methanosarcina barkeri, Methanosarcina mazei Tuc01, Methylobacter marinus, Methylobacterium extorquens, Methylobacteriurn extorquens AM1, Methylococcus capsulatas, Methylomonas aminofaciens, Moorella thermoacetica, Mycobacter sp. strain JC1 DSM 3803, Mycobacterium avium subsp. paratuberculosis K-10, Mycobacterium bovis BCG, Mycobacterium gastri, Mycobacterium marinum M, Mycobacterium smegmatis, Mycobacterium smegmatis MC2 155, Mycobacterium tuberculosis, Nitrosopumilus salaria BD31, Nitrososphaera gargensis Ga9. 2, Nocardia farcinica IFM 10152, Nocardia iowensis (sp. NRRL 5646), Nostoc sp. PCC 7120, Ogataea angusta, Ogataea parapolymorpha DL-1 (Hansenula polymorpha DL-1), Paenibacillus peoriae KCTC 3763, Paracoccus denitrificans, Penicillium chrysogenum, Photobacterium profundum 3TCK, Phytofermentans ISDg, Pichia pastoris, Picrophilus torridus DSM9790, Porphyromonas gingivalis, Porphyromonas gingivalis W83, Pseudomonas aeruginosa PA01, Pseudomonas denitrificans, Pseudomonas knackmussii, Pseudomonas putida, Pseudomonas sp, Pseudomonas syringae pv. syringae B728a, Pyrobaculum islandicum DSM 4184, Pyrococcus abyssi, Pyrococcus furiosus, Pyrococcus horikoshii OT3, Ralstonia eutropha, Ralstonia eutropha H16, Rhodobacter capsulatus, Rhodobacter sphaeroides, Rhodobacter sphaeroides ATCC 17025, Rhodopseudomonas palustris, Rhodopseudomonas palustris CGA009, Rhodopseudomonas palustris DX-1, Rhodospirillum rubrum, Rhodospirillum rubrum ATCC 11170, Ruminococcus obeum ATCC 29174, Saccharomyces cerevisiae, Saccharomyces cerevisiae S288c, Salmonella enterica, Salmonella enterica subsp. enterica serovar Typhimurium str. LT2, Salmonella enterica typhimurium, Salmonella typhimurium, Schizosaccharomyces pombe, Sebaldella termitidis ATCC 33386, Shewanella oneidensis MR-1, Sinorhizobium meliloti 1021, Streptomyces coelicolor, Streptomyces griseus subsp. griseus NBRC 13350, Sulfolobus acidocalarius, Sulfolobus solfataricus P-2, Synechocystis str. PCC 6803, Syntrophobacter fumaroxidans, Thauera aromatica, Thermoanaerobacter sp. X514, Thermococcus kodakaraensis, Thermococcus litoralis, Thermoplasma acidophilum, Thermoproteus neutrophilus, Thermotoga maritima, Thiocapsa roseopersicina, Tolumonas auensis DSM 9187, Trichomonas vaginalis G3, Trypanosoma brucei, Tsukamurella paurometabola DSM 20162, Vibrio cholera, Vibrio harveyi ATCC BAA-1116, Xanthobacter autotrophicus Py2, Yersinia intermedia, or Zea mays.

Algae that can be engineered for cannabinoid production include, but are not limited to, unicellular and multicellular algae. Examples of such algae can include a species of rhodophyte, chlorophyte, heterokontophyte (including diatoms), tribophyte, glaucophyte, chlorarachniophyte, euglenoid, haptophyte, cryptomonad, dinoflagellum, phytoplankton, and the like, and combinations thereof. In one embodiment, algae can be of the classes Chlorophyceae and/or Haptophyta.

Microalgae (single-celled algae) produce natural oils that can contain the synthesized cannabinoids. Specific species that are considered for cannabinoid production include, but are not limited to, Neochloris oleoabundans, Scenedesmus dimorphus, Euglena gracilis, Phaeodactylum tricornutum, Pleurochrysis carterae, Prymnesium parvum, Tetraselmis chui, Nannochloropsis gaditiana. Dunaliella salina. Dunaliella tertiolecta, Chlorella vulgaris, Chlorella variabilis, and Chlamydomonas reinhardtii. Additional or alternate algal sources can include one or more microalgae of the Achnanthes, Amphiprora, Amphora, Ankistrodesmus, Asteromonas, Boekelovia, Borodinella, Botryococcus, Bracteococcus, Chaetoceros, Carteria, Chlamydomonas, Chlorococcum, Chlorogonium, Chlorella, Chroomonas, Chrsosphaera, Cricosphaera, Crypthecodinium, Cryptomonas, Cyclotella, Dunaliella, Ellipsoidon, Emiliania. Eremosphaera, Ernodesmius, Euglena, Franceia, Fragilaria, Gloeolhamnion, Haematococcus, Halocafeteria, Hymenomonas, Isochrysis, Lepocinclis, Micractinium, Monoraphidium, Nannochloris, Nannochloropsis, Navicula, Neochloris, Nephrochloris, Nephroselmis, Nitzschia, Ochromonas, Oedogonium, Oocystis, Ostreococcus, Pavlova, Parachlorella, Pascheria, Phaeodactylum, Phagus. Platymonas, Pleurochrsis, Pleurococcus, Prototheca, Pseudochlorella, Pyramimonas, Pvrobotrys, Scenedesmus, Skeletonema, Spyrogyra, Stichococcus, Tetraselmis, Thalassiosira, Viridiella, and Volvox species, and/or one or more cyanobacteria of the Agmenellum, Anabaena, Anabaenopsis, Anacystis, Aphanizomenon, Arthrospira, Asterocapsa, Borzia, Calothrix, Chamaesiphon, Chlorogloeopsis, Chroococcidiopsis, Chroococcus, Crinalium, Cyanobacterium, Cyanobium, Cyanocystis, Cyanospira, Cyanothece, Cylindrospermopsis, Cylindrospermum, Dactylcoccopsis, Dermocarpella, Fischerella, Fremyella, Geitleria, Geitlerinema, Gloeobacter, Gloeocapsa, Gloeothece, Halospirulina, Ivengariella, Leptolyngbya, Limnothrix, Lyngbya, Microcoleus, Microcystis, Mxosarcina, Nodularia, Nostoc, Nostochopsis, Oscillatoria, Phormidium, Planktothrix, Pleurocapsa, Prochlorococcus, Prochloron, Prochlorothrix, Pseudanabaena, Rivularia, Schizothrix, Scvtonema, Spirulina, Stanieria, Starria, Stigonema, Symploca, Synechococcus, Svnechocystis, Tolipothrix, Trichodesmium. Tychonema, and Xenococcus species.

The microalgae host cells can produce a storage oil, which in some embodiments can include hydrocarbons such as triacylglyceride that may be stored in storage bodies of the host cell as well as related products that can include, without limitation, phospholipids, tocopherols, tocotrienols, carotenoids (e.g., alpha-carotene, beta-carotene, lycopene, etc.), xanthophylls (e.g., lutein, zeaxanthin, alpha-cryptoxanthin and beta-crytoxanthin), cannabinoids, isoprenoids and various organic or inorganic compounds. A raw oil may be obtained from the cells by disrupting the cells and isolating the oil. See WO2008/151149, WO2010/06032, WO2011/150410, and WO2011/1504 which disclose heterotrophic cultivation and oil isolation techniques, and all of which are incorporated by reference in their entirety for all purposes. For example, oil may be obtained by cultivating, drying and pressing the cells. The oils produced may also be refined, bleached and deodorized (RBD) to remove phospholipids, free fatty acids and odors as known in the art or as described in WO2010/120939, which is incorporated by reference in its entirety for all purposes. The raw or RBD oils may be used in a variety of food, chemical, pharmaceutical, nutraceutical and industrial products or processes. After recovery of the oil, a valuable residual biomass remains. Uses for the residual biomass can include the production of paper, plastics, absorbents, adsorbents, as animal feed, for human nutrition, or for fertilizer.

The stable carbon isotope value 513C is an expression of the ratio of 13C/12C relative to a standard (e.g. PDB, carbonite of fossil skeleton of Belemnite americana from Peedee formation of South Carolina). The stable carbon isotope value 513C (0/00) of the oils can be related to the 513C value of the feedstock used. The oils can be derived from oleaginous organisms heterotrophically grown, for example, on sugar derived from a C4 plant such as corn or sugarcane. The 513C (0/00) of the oil can be from −10 to −17 0/00 or from −13 to −16 0/00.

The oils disclosed herein can be made by methods using a microalgal host cell. As described above, the microalga can be, without limitation, Chlorophyta, Trebouxiophyceae, Chlorellales, Chlorellaceae, or Chlorophyceae. It has been found that oils from microalgae of Trebouxiophyceae can be distinguished from vegetable oils based on their sterol profiles. Oil produced by Chlorella protothecoides can include sterols such as brassicasterol, ergosterol, campesterol, stigmasterol, and β-sitosterol. Sterols produced by Chlorella can have C24 stereochemistry. Microalgae oils can also include, for example, campesterol, stigmasterol, β-sitosterol, 22,23- dihydrobrassicasterol, proferasterol and clionasterol. Oils produced by the microalgae may be distinguished from plant oils by the presence of sterols with C24 stereochemistry and the absence of C24a stereochemistry in the sterols present. For example, the oils produced may contain 22,23-dihydrobrassicasterol while lacking campesterol; contain clionasterol, while lacking in β-sitosterol, and/or contain poriferasterol while lacking stigmasterol. Alternately, or in addition, the oils may contain significant amounts of Δ7-poriferasterol.

Oleaginous host cells engineered for production of cannabinoids as provided herein can produce an oil with at least 1% of cannabinoid. The oleaginous host cell (e.g., microalgae) can produce an oil, cannabinoid, triglyceride, isoprenoid or derivative of any of these. These host cells can be made by transforming a cell with any of the nucleic acids discussed herein. The transformed cell can be cultivated to produce an oil and, optionally, the oil can be extracted. Oil extracted can be used to produce food, oleochemicals, nutraceuticals, pharmaceuticals or other products.

The oils discussed above alone or in combination can be useful in the production of foods, pharmaceuticals, nutraceuticals, and chemicals. The oils, cannabinoids, isoprenoids, triglycerides can be subjected to decarboxylation, oxidation, light exposure, hydroamino methylation, methoxy-carbonation, ozonolysis, enzymatic transformations, epoxidation, methylation, dimerization, thiolation, metathesis, hydro-alkylation, lactonization, or other chemical processes. After extracting the oil, a residual biomass may be left, which may have use as a fuel, as an animal feed, or as an ingredient in paper, plastic, or other product.

The ability to genetically modify the host is essential for any recombinant production system. The mode of gene transfer technology may be by electroporation, conjugation, transduction or natural transformation.

Genetic Engineering Host Cells

The host cells or microorganisms of the disclosure include host strains or host cells that are genetically engineered to include genetic alterations designed to improve the rate, yield, or titer of cannabinoid production by cell cultures. Various optional genetic manipulations and alterations can be used interchangeably from one host cell to another, depending on the native enzymatic pathways present in the selected host cell.

To genetically modify a parent host cell to produce a genetically modified host cell of the present disclosure, one or more heterologous nucleic acids disclosed herein is introduced stably or transiently into a host cell, using established techniques. Such techniques may include, but are not limited to, electroporation, calcium phosphate precipitation, DEAE-dextran mediated transfection, liposome-mediated transfection, particle bombardment, and the like. For stable transformation, a heterologous nucleic acid will generally further include a selectable marker, e.g., any of several well-known selectable markers such as neomycin resistance, ampicillin resistance, tetracycline resistance, chloramphenicol resistance, kanamycin resistance, hygromycin resistance, G418 resistance, bleomycin resistance, zeocin resistance, and the like. A broad range of plasmids and drug resistance markers are available. The cloning vectors are tailored to the host organisms based on the nature of antibiotic resistance markers that can function in that host.

One or more nucleic acid sequences disclosed herein can be present in an expression vector or construct. Suitable expression vectors may include, but are not limited to, baculovirus vectors, bacteriophage vectors, plasmids, phagemids, cosmids, fosmids, bacterial artificial chromosomes, viral vectors (e.g. viral vectors based on vaccinia virus, poliovirus, adenovirus, adeno-associated virus, SV40, herpes simplex virus, and the like), P1-based artificial chromosomes, yeast plasmids, yeast artificial chromosomes, and any other vectors specific for specific hosts of interest (such as E. coli and yeast). Thus, for example, one or more nucleic acids encoding a cannabinoid pathway gene product is included in any one of a variety of expression vectors for expressing the cannabinoid pathway gene product(s). Such vectors may include chromosomal, non-chromosomal, and synthetic DNA sequences. Numerous additional suitable expression vectors are known to those of skill in the art, and many are commercially available. The following vectors are provided by way of example; for bacterial host cells: pQE vectors (Qiagen), pBluescript plasmids, pNH vectors, lambda-ZAP vectors (Stratagene); pTrc99a, pKK223-3, pDR540, and pRIT2T (Pharmacia); for eukaryotic host cells: pXT1, pSG5 (Stratagene), pSVK3, pBPV, pMSG, and pSVLSV40 (Pharmacia). However, any other plasmid or other vector may be used so long as it is compatible with the host cell.

In some embodiments, a parent host cell is genetically modified to produce a genetically modified host cell of the present disclosure using a CRISPR/Cas9 or other CRISPR system to genetically modify a parent host cell, for example, with one or more heterologous nucleic acids disclosed herein.

In some instances, a chemically synthesized or PCR-amplified nucleic acid fragment, or a nucleic acid fragment excised from a larger nucleic acid molecule or construct, can be introduced into a host cell and optionally integrated into a nucleic acid molecule of the host cell, for example, using CRISPR technology. A nucleic acid fragment introduced into a host cell may or may not include a selectable marker, and may or may not include an expression cassette. For example, a nucleic acid fragment introduced into a host cell for cas9 engineering (or engineering via another RNA-guided endonuclease, e.g., Cpf1) can optionally include the coding sequence of a gene or a portion thereof in the absence of a promoter sequence, or alternatively, may include a promoter sequence or portion thereof in the absence of a complete coding sequence linked to the promoter sequence. Vectors, constructs, and nucleic acid fragments designed for introduction into a host cell can in some embodiments optionally include sequences for mediating homologous recombination into a host chromosome or episome.

Heterologous natural or chemically synthesized genes for enzymes may be introduced on high-level expression plasmid vectors or through genomic integration using methods well known to those skilled in the art. Such methods may involve CRISPR technology. Alternatively, genes that are endogenous to the host organism may be up-regulated by genetic element integration methods known to those skilled in the art.

In some embodiments, one, two, three, four, or more of the nucleic acid sequences disclosed herein that encode an enzyme or other polypeptide that functions in a pathway for producing a cannabinoid or OA or a derivative thereof, or a pathway that reduces byproduct formation, are present in a single expression vector or construct. In some embodiments, two, three, four or more nucleic acid sequences disclosed herein that encode an enzyme or other polypeptide that functions in a pathway for producing a cannabinoid or OA or a derivative thereof, or a pathway that reduces byproduct formation, are present in are in separate expression vectors or constructs. In some embodiments, one, two, three, four, or more nucleic acid sequences that encode an enzyme or other polypeptide that functions in a pathway for producing a cannabinoid or OA or a derivative thereof, or a pathway that reduces byproduct formation, are integrated into a chromosome or episome using an RNA-guided nuclease such as a CRISPR RNA-guided nuclease. Multiple genes encoding enzymes can be inserted into the host chromosome or episome individually, for example, sequentially, or multiple genes may be in inserted into the host chromosome or episome together.

Promoters used for driving transcription of genes in S. cerevisiae and other yeasts are well known in the art and include DNA elements that are regulated by glucose concentration in the growth media, such as the alcohol dehydrogenase-2 (ADH2) promoter. Other regulated promoters or inducible promoters, such as those that drive expression of the GAL1, MET25 and CUP1 genes, are used when conditional expression is required. GAL1 and CUP1 are induced by galactose and copper, respectively, whereas MET25 is induced by the absence of methionine. In some embodiments, one or more of the exogenous polynucleotides is operably linked to a glucose regulated promoter. In some embodiments, expression of one or more of the exogenous polynucleotides is driven by an alcohol dehydrogenase-2 promoter. Other promoters drive strongly transcription in a constitutive manner. Such promoters include, without limitation, the control elements for highly expressed yeast glycolytic enzymes, such as glyceraldehyde-3-phosphate dehydrogenase (GPD), phosphoglycerate kinase (PGK), pyruvate kinase (PYK), triose phosphate isomerase (TPI) and alcohol dehydrogenase-1 (ADH1). Another strong constitutive promoter that may be used is that from the S. cerevisiae transcription elongation factor EF-1 alpha gene (TEF1) (Partow et al., Yeast. 2010, (11):955-64). Promoters for engineering of bacterial hosts are well-known in the art and include inducible promoters such as example, the ara, lac, trc, tet, and cumate-regulated promoters.

In the above embodiments, the nucleic acid sequences may optionally be chemically synthesized genes, with codon optimization for the host being genetically engineered, that encode a wild type or mutant enzyme from another species or the host species.

Engineering of a host cells as provided herein can include expressing a variant of a naturally-occurring enzyme in the host cell. For making variants, mutagenesis methods are well known in the art and include, for example, error-prone PCR (Leung et al. (1989) Technique 1:11-15; and Caldwell et al. (1992) PCR Methods Applic. 2:28-33), oligonucleotide directed mutagenesis (Reidhaar-Olson et al. (1988) Science 241:53-57), assembly PCR (U.S. Pat. No. 5,965,408), and sexual PCR mutagenesis (Stemmer (1994) PNAS, USA 91:10747-10751. Cassette mutagenesis can be used to generate mutant proteins (Richards, J. H. (1986) Nature 323:187; Ecker et al. (1987) J. Biol. Chem. 262:3524-3527); to insert or replace individual codons (Kegler-Ebo et al. (1994) Nucleic Acids Res. 22(9):1593-1599), or to make variants of sequences comprising regulatory sequences (e.g., ribosome binding sites, see, e.g., Barrick et al. (1994) Nucleic Acids Res. 22(7):1287-1295); Wilson et al. (1994) Biotechniques 17:944-953). Recursive ensemble mutagenesis (Arkin et al. (1992) PNAS, USA 89:7811-7815) or exponential ensemble mutagenesis (Delegrave et al. (1993) Biotech. Res. 11:1548-1552) can also be used to generate nucleotide sequence variants. Random and site-directed mutagenesis can also be used (Arnold (1993) Curr. Opin. Biotech. 4:450-455).

Variants of enzymes of interest can also be created by in vivo mutagenesis. In some embodiments, random mutations in a nucleic acid sequence are generated by propagating the polynucleotide sequence in a bacterial strain, such as an E. coli strain, which carries mutations in one or more of the DNA repair pathways. Such “mutator” strains have a higher random mutation rate than that of a wild-type strain. Propagating a DNA sequence in one of these strains will eventually generate random mutations within the DNA. Mutator strains suitable for use for in vivo mutagenesis are described in, for example, PCT International Publication No. WO 91/16427. Standard methods of in vivo mutagenesis can be used. For example, host cells, comprising one or more polynucleotide sequences that include an open reading frame for an ACC polypeptide, as well as operably-linked regulatory sequences, can be subject to mutagenesis via exposure to radiation (e.g., UV light or X-rays) or exposure to chemicals (e.g., ethylating agents, alkylating agents, or nucleic acid analogs). In some host cell types, for example, bacteria, yeast, and plants, transposable elements can also be used for in vivo mutagenesis.

The cannabinoid-producing engineered cells of the invention may be made by transforming a host cell, either through genomic integration or using episomal plasmids (also referred to as expression vectors, or simply vectors) with at least one nucleotide sequence encoding enzymes involved in the engineered metabolic pathways. As used herein the term “nucleotide sequence” and “nucleic acid sequence” are used interchangeably and mean a polymer of RNA or DNA, single- or double-stranded, optionally containing synthetic, non-natural or altered nucleotide bases. A nucleotide sequence may comprise one or more segments of cDNA, genomic DNA, synthetic DNA, or RNA. In some embodiments, the nucleotide sequence is codon-optimized to reflect the typical codon usage of the host cell without altering the polypeptide encoded by the nucleotide sequence. In certain embodiments, the term “codon optimization” or “codon-optimized” refers to modifying the codon content of a nucleic acid sequence without modifying the sequence of the polypeptide encoded by the nucleic acid to optimize expression in a particular host cell. For example, genes for any of the polypeptides described with reference to the SEQ ID NOs described herein can be codon optimed for expression in a desired host cell.

Methods of introducing exogenous nucleic acids into plant cells are also well known in the art. Such plant cells are considered “transformed.” Suitable methods may include viral infection (such as double stranded DNA viruses), transfection, conjugation, protoplast fusion, electroporation, particle gun technology, calcium phosphate precipitation, direct microinjection, silicon carbide whiskers technology, Agrobacterium-mediated transformation, CRISPR/Cas9-mediated genome editing, and the like. The choice of method is generally dependent on the type of cell being transformed and the circumstances under which the transformation is taking place (e.g., in vitro, ex vivo, or in vivo).

In other aspects, engineering may be employed to reduce the production of byproducts, e.g., ethanol that utilize carbon sources that lead to reduced utilization of that carbon source for cannabinoid production. Such genes may be completely “knocked out” of the genome by deletion, or may be reduced in activity through reduction of promoter strength or the like. Such genes include those for the enzymes alcohol dehydrogenase and lactate dehydrogenase, for example.

Given the teachings and guidance provided herein, those skilled in the art will understand that enzymatic activity or expression can be attenuated using well known methods. Reduction of the activity or amount of an enzyme can mimic complete disruption of a gene if the reduction causes activity of the enzyme to fall below a critical level that is normally required for a pathway to function. Reduction of enzymatic activity by various techniques rather than use of a gene disruption can be important for an organism's viability. Methods of reducing enzymatic activity that result in similar or identical effects of a gene disruption include, but are not limited to: reducing gene transcription or translation; destabilizing mRNA, protein or catalytic RNA; and mutating a gene that affects enzyme activity or kinetics (see Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, Md. (1999). Natural or imposed regulatory controls can also accomplish enzyme attenuation including: promoter replacement (see Wang et al., Mol. Biotechnol. 52(2):300-308 (2012)); loss or alteration of transcription factors (Dietrick et al., Annu. Rev. Biochem. 79:563-590 (2010); and Simicevic et al., Mol. Biosyst. 6(3):462-468 (2010)); introduction of inhibitory RNAs or peptides such as siRNA, antisense RNA, RNA or peptide/small-molecule binding aptamers, ribozymes, aptazymes and riboswitches (Wieland et al., Methods 56(3):351-357 (2012); O'Sullivan, Anal. Bioanal. Chem. 372(1):44-48 (2002); and Lee et al., Curr. Opin. Biotechnol. 14(5):505-511 (2003)); and addition of drugs or other chemicals that reduce or disrupt enzymatic activity such as an enzyme inhibitor, an antibiotic or a target-specific drug.

One skilled in the art will also understand and recognize that attenuation of an enzyme can be done at various levels. For example, at the gene level, a mutation causing a partial or complete null phenotype, such as a gene disruption or a mutation causing epistatic genetic effects that mask the activity of a gene product (Miko, Nature Education 1(1) (2008)), can be used to attenuate an enzyme. At the gene expression level, methods for attenuation include: coupling transcription to an endogenous or exogenous inducer such as isopropylthio-β-galactoside (IPTG), then adding low amounts of inducer or no inducer during the production phase (Donovan et al., J. Ind. Microbiol. 16(3):145-154 (1996); and Hansen et al., Curr. Microbiol. 36(6):341-347 (1998)); introducing or modifying a positive or a negative regulator of a gene; modify histone acetylation/deacetylation in region in a eukaryotic chromosomal region where a gene is integrated (Yang et al., Curr. Opin. Genet. Dev. 13(2):143-153 (2003) and Kurdistani et al., Nat. Rev. Mol. Cell Biol. 4(4):276-284 (2003)); introducing a transposition to disrupt a promoter or a regulatory gene (Bleykasten-Brosshans et al., C. R. Biol. 33(8-9):679-686 (2011); and McCue et al., PLoS Genet. 8(2):e1002474 (2012)); flipping the orientation of a transposable element or promoter region so as to modulate gene expression of an adjacent gene (Wang et al., Genetics 120(4):875-885 (1988); Hayes, Annu. Rev. Genet. 37:3-29 (2003); in a diploid organism, deleting one allele resulting in loss of heterozygosity (Daigaku et al., Mutation Research/Fundamental and Molecular Mechanisms of Mutagenesis 600(1-2)177-183 (2006)); introducing nucleic acids that increase RNA degradation (Houseley et al., Cell, 136(4):763-776 (2009); or in bacteria, for example, introduction of a transfer-messenger RNA (tmRNA) tag, which can lead to RNA degradation and ribosomal stalling (Sunohara et al., RNA 10(3):378-386 (2004); and Sunohara et al., J. Biol. Chem. 279:15368-15375 (2004)). At the translational level, attenuation can include: introducing rare codons to limit translation (Angov, Biotechnol. J. 6(6):650-659 (2011)); introducing RNA interference molecules that block translation (Castel et al., Nat. Rev. Genet. 14(2):100-112 (2013); and Kawasaki et al., Curr. Opin. Mol. Ther. 7(2):125-131 (2005); modifying regions outside the coding sequence, such as introducing secondary structure into an untranslated region (UTR) to block translation or reduce efficiency of translation (Ringnér et al., PLoS Comput. Biol. 1(7):e72 (2005)); adding RNAase sites for rapid transcript degradation (Pasquinelli, Nat. Rev. Genet. 13(4):271-282 (2012); and Arraiano et al., FEMS Microbiol. Rev. 34(5):883-932 (2010); introducing antisense RNA oligomers or antisense transcripts (Nashizawa et al., Front. Biosci. 17:938-958 (2012)); introducing RNA or peptide aptamers, ribozymes, aptazymes, riboswitches (Wieland et al., Methods 56(3):351-357 (2012); O'Sullivan, Anal. Bioanal. Chem. 372(1):44-48 (2002); and Lee et al., Curr. Opin. Biotechnol. 14(5):505-511 (2003)); or introducing translational regulatory elements involving RNA structure that can prevent or reduce translation that can be controlled by the presence or absence of small molecules (Araujo et al., Comparative and Functional Genomics, Article ID 475731, 8 pages (2012). At the level of enzyme localization and/or longevity, enzyme attenuation can include: adding a degradation tag for faster protein turnover (Hochstrasser, Annual Rev. Genet. 30:405-439 (1996); and Yuan et al., PLoS One 8(4):e62529 (2013)); or adding a localization tag that results in the enzyme being secreted or localized to a subcellular compartment in a eukaryotic cell, where the enzyme would not be able to react with its normal substrate Nakai et al. Genomics 14(4):897-911 (1992); and Russell et al., J. Bact. 189(21)7581-7585 (2007)). At the level of post-translational regulation, enzyme attenuation can include: increasing intracellular concentration of known inhibitors; or modifying post-translational modified sites (Mann et al., Nature Biotech. 21:255-261 (2003)). At the level of enzyme activity, enzyme attenuation can include: adding an endogenous or an exogenous inhibitor, such as an enzyme inhibitor, an antibiotic, or a target-specific drug, to reduce enzyme activity; limiting availability of essential cofactors, such as vitamin B12, for an enzyme that requires the cofactor; chelating a metal ion that is required for enzyme activity; or introducing a dominant negative mutation. The applicability of a technique for attenuation described above can depend upon whether a given host microbial organism is prokaryotic or eukaryotic, and it is understood that a determination of what is the appropriate technique for a given host can be readily made by one skilled in the art.

A CRISPR/Cas9 (or other RNA-guided endonuclease) system can be used to generate a transgenic (genetically modified) microorganism or plant cell of the present disclosure, including generating regulatory mutants (e.g., “knockdown” or decreased expression of endogenous genes) and knockout mutations. CRISPR/Cas9 and other CRISPR systems and methods for mutating promoters, causing insertions in the upstream regions of genes that negatively affect gene expression, and disrupting genes are also known in the art. See, e.g., Bortesi and Fischer (2015) Biotechnol. Advances 33:41; Fan et al. (2015) Sci. Reports 5:12217; Ajjawi et al. (2017) Nature Biotech 35:647-652.

Fermentation

In yet another aspect, methods for producing a cannabinoid, or precursor as described herein, that include incubating a culture of an engineered host cell as provided herein to produce the cannabinoid or precursor. The methods can further include recovering the cannabinoid from the cells, the culture medium, or whole culture.

The cultures comprise cells engineered for the production of cannabinoids in a culture medium. In various embodiments the engineered host cells can be bacterial, fungal, or algal cells, including cyanobacterial and eukaryotic microalgal cells. In embodiments where the cells are heterotrophic cells, the culture medium includes at least one carbon source that is also an energy source. The culture medium can include one, two, three, or more carbon sources that are not primary energy sources. Nonlimiting examples of feed molecules that can be included in the culture medium include acetate, malonate, oxaloacetate, aspartate, glutamate, beta-alanine, alpha-alanine, hexanoate, hexanol, prenol, isoprenol, and geraniol. Further examples of compounds that can be provided in the culture medium include, without limitation, biotin, thiamine, pantotheine, and 4-phosphopantetheine.

In some embodiments, acetate is provided in the culture medium. In some embodiment, acetate and hexanoate are provided in the culture medium. In some embodiments, malonate and hexanoate are provided in the culture medium. In either of these embodiments, the culture medium can further include prenol, isoprenol, or geraniol. In some embodiments, aspartate, hexanoate, and prenol, isoprenol, or geraniol are present in the culture medium.

Depending on the desired microorganism or strain to be used, the appropriate culture medium may be used. For example, descriptions of various culture media may be found in “Manual of Methods for General Bacteriology” of the American Society for Bacteriology (Washington D.C., USA, 1981). As used here, culture medium, or simply “medium” as it relates to the growth source refers to the starting medium be it in a solid or liquid form. “Cultured medium”, on the other hand and as used here refers to medium (e.g. liquid medium) containing microbes that have been fermentatively grown and can include other cellular biomass. The medium generally includes one or more carbon sources, nitrogen sources, inorganic salts, vitamins and/or trace elements. “Whole culture” as used herein refers to cultured cells plus the culture medium they are cultured in.

Exemplary carbon sources include sugar carbons such as sucrose, glucose, galactose, fructose, mannose, isomaltose, xylose, maltose, arabinose, cellobiose and 3-, 4-, or 5-oligomers thereof. Other carbon sources include carbon sources such as methanol, ethanol, glycerol, formate and fatty acids. Still other carbon sources include carbon sources from gas such as synthesis gas, waste gas, methane, CO, CO2 and any mixture of CO, CO2 with H2. Other carbon sources can include renewal feedstocks and biomass. Exemplary renewal feedstocks include cellulosic biomass, hemicellulosic biomass and lignin feedstocks.

In some embodiments, culture conditions include aerobic, microaerobic, anaerobic or substantially anaerobic growth or maintenance conditions. Exemplary aerobic, microaerobic, and anaerobic conditions have been described previously and are well known in the art. Exemplary anaerobic conditions for fermentation processes are disclosed, for example, in U.S. Patent Application Publication No 2009/0047719, filed Aug. 10, 2007. Any of these conditions can be employed with the microbial organisms as well as other anaerobic conditions well known in the art.

The culture conditions can include, for example, liquid culture procedures as well as fermentation and other large scale culture procedures. Useful yields of the products can be obtained under aerobic, microaerobic, anaerobic or substantially anaerobic culture conditions.

Algae can be cultured photoautotrophically, in the light, without a reduced carbon source that can be used for energy, mixotrophically, where the algae are exposed to light that allows photosynthesis and also use a reduced carbon source provided in the culture medium, or heterotrophically, in the dark, where the cells rely entirely on a reduced carbon source provided in the culture medium for growth and energy.

An exemplary growth condition for achieving, one or more cannabinoid product(s) includes aerobic, microaerobic, anaerobic culture or fermentation conditions. In certain embodiments, the microbial organism can be sustained, cultured or fermented under aerobic, microaerobic, anaerobic or substantially anaerobic conditions. Briefly, anaerobic conditions refer to an environment devoid of oxygen. Conditions include, for example, a culture, batch fermentation or continuous fermentation such that the dissolved oxygen concentration in the medium remains between 0 and 10% of saturation, or higher. Substantially anaerobic conditions also include growing or resting cells in liquid medium or on solid agar inside a sealed chamber maintained with an atmosphere of less than 1% oxygen. The percent of oxygen can be maintained by, for example, sparging the culture with an N2/CO2 mixture or other suitable non-oxygen gas or gases.

The culture conditions can be scaled up and grown continuously for manufacturing cannabinoid product. Exemplary growth procedures include, for example, fed-batch fermentation and batch separation; fed-batch fermentation and continuous separation, or continuous fermentation and continuous separation. All of these processes are well known in the art. Fermentation procedures are particularly useful for the biosynthetic production of commercial quantities of cannabinoid product. Generally, and as with non-continuous culture procedures, the continuous and/or near-continuous production of cannabinoid product will include culturing a cannabinoid producing organism on sufficient nutrients and medium to sustain and/or nearly sustain growth in an exponential phase. Continuous culture under such conditions can include, for example, 1 day, 2, 3, 4, 5, 6 or 7 days or more. Additionally, continuous culture can include 1 week, 2, 3, 4 or 5 or more weeks and up to several months. Alternatively, the desired microorganism can be cultured for hours, if suitable for a particular application. It is to be understood that the continuous and/or near-continuous culture conditions also can include all time intervals in between these exemplary periods. It is further understood that the time of culturing the microbial organism is for a sufficient period of time to produce a sufficient amount of product for a desired purpose.

Fermentation procedures are well known in the art. Briefly, fermentation for the biosynthetic production of cannabinoid product can be utilized in, for example, fed-batch fermentation and batch separation; fed-batch fermentation and continuous separation, or continuous fermentation and continuous separation. Examples of batch and continuous fermentation procedures are well known in the art. Typically cells are grown at a temperature in the range of about 25° C. to about 40° C. in an appropriate medium, as well as up to 70° C. for thermophilic microorganisms.

The culture medium may include a feed molecule that is converted into a cannabinoid precursor, such as, but not limited to, CO₂, acetate, malonate, beta-alanine, aspartate, glutamate, oxaloacetate, hexanoate, hexanol, prenol, isoprenol, or geraniol. The feed molecule can also serve as the main or a supplemental carbon source for cell growth and energy, or can be provided in addition to a sugar, sugar alcohol, polyol, or organic acid that is provided for growth and energy. Additional supplements can optionally include biotin, thiamine, pantothenate, and/or 4′-phosphopantotheine.

The culture medium at the start of fermentation may have a pH of about 4 to about 7. The pH may be less than 11, less than 10, less than 9, or less than 8. In other embodiments the pH may be at least 2, at least 3, at least 4, at least 5, at least 6, or at least 7. In other embodiments, the pH of the medium may be about 6 to about 9.5; 6 to about 9, about 6 to 8 or about 8 to 9.

Exemplary fermentation processes include, but are not limited to, fed-batch fermentation and batch separation; fed-batch fermentation and continuous separation; and continuous fermentation and continuous separation. In an exemplary batch fermentation protocol, the production organism is grown in a suitably sized bioreactor sparged with an appropriate gas. Under anaerobic conditions, the culture is sparged with an inert gas or combination of gases, for example, nitrogen, N₂/CO₂ mixture, argon, helium, and the like. As the cells grow and utilize the carbon source, additional carbon source(s) and/or other nutrients are fed into the bioreactor at a rate approximately balancing consumption of the carbon source and/or nutrients. The temperature of the bioreactor is maintained at a desired temperature, generally in the range of 22-37 degrees C., but the temperature can be maintained at a higher or lower temperature depending on the growth characteristics of the production organism and/or desired conditions for the fermentation process. Growth continues for a desired period of time to achieve desired characteristics of the culture in the fermenter, for example, cell density, product concentration, and the like. In a batch fermentation process, the time period for the fermentation is generally in the range of several hours to several days, for example, 8 to 24 hours, or 1, 2, 3, 4 or 5 days, or up to a week, depending on the desired culture conditions. The pH can be controlled or not, as desired, in which case a culture in which pH is not controlled will typically decrease to pH 3-6 by the end of the run. Upon completion of the cultivation period, the fermenter contents can be passed through a cell separation unit, for example, a centrifuge, filtration unit, and the like, to remove cells and cell debris. In the case where the desired product is expressed intracellularly, the cells can be lysed or disrupted enzymatically or chemically prior to or after separation of cells from the fermentation broth, as desired, in order to release additional product. The fermentation broth can be transferred to a product separations unit. Isolation of product occurs by standard separations procedures employed in the art to separate a desired product from dilute aqueous solutions. Such methods include, but are not limited to, liquid-liquid extraction using a water immiscible organic solvent (e.g., toluene or other suitable solvents, including but not limited to diethyl ether, ethyl acetate, tetrahydrofuran (THF), methylene chloride, chloroform, benzene, pentane, hexane, heptane, petroleum ether, methyl tertiary butyl ether (MTBE), dioxane, and the like) to provide an organic solution of the product, if appropriate, standard distillation methods, and the like, depending on the chemical characteristics of the product of the fermentation process.

In an exemplary fully continuous fermentation protocol, the production organism is generally first grown up in batch mode in order to achieve a desired cell density. When the carbon source and/or other nutrients are exhausted, feed medium of the same composition is supplied continuously at a desired rate, and fermentation liquid is withdrawn at the same rate. Under such conditions, the product concentration in the bioreactor generally remains constant, as well as the cell density. The temperature of the fermenter is maintained at a desired temperature, as discussed above. During the continuous fermentation phase, it is generally desirable to maintain a suitable pH range for optimized production. The pH can be monitored and maintained using routine methods, including the addition of suitable acids or bases to maintain a desired pH range. The bioreactor is operated continuously for extended periods of time, generally at least one week to several weeks and up to one month, or longer, as appropriate and desired. The fermentation liquid and/or culture is monitored periodically, including sampling up to every day, as desired, to assure consistency of product concentration and/or cell density. In continuous mode, fermenter contents are constantly removed as new feed medium is supplied. The exit stream, containing cells, medium, and product, are generally subjected to a continuous product separations procedure, with or without removing cells and cell debris, as desired. Continuous separations methods employed in the art can be used to separate the product from dilute aqueous solutions, including but not limited to continuous liquid-liquid extraction using a water immiscible organic solvent (e.g., toluene or other suitable solvents, including but not limited to diethyl ether, ethyl acetate, tetrahydrofuran (THF), methylene chloride, chloroform, benzene, pentane, hexane, heptane, petroleum ether, methyl tertiary butyl ether (MTBE), dioxane, and the like), standard continuous distillation methods, and the like, or other methods well known in the art.

Suitable purification and/or assays to test, e.g., a cannabinoid can be performed using well known methods. For example, product and byproduct formation in the engineered production host can be monitored. The final product and intermediates, and other organic compounds, can be analyzed by methods such as HPLC (High Performance Liquid Chromatography), GC-MS (Gas Chromatography-Mass Spectroscopy) and LC-MS (Liquid Chromatography-Mass Spectroscopy) or other suitable analytical methods using routine procedures well known in the art. The release of product in the fermentation broth can also be tested with the culture supernatant. Byproducts and residual glucose can be quantified by HPLC using, for example, a refractive index detector for glucose and alcohols, and a UV detector for organic acids (Lin et al., Biotechnol. Bioeng. 90:775-779 (2005)), or other suitable assay and detection methods well known in the art. The individual enzyme or protein activities from the exogenous DNA sequences can also be assayed using methods well known in the art.

Cannabinoids can be separated from other components in the culture using a variety of methods well known in the art. Such separation methods include, for example, extraction procedures as well as methods that include liquid-liquid extraction, pervaporation, evaporation, filtration, membrane filtration (including reverse osmosis, nanofiltration, ultrafiltration, and microfiltration), membrane filtration with diafiltration, membrane separation, reverse osmosis, electrodialysis, distillation, extractive distillation, reactive distillation, azeotropic distillation, crystallization and recrystallization, centrifugation, extractive filtration, ion exchange chromatography, size exclusion chromatography, adsorption chromatography, carbon adsorption, hydrogenation, and ultrafiltration. For example, the amount of cannabinoid or other product(s), including a polyketide, produced in a bio-production media generally can be determined using any of methods such as, for example, high performance liquid chromatography (HPLC), gas chromatography (GC), GC/Mass Spectroscopy (MS), or spectrometry. All of the above methods are well known in the art.

The disclosure also provides compositions that are enriched for desired cannabinoids, analogs, and derivatives thereof, for example, CBGA, THCV, THCVA, CBDV, CBDVA, CBN, CBNA, CBD, CBDA, CBC, CBCA, CBGV, CBGVA, CBG, CBCV, CBCVA, THC, THCA, analogs, or derivatives thereof, or combinations thereof are disclosed herein. Such enriched compositions include those that are pharmaceutical compositions as well as those that are used for non-pharmaceutical purposes, including medicinal purposes. Accordingly, in some embodiments, provided are compositions, such as pharmaceutical compositions or medicinal compositions, with CBGA and/or CBG that are 90% or greater, 91% or greater, 92% or greater, 93% or greater, 94% or greater, 95% or greater, 96% or greater, 97% or greater, 98% or greater, 99% or greater, 99.2% or greater, 99.4% or greater, 99.5% or greater, 99.6% or greater, 99.7% or greater, 99.8% or greater, 99.9% or greater, 99.95% or greater or even 100% CBGA or its decarboxylated derivative CBG, and cannabinoid compounds.

In some embodiments, culture conditions include anaerobic or substantially anaerobic growth or maintenance conditions. Exemplary anaerobic conditions have been described previously and are well known in the art. Exemplary anaerobic conditions for fermentation processes are described herein and are described, for example, in U.S. publication 2009/0047719, filed Aug. 10, 2007. Any of these conditions can be employed with the non-naturally occurring microbial organisms as well as other anaerobic conditions well known in the art.

Purification and Analysis

Suitable purification and/or assays to test, e.g., for the production of any cannabinoid (e.g., CBGA) or metabolic intermediate or precursor can be performed using well known methods. Suitable replicates such as triplicate cultures can be grown for each engineered strain to be tested. For example, product and byproduct formation in the engineered production host can be monitored. The final product and intermediates, and other organic compounds, can be analyzed by methods such as HPLC (High Performance Liquid Chromatography), GC-MS (Gas Chromatography-Mass Spectroscopy) and LC-MS (Liquid Chromatography-Mass Spectroscopy) or other suitable analytical methods using routine procedures well known in the art. The release of product in the fermentation broth can also be tested with the culture supernatant. Byproducts and residual glucose can be quantified by HPLC using, for example, a refractive index detector for glucose and alcohols, and a UV detector for organic acids (Lin et al., Biotechnol. Bioeng. 90:775-779 (2005)), or other suitable assay and detection methods well known in the art. The individual enzyme or protein activities from the exogenous DNA sequences can also be assayed using methods well known in the art.

CBGA or other target molecules may be separated from other components in the culture using a variety of methods well known in the art. Such separation methods include, for example, extraction procedures as well as methods that include continuous liquid-liquid extraction, pervaporation, evaporation, filtration, membrane filtration (including reverse osmosis, nanofiltration, ultrafiltration, and microfiltration), membrane filtration with diafiltration, membrane separation, reverse osmosis, electrodialysis, distillation, extractive distillation, reactive distillation, azeotropic distillation, crystallization and recrystallization, centrifugation, extractive filtration, ion exchange chromatography, size exclusion chromatography, adsorption chromatography, carbon adsorption, hydrogenation, and ultrafiltration. All of the above methods are well known in the art.

### Example 1—Plasmid Construction, Strain Modification, Production of Olivetolic Acid and CBGA in E. coli, and Analytical Methods for Detecting Cannabinoids

Plasmid Construction

Pathway gene plasmid. pZ vector (Novagen) was used to clone acetyl-CoA carboxylase genes, prenyltransferase genes, biotin ligases, olivetol synthase, olivetolic acid cyclase, genes under control of a constitutive or inducible promoter. Gene fragments were directly synthesized and assembled with the vector backbone with Golden Gate Assembly (New England Biolabs, MA, USA) or Gibson Assembly® (New England Biolabs, MA, USA).

pRed_Cas9 plasmid. The Cas9 gene from Streptococcus pyogenes, lambda red components from bacteriophage lambda, pSC101 temperature sensitive origin of replication, arabinose operon, and β-lactamase gene were assembled into a single plasmid with golden gate assembly method. Lambda red components were driven by an arabinose-inducible promoter pBAD and Cas9 gene was under control of a rhamnose-inducible promoter.

pGuide plasmid. A pair of 20 nt oligos were designed to target genomic locus for editing. Complementary oligos with overhangs were ordered from IDT and annealed to generate N20 part. gRNA without its N20 (5′-GTTTTAGAGCTAGAAATAGCAAGT TAAAATAAGGCTAGTCCGTTATCAACTTGAAAAAGTGGCACCGAGTCGGTGCTT-3′) (SEQ ID NO: 201) and its promoter were directly synthesized and assembled with a sacBK cassette into a pZE vector to generate a base plasmid for cloning N20 part. The base plasmid was digested with restriction enzyme BsaI and assembled with N20 with a Golden Gate Assembly reaction. DNA editing templates containing a homology arm of 50-500 bp to the target genome locus were PCR-amplified with KOD polymerase (NEB) or directly synthesized.

Strain Modification

Gene deletions and insertions were carried out using CRISPR editing. Escherichia coli strain was grown in 30 mL LB at 30° C. to an OD600 of 0.6 and then made electrocompetent by concentrating 100-fold and washing three times with ice-cold water or glycerol. 10-50 ng of pRed_Cas9 plasmid was used for electroporation, shocked cells were added to 0.5 mL LB, incubated 1 h at 30° C., and then plated on LB petri dishes carrying carbenicillin antibiotics. A single colony was inoculated to a 15 ml falcon tube in LB+100 mg/L carbenicillin for an overnight cultivation at 30° C., rpm 225. The seed culture was then inoculated to a 250 ml flask in 30 ml LB+2% (w/v) arabinose+0.2% (w/v) rhamnose+carb to an OD₆₀₀ of 0.6 to make electrocompenent cells. 50-100 ng of guide plasmid and 100-500 ng editing templates were used for electroporation. The resulted strains were grown in LB+carb+0.2% rhamnose for 1-4 h. The culture was plated on LB+100 mg/L carbenicillin+50 mg/L kanamycin+0.2% rhamnose agar plates, and the colonies were analyzed by colony PCR with a forward primer upstream of the left homology arm and a reverse primer downstream of the right homology arm. Colonies with expected PCR product were subjected to verification by DNA sequencing for further confirmation. At last, the temperature sensitive pRed_Cas9 plasmid and sacBK-containing guide plasmid were cured by growing the edited strains on LB+10% sucrose liquid medium or agar plates 37° C. overnight.

OLA Production in Escherichia coli

To test OLA production in Escherichia coli, strains comprising olivetol synthase, olivetolic acid cyclase genes were inoculated to multi-well plates or flasks containing LB supplemented with 1% glycerol and appropriate concentrations of antibiotics. After 16 hours of cultivation at 30°, the cells were transferred to fresh medium with a starting OD of 1.2 and cultivated for 5 h at 30° C. to reach an OD of 2.0-5.0. The cultures were then spun down and resuspended in minimal medium supplemented with 4% glycerol, 2% casAA, 100 μM biotin, and appropriate concentrations of antibiotics with a starting OD600 of 0.05. After 19 hours of cultivation at 30° C., the seed culture was spun down and resuspended in minimal medium supplemented with 4% glycerol, 2% casAA, 100 uM biotin, 1 mg/L thiamine, and 4 mM hexanoic acid to reach a starting OD600 of 0.5-5.0. The resulted cultures were inoculated to a multi-well plate to grow for 24 h at 30° C., 600 rpm. At the end of the cultivation, 20 μl of each culture was diluted in 180 μl culture medium to measure its optical density using a 96-well transparent flat-bottomed microplate. The remaining cell cultures were centrifuged for 20 minutes at 4,000×g, and 100 μL of each supernatant was transferred to a 96-well plate for analytical quantification of OLA, OL, PDAL, and hexanoic acid.

CBGA Production in Escherichia coli

To test CBGA production in Escherichia coli strains comprising olivetol synthase, olivetolic acid cyclase, prenyltransferase genes, genes encoding non-MVA, non-MEP pathway enzymes, were inoculated to multi-well plates or flasks containing LB supplemented with 1% glycerol and appropriate concentrations of antibiotics. After 16 hours of cultivation at 30° C., the cells were transferred to fresh medium with a starting OD of 1.2 and cultivated for 5 h at 30° C. to reach an OD of 2.0-5.0. The cultures were then spun down and resuspended in minimal medium supplemented with 4% glycerol, 2% casAA, and appropriate concentrations of antibiotics with a starting OD of 0.05. After 19 hours of cultivation at 30° C., the seed cultures were spun down and resuspended in minimal medium supplemented with 4% glycerol, 2% casAA, 20 mM prenol or isoprenol, 4 mM hexanoic acid or 400 uM OLA to obtain a starting OD of 0.5-5.0. The resulted cultures were inoculated to a multi-well plate to grow for 24-48 h at 30° C., 600 rpm. In some examples, 20 mM prenol or isoprenol was spiked in during the cultivation. At the end of the cultivation, 20 μl of each culture was diluted in 180 ul culture medium to measure its optical density using a 96-well transparent flat-bottomed microplate. 100 ul of the remaining cell cultures were treated with 900 μl acetonitrile and centrifuged. Supernatant was transferred to a multi-well plate for subsequent LCMS analysis of CBGA, OLA, OL, PDAL, prenol or isoprenol, and hexanoic acid.

Analytical Analysis of Cannabinoid Intermediates

Olivetol, PDAL, OLA, HTAL, CBGA and combinations thereof may be analyzed by LCMS or LCMS/MS methods using C18 reversed phase chromatography coupled to either Exactive (Thermofisher) or QTrap 4500 (Sciex) mass spectrometers.

In vitro enzymatic reactions, whether conducted in cell lysate or using purified proteins, can be first treated with 6 volumes of organic solvent (acetonitrile containing internal standards) to precipitate proteins, the supernatant can be recovered and further diluted for LCMS analysis, if necessary. For in vivo samples, cell cultures are spun down and supernatant can be directly used for LCMS analysis to quantify OLA, OL, and PDAL. For CBGA analysis, cell cultures are first treated with acetonitrile to solubilize CBGA.

Reversed phase LCMS may be used, and compounds can be identified by their LC retention times and MRM transitions specific to the compounds. LCMSMS analysis can be conducted on Shimadzu UHPLC system coupled with AB Sciex QTRAP4500 mass spectrometer. Agilent Eclipse XDB C18 column (4.6×3.0 mm, 1.8 um) may be used with a 1-min gradient elution at 1 mL/min using water containing 0.1% ammonia acetate as mobile phase A and 90% methanol containing 0.1% ammonia acetate as mobile phase B. The LC column temperature can be maintained at 45° C. Negative ionization mode can be used for all the analytes.

### Example 2—OLA Production Using Heterologous ACC Genes

ACC catalyzes the conversion of acetyl-CoA into Mal-CoA which is believed to be a rate-limiting reagent in the production of OLA and other downstream Mal-CoA products including CBGA

In order to investigate and compare the catalytic potential of ACC from a variety of species, including multi-subunit ACC proteins and multidomain acc proteins, heterologous acc genes were introduced into E. coli K-12 MG1655 strain comprising olivetol synthase, olivetolic acid cyclase, hexanoyl-CoA ligase genes as described in Example 1.

Acc A, B, C, D genes from Nocardia farcinica, Chloroflexus aurantiacus, and Corynebacterium glutamicum were cloned into pZ vector as described in Example 1. Similarly, multidomain Acc gene from Arabidopsis thaliana and Mucor circinelloides were cloned into pZ vectors as described in Example 1. E. coli strains comprising hexanoyl-CoA ligase, olivetol synthase, olivetolic acid cyclase genes were transformed with the pZ vectors comprising the Acc genes. The results are provided in Table 4 and demonstrate that certain heterologous ACC genes result in significantly greater OLA production when expressed in E. coli.

TABLE 4
OLA Production From Heterologous ACC Proteins In E. coli.

| | OLA Concentration |
|---|---|
| ACC Source | (μM) |
| E. coli K-12 MG1655 (bacterium) | 0.9 |
| Nocardia farcinica (bacterium) | 0.9 |
| Arabidopsis thaliana (plant) | 1.1 |
| Chloroflexus aurantiacus (bacterium) | 1.2 |
| Saccharomyces cerevisiae (fungus) | 1.2 |
| Corynebacterium glutamicum | 42.0 |
| (bacterium) | |
| Mucor circinelloides (fungus) | 132.0 |

### Example 3—Integration of Mucor circinelloides Gene into E. coli Genome

Mucor circinelloides multidomain acc gene was integrated into the hybC locus of the E. coli genome using CRISPR as described in Example 1. The Mucor circinelloides Acc gene was integrated into the E. coli genome using ACAATCATCCATGCATCGCG (SEQ ID NO: 202) and GTACACGTAGTGGATGCTGA (SEQ ID NO: 203) guide sequences.

### Example 4—FabF Deletion Increases OLA Production

Fatty acid biosynthetic (Fab) pathways consume a significant amount of intracellular Mal-CoA. The present experiment evaluates the effect of Fab blockade on OLA production. The FabF gene was deleted in two different E. coli strains comprising hexanoyl-CoA ligase, olivetol synthase, olivetolic acid cyclase genes: E. coli Kh-13560 and E. coli Kh-13526 using CRISPR as described in Example 1. The guide sequences used for fabF deletion were CCGCAATGATAACCCGCAAG (SEQ ID NO: 204) and CCGCTTGCGGGTTATCATTG (SEQ ID NO: 205).

The results are provided in Table 5 and demonstrate that OLA production is significantly increased in E. coli engineered to delete the FabF gene. E. coli Kh13526 had a higher basal level of OLA production (139.0 μM) versus the E. coli Kh13560 (24.7 μM). However, FabF gene deletion caused an increase in OLA production of about 2-3-fold.

TABLE 5
OLA Production In E. coli Having A FabF
Gene Deletion

| | OLA Concentration |
|---|---|
| ACC Source | (μM) |
| E. coli Kh-13560 | 24.7 |
| E. coli Kh-13560 ΔFabF | 58.3 |
| E. coli Kh-13526 | 139.0 |
| E. coli Kh-13526 ΔFabF | 320.0 |

### Example 5—Integration of Various Genes into E. coli Genome to Increase the Yield of Cannabinoid Production

Prenyltransferase, acc, olivetolic acid cyclase, olivetol synthase, thiM, ipk, idsA, idi, and fadD genes were integrated into E. coli genome using CRISPR as described in Example 1. Briefly, prenyltransferase gene was integrated into E. coli fadE locus, Mucor circinelloides multidomain acc gene was integrated into E. coli hybC locus, OAC gene into fhuA, fadB, and/or fadR loci, thiM was integrated into E. coli poxB locus, ipk was integrated into E. coli thiM locus, olivetol synthase was integrated into E. coli adhE locus, idsA was integrated into E. coli idhA locus, fadD into yahK locus.

### Example 6—Identification and Characterization of CBGA Transporters

CBGA transporters were identified and characterized in proprietary E. coli strain L20733. This strain comprises hexanoyl-CoA ligase, prenyltransferase, olivetolic acid cyclase, olivetol synthase, ipk, idi, idsA, and thiM.

Experiment #1: CBGA production was tested by culturing E. coli L20733 in the presence of OLA and prenol at different ODs. Two fermentation tanks inoculated with L20733 were run in batch mode with 2% glycerol and 10 g/L Cas amino acids mixed with a proprietary small scale media (SSMS). To one tank, 0.04 mM OLA and 20 mM prenol at ˜OD10 was added and the same amount of OLA and prenol was added to the second tank at ˜OD30.

Experiment #2: This experiment was similar to Experiment #1 except that different ratios of OLA and prenol were used at higher ODs. Again, two fermentation tanks were used in batch mode. The initial media contained 30 g/L glucose and proprietary fermentation media (FM23). The feed comprised 100 g/L glucose in FM23 media fed at 10 mL/hr. The two tanks received approximately 1 mM OLA and 6-9 mM prenol which was added to the first tank at ˜OD₆₀₀ of 30 and the second tank at ˜OD₆₀₀ of 50.

In both experiments, transcriptomics samples were taken two hours before and two hours after the OLA/prenol addition. Briefly, the samples were taken from the fermentation broth, RNA was isolated and cDNA libraries were prepared and sequenced using an Illumina MiSeq device. The resulting reads were aligned using Bowtie 2 software (John Hopkins University). The counts were calculated using htseq-counts software.

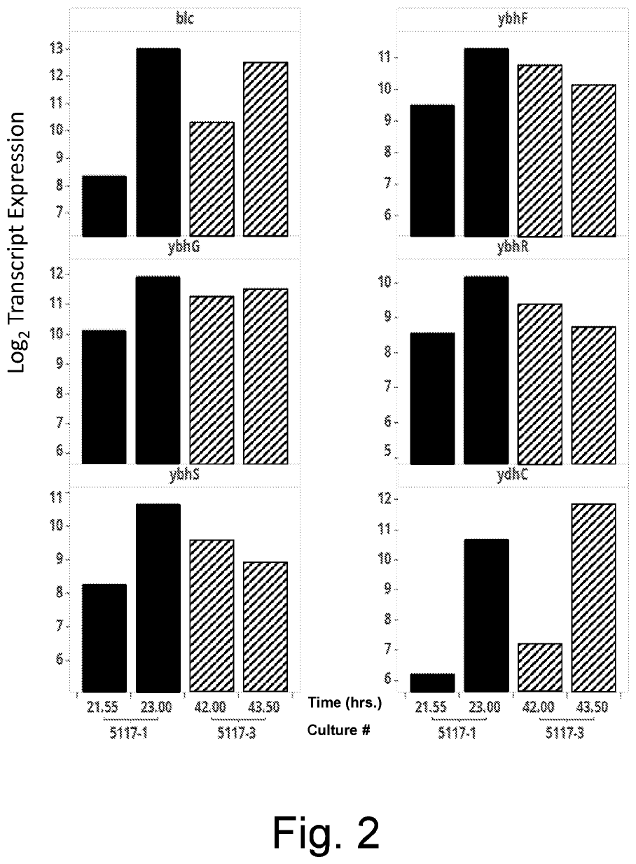

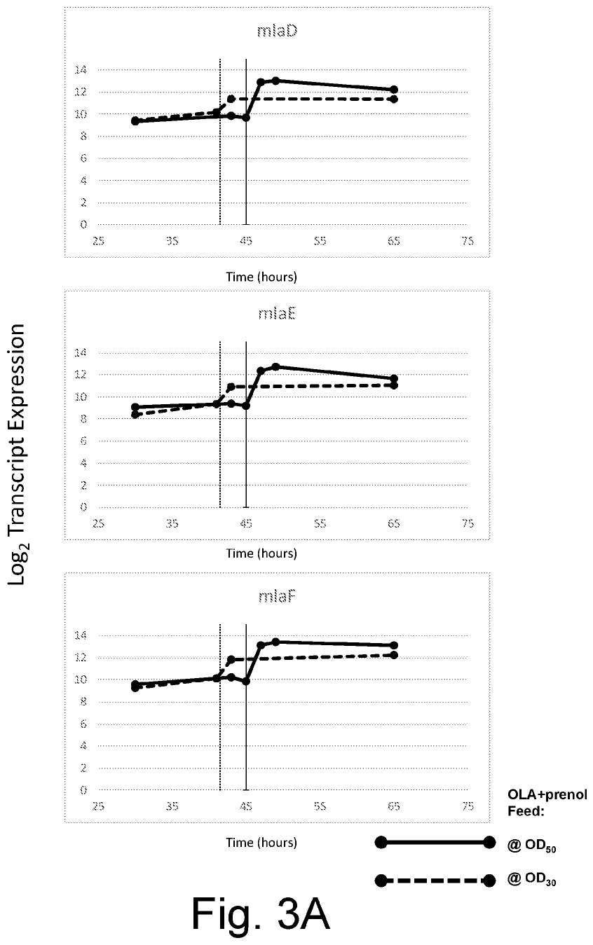

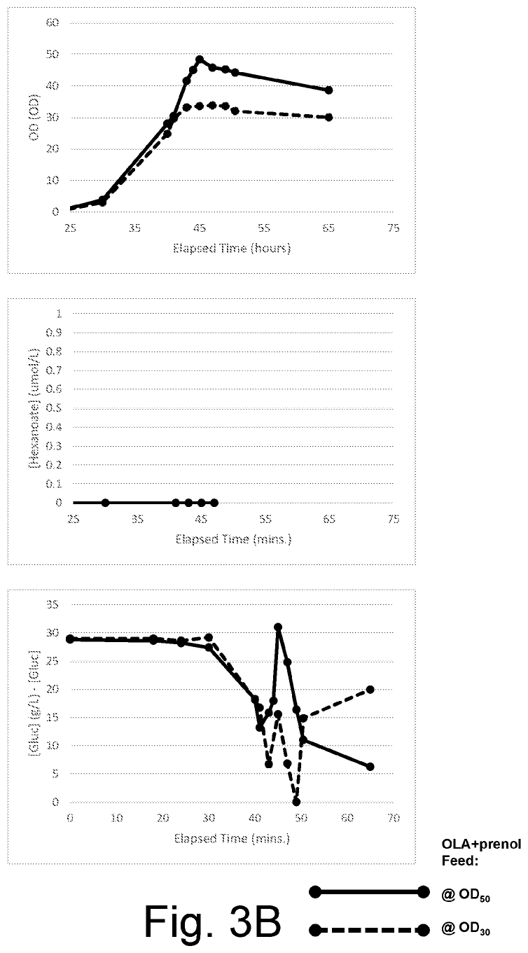

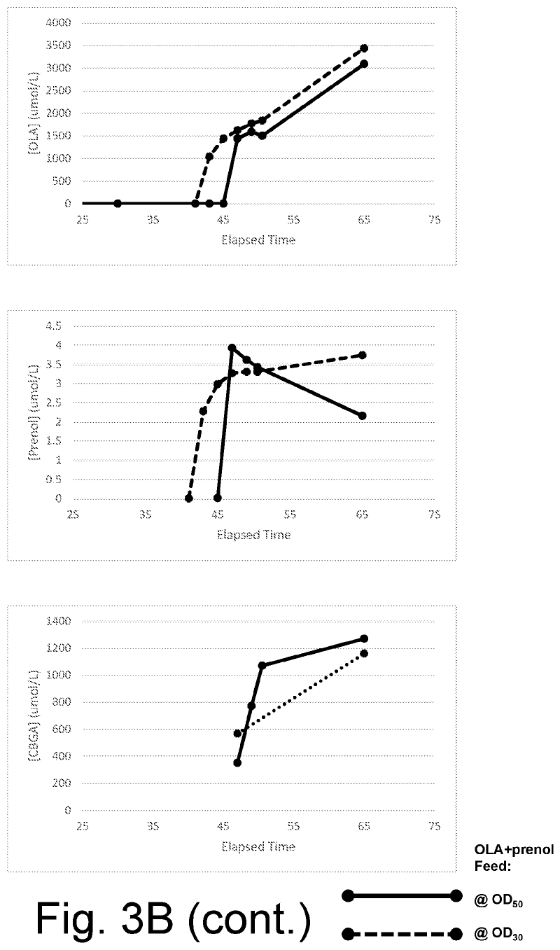

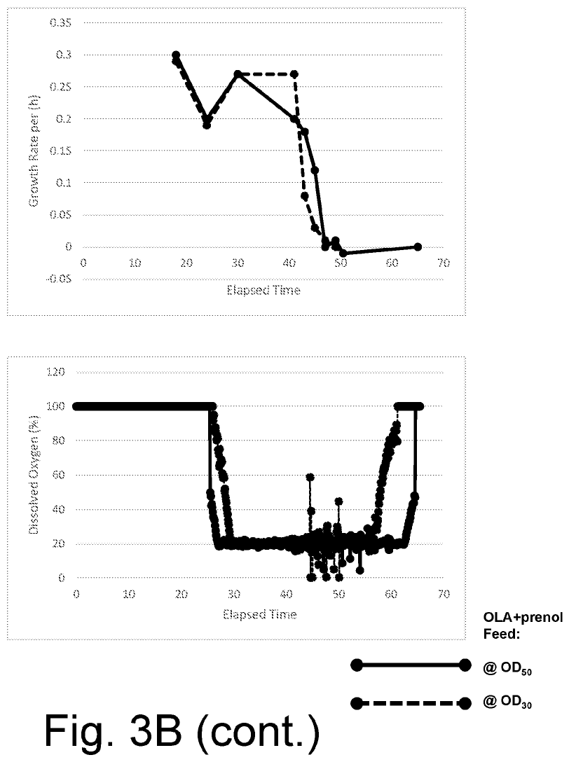

The results of Experiments #1 and #2 are shown in FIGS. 2 and 3. FIG. 2 demonstrates that the OLA/prenol “spike” and subsequent increase in CBGA production caused a significant increase in the expression of blc and certain members of the ybh operon. Culture #5117-1 received an OLA+ prenol spike at 22 hrs, and culture #5117-3 received an OLA+prenol spike at 42.5 hrs (after growing longer). The OLA+prenol spike activates CBGA production, and the results as shown in the graphs compares the expression of the genes before and after the spike. In particular, ybhC expression is strongly increased.

FIG. 3A identified three additional putative transporters whose expression was increased in response to the OLA/prenol spike: mlaD, mlaE, and mlaF. Cultures were grown to either OD₃₀ (dashed line) or OD₅₀ (solid line) and then provided with an OLA+ prenol feed. FIG. 3B illustrates some general parameters of the cultures following the OLA/prenol spike. For validation purposes, rapid spikes in the extracellular concentration of prenol and OLA were observed. The timing of these measured increases in OLA and prenol are indicated in FIG. 3A relative to the increased expression of malD, mlaE, and mlaF. As expected, the OLA/prenol spike did not cause a meaningful change in extracellular concentration of hexanoate despite significantly elevated CBGA production and oxygen utilization, and reduced growth rate.

### Example 7—fadR Deletion Increases OLA Production

FadR is a transcriptional regulator in E. coli fatty acid degradation pathway. It represses the transcription of E. coli β-oxidation genes, e.g., fadE, fadM, fadD, fadI, fadL genes. In addition, FadR upregulates fatty acid biosynthesis genes, e.g., accA, fabA, accB, accC, accD, fabB, fabH, fadD, fabG, fabD. The fadR gene was deleted in E. coli using CRISPR as described in Example 1 to increase the availability of malonyl CoA and thereby increase the production of OLA. The E. coli Strain 1 comprises OLS, OAC, prenyltransferase, thiM, fadD, Mucor circinelloides acc, and deletion of E. coli fadE genes. The E. coli Strain 2 comprises OLS, OAC, prenyltransferase, thiM, fadD, Mucor circinelloides acc, and deletion of E. coli fadE and fabF genes. The guide sequences used for fadR deletion were ATCGGGATGCTGACGAAACG (SEQ ID NO: 206) and CATTAAGGCGCAAAGCCCGG (SEQ ID NO: 207).

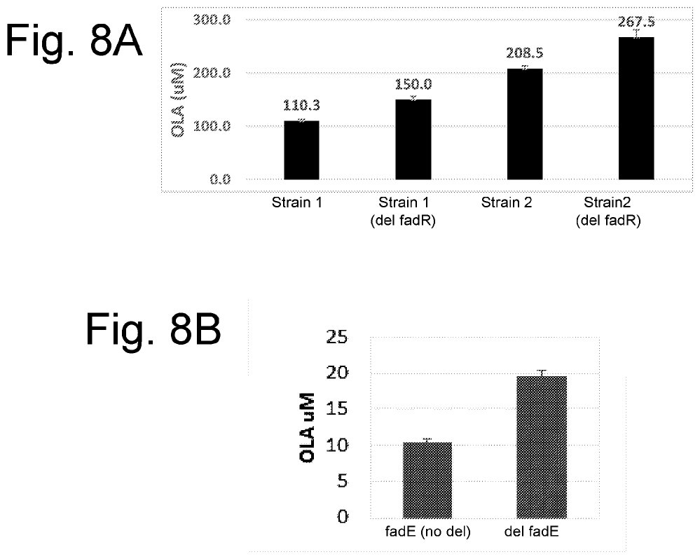

The deletion of fadR gene increases the OLA production (FIG. 8A).

### Example 8—FadE Deletion Increases OLA Production

The fadE gene was deleted in E. coli using CRISPR as described in Example 1. The E. coli strain comprising a pCDF plasmid overexpressing fadD and E. coli accABCD under an IPTG-inducible T7 promoter and a pET plasmid overexpressing OLS and OAC under a cumate-inducible promoter. Deletion of fadE resulted in an increase of OLA production by E. coli (FIG. 8B).

### Example 9—Production of CBGA in a Genetically Modified E. coli Strain

Strain 12482 comprises an integrated geranyl diphosphate synthase from Abies grandis (GenBank accession #: AAN01134.1) and strain 12558 comprises a Geranylgeranyl pyrophosphate synthase from Corynebacterium glutamicum (GenBank accession #: WP_011014931.1). Both strains were engineered to overexpress additional GPP pathway genes thiM, idi, and ipk, OLS, OAC, hexanoyl-CoA ligase. The strains further comprise a prenytransferase on a plasmid.

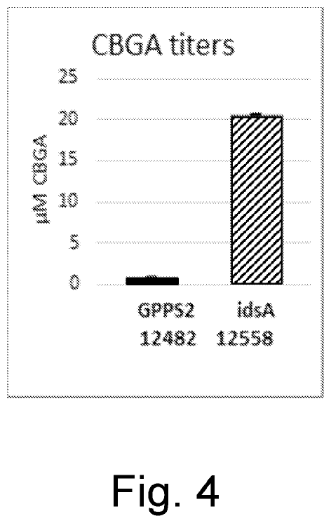

FIG. 4 shows the production of CBGA by the genetically modified E. coli strains.

### Example 10—Production of CBGA in a Genetically Modified E. coli Strain Comprising a Deletion of nudB Gene

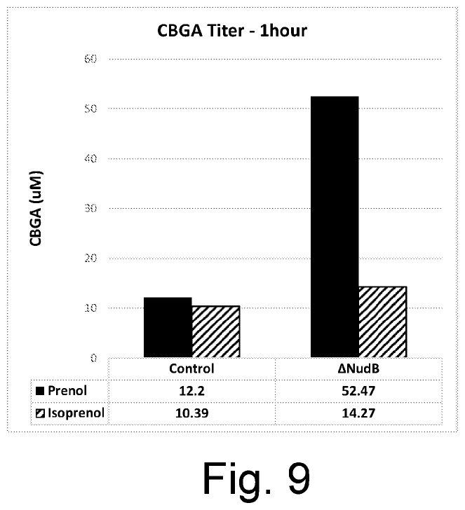

E. coli strains comprising Mucor circinelloides acc, OLS, OAC, GPP pathway genes (thiM, IPK, idi, and idsA), prenyltransferase, deletion of fabF, fadE, fadR genes and were fed with 400 μM OLA and 20 mM prenol or isoprenol, and CBGA production was measured after 1 hr. Deletion of nudB gene increases the production of CBGA (FIG. 9).

The combinations set forth herein are not intended to be limiting. Any of the embodiments of modifications of engineered cells, culture compositions, and methods of producing a cannabinoid (e.g., CBGA) may be combined within the scope of the invention.

### Example 11—Downregulation of fabD Increases the Total OLA Pathway Flux

The in vivo assay procedure was performed as described in Example 1. The analytical method was performed as described in Example 1.

FabD is a malonyl CoA-acyl carrier protein transacylase that is involved in the fatty acid biosynthesis. The first committed step of fatty acid biosynthesis is the conversion of acetyl-CoA to malonyl-CoA by ACC gene(s) followed by the conversion of malonyl-CoA to malonyl-ACP through FabD. The protein expression of fabD was decreased by introducing mutations in its ribosomal binding site (RBS) sequence, and therefore, to increase the availability of malonyl CoA for the OLA pathway. The parental E. coli Strain 13883 comprises Mucor circinelloides ACC, fadD, OLS, OAC, and Wild Type fabD genes. The E. coli fabD strains FabD60, FabD24, FabD41, FabD46, FabD22, FabD12, FabD28, FabD30, FabD5, FabD1, FabD23, FabD13 comprises Mucor circinelloides ACC, fadD, OLS, OAC, and modified fabD genes. The modifications are in the fabD RBS sequence.

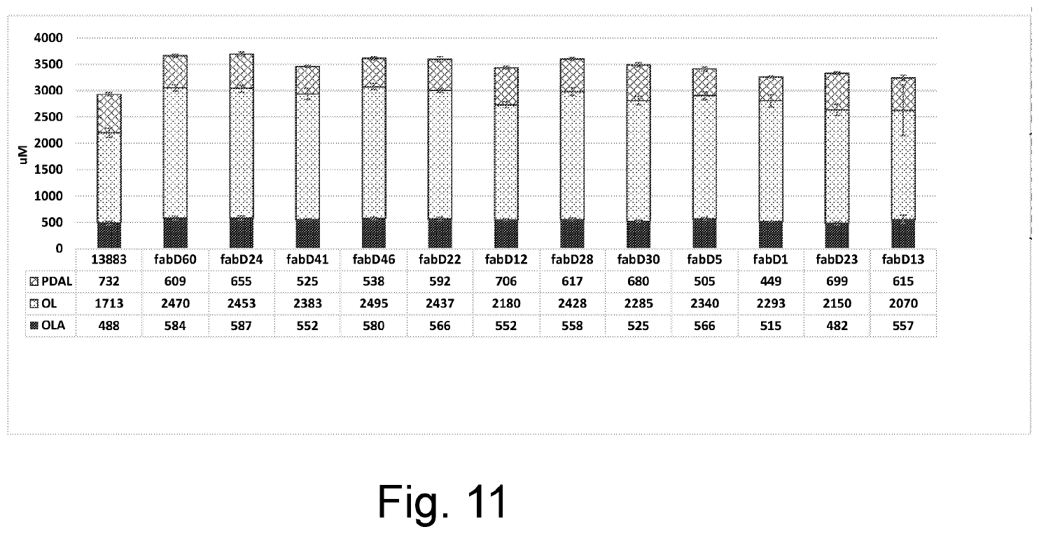

The results are presented in FIG. 11 showing downregulation of fabD on the total OLA pathway flux in E. coli strains comprising ACC, fabD, OLS, and OAC.

### Example 12: Proteomic Analysis of the Effect of Modifications at the fabD RBS on the Down Regulation of FabD Protein Expression

Strains L24075 and L24105 are genetically identical except that in L24105, nudB gene is deleted and the ribosomal binding site (RBS) of FabD is mutated to lower the expression of FabD. Samples were taken from small scale cultures comprising these strains after 24 hours of growth on 4.5 mM hexanoic acid, 24 hr. 0.1 mM biotin, 1 mg/L thiamine and glycerol in the presence or absence of 20 g/L of cas amino acid. Samples were then prepared for tandem mass tag proteomics and analyzed for on the signal of 7 detected peptides unique to fabD across all samples. The strain L24105 having the FabD RBS variation showed a nearly 3-fold drop in FabD protein signal indication the downregulation of FabD expression.

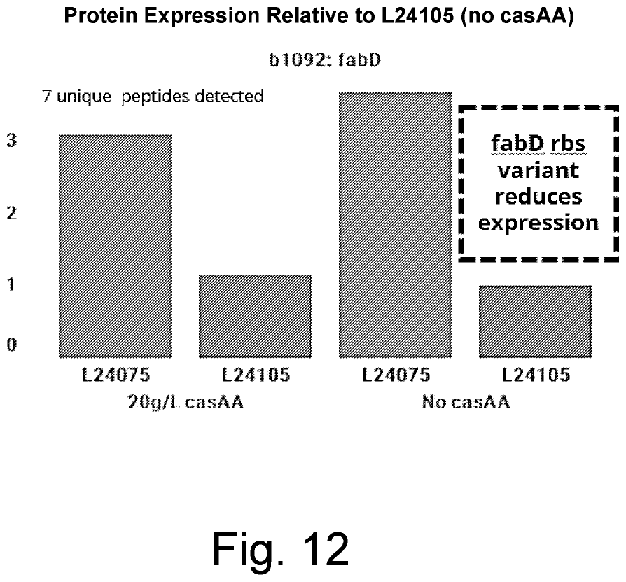

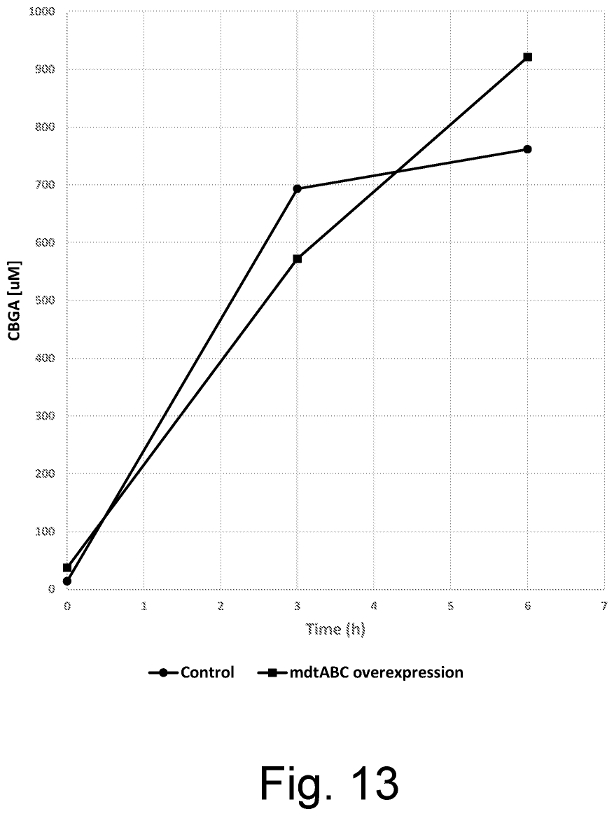

The results are presented in FIG. 12 showing the proteomic analysis of effect of FabD ribosomal binding site (RBS) variation on the expression of FabD.

### Example 13—Overexpression of MdtABC Sustains CBGA Production Rate

MdtABC is one of several multi-drug efflux transporter system from the RND family in E. coli. The three-component transporter (two transmembrane and one periplasmic domain) was overexpressed from a plasmid with a medium-strength constitutive promoter in a CBGA producing E. coli strain.

The in vivo assay procedure was performed as described in Example 1. The analytical method was performed as described in Example 1.

As shown in FIG. 13, in the parental CBGA producing strain CBGA production decreased over time. In contrast, when MdtABC was overexpressed CBGA production was sustained over the duration of the experiment. The results indicate that in the parental CBGA producing strain efflux of CBGA limits CBGA production, and the mdtABC efflux system is capable of exporting CBGA and thereby when overexpressed increases the production of CBGA.

The invention has been described broadly and generically herein. Each of the narrower species and subgeneric groupings falling within the generic disclosure also form part of the methods. This includes the generic description of the methods with a proviso or negative limitation removing any subject matter from the genus, regardless of whether or not the excised material is specifically recited herein.

Other embodiments are within the following claims. In addition, where features or aspects of the methods are described in terms of Markush groups, those skilled in the art will recognize that the invention is also thereby described in terms of any individual member or subgroup of members of the Markush group.

1. An engineered cell for producing a cannabinoid or derivative thereof, wherein the engineered cell comprises
one or more of the following modifications:
(i) express an exogenous nucleic acid sequence encoding an olivetol synthase;
(ii) express an exogenous nucleic acid sequence encoding an olivetolic acid cyclase;
(iii) express an exogenous nucleic acid sequence encoding a prenyltransferase; and
one or more of the following modifications:
(iv) express one or more exogenous nucleic acid sequences or overexpress one or more endogenous genes encoding a protein having an ABC transporter permease activity;
(v) express one or more exogenous nucleic acid sequences or overexpress one or more endogenous genes encoding a protein having an ABC transporter ATP-binding protein;
(vi) express one or more exogenous nucleic acids sequences or overexpress one or more endogenous genes that encodes a protein that is at least 60% identical to: the blc gene product of SEQ ID NO: 147, the ybhG gene product of SEQ ID NO: 116, or the ydhC gene product of SEQ ID NO: 148, or a protein of one of SEQ ID NOs: 210-214;
(vii) express one or more exogenous nucleic acids sequences or overexpress one or more endogenous genes that encodes a protein that is at least 60% identical to the mlaD gene product of SEQ ID NO: 149, the mlaE gene product of SEQ ID NO: 150, or the mlaF gene product of SEQ ID NO: 151;
(viii) express one or more exogenous nucleic acid sequences or overexpress one or more endogenous genes encoding a protein having a siderophore receptor protein activity;
(ix) a disruption of or downregulation in the expression of a regulator of expression of one or more endogenous genes encoding a protein having an ABC transporter permease activity, a protein having an ABC transporter ATP-binding protein activity, a blc gene, a ybhG protein, a ydhC protein, an EmrB/QacA subfamily drug resistance transporter, a mlaD protein, mlaE protein, mlaF protein, or a protein having a siderophore receptor protein activity;
(x) express an exogenous nucleic acid encoding a multi-domain protein having acetyl-CoA carboxylase activity (MD-ACC);
(xi) overexpress one or more endogenous genes encoding acetyl-CoA carboxyltransferase subunit α, biotin carboxyl carrier protein, biotin carboxylase, or acetyl-CoA carboxyltransferase subunit β, or express one or more exogenous genes encoding acetyl-CoA carboxyltransferase, biotin carboxyl carrier protein, or biotin carboxylase;
(xii) disruption of or downregulation in the expression of an endogenous gene encoding a protein having (acyl-carrier-protein)S-malonyltransferase activity, an endogenous gene encoding a protein having 3-hydroxypalmitoyl-(acyl-carrier-protein) dehydratase activity, or both;
(xiii) express an exogenous nucleic acid sequence or overexpress an endogenous gene encoding a protein having fatty acyl-CoA ligase activity, or both;
(xiv) disruption of or downregulation in the expression of at least one endogenous gene encoding a protein having acyl-CoA dehydrogenase activity or enoyl-CoA hydratase activity;
(xv) a disruption or downregulation in the expression of at least one endogenous gene encoding a protein having acyl-CoA esterase/thioesterase activity;
(xvi) disruption of or downregulation in the expression of at least one endogenous gene encoding a repressor of transcription of one or more genes required for fatty acid beta-oxidation or an upregulator of fatty acid biosynthesis in combination with disruption or downregulation of one or more endogenous genes encoding one or more proteins of fatty acid beta-oxidation pathway;
(xvii) express one or more exogenous nucleic acid sequences or overexpress one or more endogenous genes encoding a protein having isopentenyl phosphate kinase activity, isoprenol diphosphokinase activity, prenol kinase activity, prenol diphosphokinase activity, dimethylallyl phosphate kinase activity, or isopentenyl diphosphate isomerase activity;
(xviii) express an exogenous nucleic acid sequence or overexpress an endogenous gene encoding a protein having GPP synthase activity;
(xix) express one or more exogenous nucleic acid sequences or overexpressing one or more endogenous genes encoding one or more enzymes of MVA pathway, MEP pathway, or a non-MVA, non-MEP pathway;
(xx) express an exogenous nucleic acid sequence or overexpress an endogenous gene encoding a biotin-(acetyl-CoA carboxylase) ligase;
(xxi) overexpress an endogenous gene encoding an isopentenyl-diphosphate delta-isomerase or express an exogenous nucleic acid sequence encoding an isopentenyl-diphosphate delta-isomerase;
(xxii) overexpress an endogenous genes encoding a hydroxyethylthiazole kinase or express an exogenous nucleic acid sequence encoding a hydroxyethylthiazole kinase;
(xxiii) express an exogenous nucleic acid sequence encoding a Type III pantothenate kinase or overexpress an endogenous gene encoding a Type III pantothenate kinase;
(xxiv) a disruption of or downregulation in the expression of at least one endogenous gene encoding a phosphatase selected from the group consisting of ADP-sugar pyrophosphatase, dihydroneopterin triphosphate diphosphatase, pyrimidine deoxynucleotide diphosphatase, pyrimidine pyrophosphate phosphatase, and Nudix hydrolase;
(xxv) express one or more exogenous nucleic acid sequences or overexpress one or more endogenous genes encoding a protein having a resistance-nodulation-cell division (FENNY) transporter;
(xxvi) express one or more exogenous nucleic acid sequences or overexpress one or more endogenous genes encoding a protein having a prokaryotic small multidrug (SMR) transporter; and
(xxvii) express one or more exogenous nucleic acid sequences or overexpress one or more endogenous genes encoding a protein that is a member of the major facilitator superfamily (MFS),
wherein the engineered cell produces a cannabinoid or derivative thereof.

2. The engineered cell of claim 1, wherein the MD-ACC has an enzymatic activity of EC 6.4.1.2.

3. The engineered cell of claim 1, wherein the MD-ACC is a fungal MD-ACC, optionally wherein the fungal MD-ACC is derived from Mucor spp, Rhizopus spp. Aspergillus spp, Saccharomyces spp., or Yarrowia sp.

4. (canceled)

5. The engineered cell of claim 1, wherein the MD-ACC has a sequence that is at least 60%, at least 65%, at least 70%, at least 75%, at least 80%, at least 85%, at least 90%, at least 95%, at least 97%, or at least 99% identical to any one of the sequences of SEQ ID NOs: 1-100 and 208-209.

6. The engineered cell of claim 1,
wherein the MD-ACC has a sequence that is at least 60% identical to SEQ ID NO: 1, optionally wherein the MD-ACC protein comprises the sequence of SEQ ID NO: 1; or
wherein the MD-ACC is encoded by a nucleic acid sequence that is at least 60% identical to SEQ ID NO: 101, optionally wherein the MD-ACC is encoded by a nucleic acid sequence comprising SEQ ID NO: 101.

7-9. (canceled)

10. The engineered cell of claim 1, wherein the exogenous nucleic acid encoding a multi-domain protein having acetyl-CoA carboxylase activity (MD-ACC) is heterologous to the cell.

11. The engineered cell of claim 1, wherein the protein having ABC transporter permease activity has an enzymatic activity of EC 7.6.2.2,
optionally wherein the protein having ABC transporter permease activity is at least 60% identical to SEQ ID NO: 113, optionally encoded by ybhS gene;
optionally wherein the protein having ABC transporter permease activity is at least 60% identical to SEQ ID NO: 115, optionally encoded by ybhR gene;
optionally wherein the protein having ABC transporter permease activity is at least 60% identical to SEQ ID NO: 190, optionally encoded by UniProt protein sequence Q8XYF0;
optionally wherein the protein having ABC transporter permease activity is at least 60% identical to SEQ ID NO: 191, optionally encoded by UniProt protein sequence Q8XYE9; or
optionally wherein the protein having multidrug ABC transporter permease activity is at least 60% identical to SEQ ID NO: 114, or optionally encoded by ybhF gene.

12-21. (canceled)

22. The engineered cell of claim 1 wherein the one or more exogenous nucleic acid sequences or the one or more endogenous genes encodes a protein of any one of SEQ ID NOs: 210-214.

23-26. (canceled)

27. The engineered cell of claim 1, wherein the protein having siderophore receptor protein activity is at least 60% identical to SEQ ID NO: 192, optionally having UniProt protein sequence Q8XYF1.

28. (canceled)

29. The engineered cell of claim 1, wherein the cell is engineered for a modification that causes a disruption or downregulation in the expression of an endogenous gene encoding:
(a) a protein having (acyl-carrier-protein)S-malonyltransferase activity, an endogenous gene encoding a protein having 3-hydroxypalmitoyl-(acyl-carrier-protein) dehydratase activity, or both;
(b) a protein having 3-oxoacyl-[acyl-carrier-protein] synthase activity, an endogenous gene encoding a protein having enoyl-[acyl-carrier-protein] reductase activity, or both.
(c) a protein having acyl-CoA dehydrogenase activity or enoyl-CoA hydratase activity;
(d) a protein having acyl-CoA esterase/thioesterase activity; or
(e) a repressor of transcription of one or more genes required for fatty acid beta-oxidation or an upregulator of fatty acid biosynthesis in combination with disruption or downregulation of one or more endogenous genes encoding one or more proteins of fatty acid beta-oxidation pathway.

30-41. (canceled)

42. The engineered cell of claim 1, wherein the cell is engineered to express an exogenous nucleic acid sequence or overexpress an endogenous gene encoding a protein having fatty acyl-CoA ligase activity, optionally wherein the protein having fatty acyl-CoA ligase activity has an enzymatic activity of EC 6.2.1.3, optionally wherein the exogenous nucleic acid sequence or the endogenous gene encodes a protein at least 60% identical to SEQ ID NO: 104, optionally wherein the exogenous nucleic acid sequence or the endogenous gene is a fadD gene or a variant thereof, optionally wherein the engineered cell increases the availability of alkanoyl-CoA as compared to a control cell that is substantially identical to the engineered cell with the exception that the control cell does not comprise one or more of such modifications.

43-65. (canceled)

66. The engineered cell of claim 1, wherein the cell is engineered to:
(a) express an exogenous nucleic acid sequence or overexpress an endogenous gene encoding a protein having isopentenyl phosphate kinase activity, optionally wherein the protein having isopentenyl phosphate kinase activity has an enzymatic activity of EC 2.7.4.26, optionally wherein protein having isopentenyl phosphate kinase activity encodes a protein having at least 60% amino acid sequence identity with SEQ ID NO: 110,
(b) express an exogenous nucleic acid sequence or overexpress an endogenous gene encoding a protein having GPP synthase activity, optionally wherein the protein having geranyl pyrophosphate (GPP) synthase activity has an enzymatic activity of EC 2.5.1, optionally encoding a protein at least 60% identical to SEQ ID NO: 111 or 112, optionally encoded by an IspA gene or an IdsA gene,
(c) express an exogenous nucleic acid sequence or overexpress an endogenous gene encoding a protein having hydroxyethylthiazole kinase activity;
(d) express an exogenous nucleic acid sequence or overexpress an endogenous gene encoding a protein having isopentenyl-diphosphate delta-isomerase activity;
(e) express one or more exogenous nucleic acid sequences or overexpress one or more endogenous genes encoding protein(s) having prenol kinase activity, prenol diphosphokinase activity, isoprenol kinase activity, isoprenol diphosphokinase activity, dimethylallyl phosphate kinase activity, isopentenyl diphosphate kinase activity, or isopentenyl diphosphate isomerase activity; or
(f) a combination of one or more of (a)-(e).

67-73. (canceled)

74. The engineered cell of claim 1, wherein the cell is engineered to express an exogenous nucleic acid sequence or overexpress an endogenous gene encoding a protein having Type III pantothenate kinase activity, optionally wherein the protein having Type III pantothenate kinase activity has an enzymatic activity of EC:2.7.1.33, optionally encoding a protein at least 60% identical to SEQ ID NO: 200, optionally encoded by a coaX gene.

75-78. (canceled)

79. The engineered cell of claim 1, wherein the cell is engineered to express one or more endogenous genes, wherein the native promoter of at least one of the one or more endogenous genes is replaced with a promoter that alters the expression of the genes relative to their expression in a control cell with the native promoter.

80-82. (canceled)

83. The engineered cell of claim 1, wherein the cell is engineered to express an exogenous nucleic acid sequence or overexpress an endogenous gene encoding a protein having biotin ligase activity, optionally wherein the protein having biotin ligase activity has an enzymatic activity of EC:6.3.4.15, optionally encoded by a BirA gene.

84. (canceled)

85. (canceled)

86. The engineered cell of claim 1, wherein the cell is selected from the group consisting of bacteria, fungi, yeast, cyanobacteria, and algae, optionally wherein the cell is a bacterial cell, optionally the bacterial cell being E. coli.

87-89. (canceled)

90. The engineered cell of claim 1, wherein the cell is engineered to express one or more exogenous genes or overexpress one or more endogenous genes, and wherein one or more exogenous or endogenous gene is a non-natural variant of the naturally occurring endogenous or exogenous gene, optionally wherein the non-natural variant of the exogenous or endogenous gene comprise one or more amino acid substitutions, insertions, or deletions as compared to the naturally occurring genes.

91. (canceled)

92. The engineered cell of claim 1, wherein the cannabinoid is cannabigerolic acid (CBGA), tetrahydrocannabivarin (THCV), tetrahydrocannabivarinic acid (THCVA), cannabidivarin (CBDV), cannabidivarinic acid (CBDVA), cannabinol (CBN), cannabinolic acid (CBNA), cannabidiol (CBD), cannabidiolic acid (CBDA), cannabichromene (CBC), cannabichromenic acid (CBCA), cannabigerivarin (CBGV), cannabigerivarinic acid (CBGVA), cannabigerol (CBG), Cannabichromevarin (CBCV), Cannabichromevarinic acid (CBCVA), tetrahydrocannabinol (THC), tetrahydrocannabinolic acid (THCA), analogs, or derivatives thereof, or combinations thereof.

93. (canceled)

94. A method for producing a product having malonyl-CoA as a metabolic intermediate in a microbial production pathway of the product, the method comprising
(a) combining one or more carbon sources, an engineered cell of claim 1, and a microorganism cell culture medium to produce a cell culture; and
(b) incubating the cell culture produced in step (a) under conditions that produce the product,
the method optionally further comprising concentrating the cannabinoid product or derivative thereof from the culture media to produce a cannabinoid concentrate, wherein the cannabinoid or derivative thereof is present in a higher concentration in the cannabinoid concentrate than in the culture media.

95-99. (canceled)

100. A composition comprising a cannabinoid produced or derivative thereof by the method of claim 94, optionally wherein the cannabinoid or derivative thereof is present at a concentration of at least 5% (w/v), at a concentration in the range of 5%-99% (w/v), at a concentration in the range of 5%-90% (w/v), or at a concentration in the range of 5%-20% (w/v).

101-105. (canceled)
